(19) United States
(12) Patent Application Publication
(10) Pub. No.: US 2025/0339523 A1
(43) Pub. Date: Nov. 6, 2025

(54) COMBINATION THERAPY FOR THE TREATMENT OF CANCER

(71) Applicant: MERUS N.V., Utrecht (NL)

(72) Inventors: Gianluca LAUS, Utrecht (NL); Cornelis Jacob Johannes George BOL, Utrecht (NL); Andrew Kim JOE, Utrecht (NL)

(21) Appl. No.: 18/832,429

(22) PCT Filed: Jan. 24, 2023
(86) PCT No.: PCT/NL2023/050029

§ 371 Date: Jul. 23, 2024

(30) Foreign Application Priority Data
Jan. 25, 2022 (NL) 2030691

(51) Int. Cl. A61K 39/395; A61K 39/00; A61P 35/00; A61P 35/04

(52) U.S. Cl. CPC A61K 39/39558; A61P 35/00; A61P 35/04; A61K 2039/507; A61K 2039/545

(57) ABSTRACT

The invention relates to the field of binding molecules. In particular, it relates to the field of therapeutic binding molecules for the treatment of diseases involving aberrant cells, such as cancer cells. In particular, it relates to multispecific antibodies that bind an extracellular part of two or more different membrane associated proteins and thereby modulate a biological activity expressed by a cell, and the use of such antibodies in combination therapies.

### TECHNICAL FIELD

The invention relates to the field of binding molecules. In particular, it relates to the field of therapeutic binding molecules for the treatment of diseases involving aberrant cells, such as cancer cells. In particular, it relates to multispecific antibodies that bind an extracellular part of two or more different membrane associated proteins and thereby modulate a biological activity expressed by a cell, and the use of such antibodies in combination therapies.

### BACKGROUND

Cancer is still a major cause of morbidity and death in the world, in spite of the many advances that have been made in the treatment of the disease and the increased knowledge of the molecular events that lead to cancer.

Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases, the tumors in the patients stop growing or temporarily shrink (referred to as remission) only to start proliferating again, sometimes more rapidly (referred to as relapse), and become increasingly more difficult to treat. More recently the focus of cancer drug development has moved away from broadly cytotoxic chemotherapy to targeted cytostatic therapies with less toxicity. Treatment of advanced cancer has been validated clinically in leukemia and some other cancers. However, in a majority of carcinomas, targeted approaches are still proving not to be effective enough to completely abolish cancer in the majority of the patients.

Targeting of cancers has been achieved using a variety of different methods including for instance small molecules directed towards signaling proteins on which the cancer depends for survival and/or growth; vaccines with tumor specific proteins; cell therapies with immune cells that actively kill tumor cells, and antibodies that target cytotoxic molecules to the tumor; interfere with signaling and/or that (re)direct the immune system of the host to the tumor cells.

The (re)direction of the immune system can be accomplished in several ways. One way is by activating T-cell costimulatory molecules such as the tumor-necrosis factor receptor superfamily, including CD137 (4-1BB, TNFRSF9). Activation of CD137 leads to increased T-cell proliferation, cytokine production and prolonged CD8⁺ T-cell survival. Another way is by blocking the negative signals induced by molecules involved in the immune checkpoint, such as cytotoxic T-lymphocyte-associated antigen 4 (CTLA-4), programmed cell-death (PD-1) expressed on T-cells or its cognate ligand programmed cell death 1 ligand 1 (PD-L1) which can be expressed on tumor cells. Upon binding of PD-L1 to PD-1, signal transduction results in the attenuation of T-cell receptor (TCR) signaling and T-cell exhaustion. This is a mechanism used by tumors in order to evade and/or suppress the immune system.

This immune suppression can be blocked by immune checkpoint inhibitor therapies (ICIs) such as antagonistic antibodies against PD-1 or PD-L1. ICI treatment has demonstrated remarkably durable responses in a subset of cancer patients that are correlated with activated CD8⁺ T cell infiltration and proliferation. Combinations of ICIs (e.g., anti-PD-1 and anti-CTLA-4) have been shown to further enhance efficacy, however at the cost of toxicity, as the majority of patients experience grade 3 or 4 treatment-related adverse events.

Dual targeting of the PD-1/PD-L1 axis and CD137 may be beneficial in optimally engaging specific anti-tumor immunity. Currently, the two most advanced therapeutic CD137 agonist antibodies in clinical testing are urelumab (IgG4) and utomilumab (IgG2). Development of urelumab has been halted as a consequence of dose-dependent hepatitis resulting from systemic activation of the CD137 pathway in patients. Safe administration of urelumab required a reduced dose; 0.1 mg/kg was chosen for combination studies with PD-1 inhibitors. Utomilumab is better tolerated by patients but has modest anti-tumor activity as a monotherapy and no clear synergy with PD-1 blockade in combination therapy.

There thus remains a need to provide more and better options for health care professionals to treat cancer, in particular advanced or metastatic solid tumors.

### SUMMARY

The present disclosure relates to a combination therapy, wherein a multispecific antibody that comprises an antigen binding site that binds an extracellular part of CD137 and an antigen binding site that binds an extracellular part of a second membrane protein is used with a PD-L1 or PD-1 inhibitor in a method of treatment of cancer in a subject in need thereof.

The present disclosure provides means and methods for (re)directing immune system components in the treatment of cancer, in particular advanced or metastatic solid tumors.

In certain aspects, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the treatment further comprises administering of a PD-L1 or PD-1 inhibitor.

In certain aspects, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is for simultaneous, sequential or separate administration with a PD-1 or PD-L1 inhibitor.

In certain aspects, the present disclosure provides a combination of a multispecific antibody that comprises an antigen binding site that binds an extracellular part of CD137 and an antigen binding site that binds an extracellular part of a second membrane protein and a PD-L1 or PD-1 inhibitor for use in a method of treatment of cancer in a subject in need thereof.

In certain aspects, the present disclosure provides a method of treating cancer in a subject in need thereof, the method comprising administering a multispecific antibody that comprises an antigen binding site that binds an extracellular part of CD137 and an antigen binding site that binds an extracellular part of a second membrane protein and a PD-1 or PD-L1 inhibitor to the subject in need thereof.

In certain aspects, the multispecific antibody is administered simultaneously, sequentially, or separately with the PD-1 or PD-L1 inhibitor.

In certain aspects, the cancer is an advanced or metastatic solid tumor, such as selected from locally advanced or metastatic lung cancer and locally advanced or metastatic melanoma, such as the cancer is NSCLC. In certain aspects, the melanoma is selected from cutaneous, acral or mucosal melanoma. In certain aspects, the cancer is a Merkel cell carcinoma (also called neuroendocrine carcinoma of the skin or trabecular cancer).

In certain aspects, the cancer is relapsed to a PD-1/PD-L1 therapy and/or is positive for PD-L1 expression.

In certain aspects, the multispecific antibody is administered prior to the PD-1 or PD-L1 inhibitor.

In certain aspects, the multispecific antibody is a bispecific antibody.

In certain aspects, the antigen binding site of the multispecific antibody that binds an extracellular part of a second membrane protein binds PD-L1.

In certain aspects, the present disclosure provides a multispecific antibody for use in a method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof,
wherein the multispecific antibody is for simultaneous, sequential, or separate administration with a PD-1 or PD-L1 inhibitor.

In certain aspects, the present disclosure provides a method of treating cancer in a subject in need thereof, the method comprising administering a multispecific antibody that comprises a binding domain that binds CD137, comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof;
and a PD-1 or PD-L1 inhibitor, to a subject in need thereof.

In certain aspects, the present disclosure provides a multispecific antibody for use in a method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof,
wherein the multispecific antibody is for simultaneous, subsequent or separate administration with a PD-1 or PD-L1 inhibitor;
and wherein the cancer is an advanced or metastatic solid tumor.

In certain aspects, the present disclosure provides a pharmaceutical combination or a kit of parts comprising a multispecific antibody that comprises an antigen binding site that binds an extracellular part of CD137 and an antigen binding site that binds an extracellular part of a second membrane protein and instructions for use of the multispecific antibody in combination with a PD-1 or PD-L1 inhibitor, particularly for the treatment of cancer.

In certain aspects, the present disclosure provides a combination of a multispecific antibody that comprises an antigen binding site that binds an extracellular part of CD137 and an antigen binding site that binds an extracellular part of PD-L1 and a PD-1 or PD-L1 inhibitor for use in the treatment of cancer in a subject in need thereof.

In certain aspects, the present disclosure provides a PD-1 or PD-L1 inhibitor for the treatment of cancer in a subject in need thereof, wherein the PD-L1 inhibitor is for simultaneous or sequential administration with a multispecific antibody that comprises an antigen binding site that binds an extracellular part of CD137 and an antigen binding site that binds an extracellular part of a second membrane protein.

In certain aspects, the present disclosure provides a multispecific antibody that comprises an antigen binding site that binds an extracellular part of CD137 and an antigen binding site that binds an extracellular part of PD-L1 for use in the treatment of cancer in a subject to which a PD-1 or PD-L1 inhibitor has been or will be administered.

In certain aspects, the present disclosure provides a PD-1 or PD-L1 inhibitor for use in the treatment of cancer in a patient to which a multispecific antibody that comprises an antigen binding site that binds an extracellular part of CD137 and an antigen binding site that binds an extracellular part of PD-L1 has been or will be administered.

In certain aspects, the multispecific antibody is administered in a dose of between 25-300 mg, such as between 25-150 or mg 25-100 mg, or such as between 25-50 mg or 50-100 mg.

In certain aspects, the PD-1 inhibitor is Pembrolizumab and is administered in a dose of between 200-600 mg, such as between 300-500, or such as around 400 mg.

In certain aspects, the cancer is an advanced or metastatic solid tumor.

In certain aspects, the multispecific antibody, the PD-L1 or PD-1 inhibitor are isolated antibodies.

In certain aspects, the subject is a human subject.

In certain aspects, the subject has not yet received prior treatment with a CD137 agonist or prior therapy with CAR T cell therapy, or prior treatment including targeted small molecule therapy or radiation therapy.

### DETAILED DESCRIPTION

In certain aspects, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is for simultaneous, sequential, or separate administration with a PD-1 or PD-L1 inhibitor. In certain aspects, the present disclosure provides a method of treating cancer in a subject in need thereof, the method comprising administering a multispecific antibody that comprises an antigen binding site that binds an extracellular part of CD137 and an antigen binding site that binds an extracellular part of a second membrane protein and a PD-1 or PD-L1 inhibitor to the subject.

In certain aspects, the second membrane protein is PD-L1, and the antigen binding site that binds an extracellular part of a second membrane protein, binds PD-L1.

CD137 can be expressed by activated T-cells. It is also found on other cells such as dendritic cells, natural killer cells, granulocytes and cells of the blood vessel wall at sites of inflammation. The protein is known for its costimulatory activity for activation of T-cells. CD137 is known under a number of different names such as: TNFRSF9; TNF Receptor Superfamily Member 9; Tumor Necrosis Factor Receptor Superfamily Member 9; T-Cell Antigen 4-1BB Homolog; 4-1BB Ligand Receptor; T-Cell Antigen ILA; CD137 Antigen; CDw137; ILA; Interleukin-Activated Receptor, Homolog Of Mouse Ly63; Induced By Lymphocyte Activation (ILA); Homolog Of Mouse 4-1BB; Receptor Protein 4-1BB; T Cell Antigen ILA; and 4-1BB. External Ids for CD137 are HGNC: 11924; Entrez Gene: 3604; Ensembl: ENSG00000049249; OMIM: 602250; and UniProtKB: Q07011. CD137 is an inducible receptor most commonly upregulated on activated CD8+ T cells. CD137 signaling enhances T cell function by activating NF-κB [Arch et al, 1998]. Other cell immune cell types including CD4+ T cells, monocytes, B cells, dendritic cell (DC) subpopulations and granulocytes and NK cells can express CD137 at various levels [Shao et al, 2011]. In monocytes, CD137 is inducible by activation with lipopolysaccharide (LPS) and IL-1b. In B lymphocytes, CD137 expression is induced by antibodies against cell-surface immunoglobulin and by transformation with EBV. In DCs, CD137 ligation induces their maturation through upregulation of B7 co-stimulatory molecules (CD80 and CD86), in addition to enhancing their production of inflammatory cytokines (IL-6 and IL-12) and their survival [Makkouk et al, 2015]. The natural function of CD137 ligation on neutrophils is the increment of phagocytosis of bacterial and parasitic infections. In addition ligation of CD137 blocks the anti-apoptosis signals mediated by the IL-3/IL-5/GM-CSF receptors in neutrophils and eosinophils in vitro, thereby preventing granulocyte accumulation [Simon, 2001; Vinay et al, 2011]. In non-lymphoid cells such as chondrocytes, endothelial cells and tumor cells CD137 expression is driven by cytokine stimulation such as IL-1b for chondrocytes, the inflammatory cytokines TNFalpha/IFNγ/IL-1b for endothelial cells and IFNγ for tumor cells. The ligand that stimulates CD137 (CD137L) is expressed on activated antigen presenting cells. CD137 exists in the membrane as monomers and dimers [Pollok et al, 1993].

The B7 family comprises a number of structurally related, cell-surface proteins, which bind to receptors on lymphocytes that regulate immune responses. Activation of lymphocytes is initiated by engagement of cell-surface, antigen-specific T-cell receptors or B-cell receptors. Additional signals delivered simultaneously by B7 ligands further determine the immune response of these cells. These so-called ‘costimulatory’ or ‘coinhibitory’ signals are delivered by B7 family members through the CD28 family of receptors on lymphocytes. Binding of B7-family members with costimulatory receptors augments immune responses, and binding with coinhibitory receptors attenuates immune responses. Presently the following members are believed to be part of this family: B7.1 (CD80), B7.2 (CD86), inducible costimulator ligand (ICOS-L), programmed death-1 ligand (PD-L1), programmed death-2 ligand (PD-L2), B7-H3 (CD276), B7-H4, B7-H5, B7-H6 and B7-H7. B7 family members are expressed in lymphoid and non-lymphoid tissues. Effects of members on regulating immune responses are shown in the development of immunodeficiency and autoimmune diseases in mice with mutations in B7-family genes. Manipulation of the signals delivered by B7 ligands has shown potential in the treatment of autoimmunity, inflammatory diseases and cancer.

PD-L1 is a type 1 transmembrane protein that plays a role in suppressing an immune response during particular events such as pregnancy, tissue allografts, autoimmune disease and other disease states such as hepatitis. PD-L1 is expressed in various types of cancers, especially in NSCLC (Boland et al., 2013; Velcheti et al., 2014), melanoma, renal cell carcinoma, gastric cancer, hepatocellular as well as various leukemias and multiple myeloma (Bernstein et al., 2014; Thompson et al., 2005). PD-L1 is present in the cytoplasm and plasma membrane of cancer cells, but not all cancers or all cells within a tumor express PD-L1 (Dong et al., 2002). Multiple tumor microenvironment cells contribute to immune suppression by upregulating PD-L1 expression. This effect is called “adaptive immune resistance”, because the tumor protects itself by inducing PD-L1 in response to IFN-γ produced by activated T cells (Sharma et al., 2017). PD-L1 can also be regulated by oncogenes, this mechanism is known as inherent immune resistance (Akbay et al., 2013). Within the tumor microenvironment, PD-L1 is also expressed on myeloid cells and activated T cells (Tumeh et al., 2014). The expression of PD-L1 is induced by multiple proinflammatory molecules, including types I and II IFN-γ, TNF-α, LPS, GM-CSF and VEGF, as well as the cytokines IL-10 and IL-4, with IFN-γ being the most potent inducer (Sznol and Chen, 2013).

Programmed Cell Death 1 protein (PD-1) is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in down regulating the immune system by inhibiting the activation of T-cells, which in turn reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. New classes of drugs that block the activity of PD-1, the PD-1 inhibitors, activate the immune system to attack tumors and are therefore used with success to treat some types of cancer.

The binding of PD-L1 to PD-1 or B7.1 (CD80) transmits an inhibitory signal which reduces the proliferation of the PD-1 expressing T cells. PD-1 is thought to be able to control the accumulation of foreign antigen specific T cells through apoptosis. PD-L1 is expressed by a variety of cancer cells and the expression thereof is thought to be at least in part responsible for a dampening of an immune response against the cancer cell. PD-L1 is a member of the B7-family of protein and is known under a variety of other names such as CD274 Molecule; CD274 Antigen; B7 Homolog 1; PDCD1 Ligand 1; PDCD1LG1; PDCD1L1; B7H1; PDL1; Programmed Cell Death 1 Ligand 1; Programmed Death Ligand 1; B7-H1; and B7-H. External Ids for CD274 are HGNC: 17635; Entrez Gene: 29126; Ensembl: ENSG00000120217; OMIM: 605402; UniProtKB: Q9NZQ7.

PD-L2 is a second ligand for PD-1. Engagement of PD-1 by PD-L2 inhibits T cell receptor (TCR)-mediated proliferation and cytokine production by CD4+ T cells. At low antigen concentrations, PD-L2/PD-1 binding inhibits B7-CD28 signals. At high antigen concentrations, PD-L2/PD-1 binding reduces cytokine production. PD-L expression is up-regulated on antigen-presenting cells by interferon gamma treatment. It is expressed in some normal tissues and a variety of tumors. PD-L1 and PD-L2 are thought to have overlapping functions and regulate T cell responses. The protein is known under a number of other names such as Programmed Cell Death 1 Ligand 2; B7 Dendritic Cell Molecule; Programmed Death Ligand 2; Butyrophilin B7-DC; PDCD1 Ligand 2; PD-1 Ligand 2; PDCD1L2; B7-DC; CD273; B7DC; PDL2; PD-1-Ligand 2; CD273 Antigen; BA574F11.2; and Btdc. External Ids for PD-L2 are HGNC: 18731; Entrez Gene: 80380; Ensembl: ENSG00000197646; OMIM: 605723; and UniProtKB: Q9BQ51.

PD-1 inhibitors are known in the art. They include antibodies that bind and block PD-1. Pembrolizumab is a humanized antibody used in cancer immunotherapy that treats melanoma, lung cancer, head and neck cancer, Hodgkin's lymphoma, stomach cancer, and cervical cancer. It is given by slow injection into a vein. Pembrolizumab is a therapeutic antibody that binds to and blocks PD-1 located on lymphocytes. This receptor is a so-called “immune checkpoint”, and thus is generally responsible for preventing the immune system from attacking the body's own tissues. Normally, the PD-1 receptor on activated T-cells binds to the PD-L1 or PD-L2 ligands present on normal cells in the body, deactivating any potential cell-mediated immune response against these cells. Many cancers make proteins such as PD-L1 that also bind to the PD-1 receptor, thus shutting down the ability of the body to kill the cancer. Pembrolizumab works by inhibiting lymphocytes PD-1 receptors, blocking the ligands that would deactivate it and prevent an immune response. This allows the immune system to target and destroy cancer cells, but also blocks a key mechanism preventing the immune system from attacking the body itself.

Tumors often have mutations that cause impaired DNA mismatch repair. This in turn often results in microsatellite instability allowing the tumor to generate numerous mutant proteins that could serve as tumor antigens, triggering an immune response against the tumor. By preventing the self-checkpoint system from blocking the T-cells, [14][30] pembrolizumab appears to facilitate clearance of any such tumor by the immune system.

Pembrolizumab was approved for medical use in the United States in 2014. In 2017, the US Food and Drug Administration (FDA) approved it for any unresectable or metastatic solid tumor with certain genetic anomalies (mismatch repair deficiency or microsatellite instability).

In certain aspects, the multispecific antibody according to the use or method of the present disclosure binds to a second membrane protein that is not a member of the TNF receptor superfamily. In certain aspects, the second membrane protein is a member of the B7 family. In certain aspects, the second membrane protein is PD-L1 or PD-L2, in certain aspects PD-L1.

In certain aspects, the multispecific antibody according to the use or method of the present disclosure comprises one antigen binding site that binds to the PD-1 binding domain of PD-L1.

In certain aspects, the multispecific antibody according to the use or method of the present disclosure binds a second membrane protein that is not to a significant extent expressed by a T-cell.

In certain aspects, the multispecific antibody according to the use or method of the present disclosure binds a second membrane protein that is expressed on an antigen presenting cell, a tumor cell, a virus infected cell or a parasite infected cell.

In certain aspects, the multispecific antibody according to the use or method of the present disclosure binds a second membrane protein that is a membrane protein that is present in one or more zones on the cell membrane. In certain aspects, the zone is a cluster, domain, micro-domain or compartment on the cell membrane, in certain aspects an immunological synapse.

In certain aspects, the multispecific antibody according to the use or method of the present disclosure binds a second membrane protein that is present on the cell membrane as a part of a multimeric membrane protein comprising two or more of said second membrane proteins. In certain aspects, the second membrane protein is present on the cell membrane as a part of a homodimer or a homotrimer.

In certain aspects, the multispecific antibody according to the use or method of the present disclosure comprises one antigen binding site that binds to the CD137L binding domain of CD137.

In certain aspects, the multispecific antibody according to the use or method of the present disclosure comprises one antigen binding site that blocks the binding of a ligand to CD137 or binds to an extracellular ligand-blocking binding site of CD137, in certain aspects a CD137L blocking binding site.

In certain aspects, the multispecific antibody according to the use or method of the present disclosure comprises a variable domain that binds an extracellular part of CD137 which is defined as a variable domain that, when in a bivalent monospecific antibody format that comprises two of said variable domains that bind CD137, does not stimulate activity of CD137 on a cell or does so at a reduced level in comparison to one of said variable domain as part of a multispecific antibody having a second variable domain binding a tumor associated antigen, such as a member of the B7 family, or such as PD-L1.

In certain aspects, the variable domain that binds an extracellular part of CD137 is defined as a variable domain that, when in a bivalent monospecific antibody format (such as IgG, in particular IgG1, or human IgG1) that comprises two of said variable domains that bind CD137, does not stimulate activity of CD137 on a cell or does so at a reduced level in comparison to one of said variable domain as part of a bispecific antibody (such as IgG, in particular IgG1 or human IgG1) having a second variable domain binding a tumor-associated antigen. As a skilled person will recognize that when comparing CD137 stimulating activity between the bivalent monospecific antibody and the bispecific antibody referred to above, the antibodies are in the same format (such as IgG, in particular IgG1 or human IgG1), wherein the point of difference between the antibodies being compared is on the one hand a monospecific antibody comprising the presence of the identical variable domain binding CD137 in a bivalent form and on the other hand a bispecific antibody comprising one of said CD137 variable domains and a variable domain binding a tumor-associated antigen, such as variable domains that comprise the amino acid sequence of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5359 (SEQ ID NO: 69); MF5377 (SEQ ID NO: 73); MF5382 (SEQ ID NO: 77); MF5424 (SEQ ID NO: 81); MF5426 (SEQ ID NO: 85); MF5439 (SEQ ID NO: 89); MF5442 (SEQ ID NO: 92); MF5553 (SEQ ID NO: 96); MF5557 (SEQ ID NO: 97); MF5561 (SEQ ID NO: 100); MF5576 (SEQ ID NO: 103); MF5594 (SEQ ID NO: 104); or MF5708 (SEQ ID NO: 107). In certain aspects, said variable domain that binds an extracellular part of CD137 which is non-stimulatory when in bivalent monospecific antibody format comprises the CDR sequences of VHs such as MF6754, MF6808, MF6763, MF6785 or MF6797.

In certain aspects, the multispecific antibody according to the use or method of the present disclosure comprises a variable domain that binds an extracellular part of CD137 which is capable of stimulating activity of CD137 on a cell when combined in a multispecific antibody with a second variable domain which binds to PD-L1, when the multispecific antibody is in the presence of a first cell expressing CD137 and a second cell expressing PD-L1.

In certain aspects, the multispecific antibody according to the use or method of the present disclosure is capable of binding CD137 and PD-L1 simultaneously.

A multispecific antibody according to the present disclosure that binds CD137 and an extracellular part of a second membrane protein, in particular a membrane protein that is a member of the B7 family, provides the advantage that a desired immune response can be particularly well promoted, since B7 family members deliver ‘costimulatory’ or ‘coinhibitory’ signals to lymphocytes, thereby augmenting or attenuating an immune response. Hence, by targeting a second transmembrane protein, in particular a transmembrane protein that is a member of the B7 family, it is possible to enhance stimulatory signals and/or to counteract inhibitory signals, thereby inducing or enhancing a desired immune response, for instance against aberrant cells such as cancer cells. Consequently, according to the present disclosure, the multispecific antibody is used in the treatment of cancer in a subject in need thereof when a desired immune response is elicited against aberrant cells that are found in cancer.

In certain aspects, a multispecific antibody according to the present disclosure has one antigen binding site that can bind an extracellular part of CD137 and a second antigen binding site that can bind an extracellular part of a second membrane protein that is not a member of the TNF receptor superfamily but in certain aspects a member of the B7 family, or such as PD-L1.

This provides the advantage that in cis activation of (immune) cells such as T cells expressing several different members of the TNF receptor superfamily is at least in part avoided, thereby reducing the potential adverse side effects and toxicity due to nonspecific T cell activation. Prior art approaches may lead to T cell activation in cis, meaning in the absence of a second target, and may involve the risk of an excessive T cell response, for instance resulting in a cytokine storm.

Consequently, such prior art approaches have an increased potential of adverse side effects compared to a binding molecule according to the disclosure having an antigen binding site that can bind CD137 and an antigen binding site that can bind an extracellular part of a second membrane protein.

In certain aspects, the present disclosure relates to a multispecific antibody targeting both PD-L1 and CD137 to partly avoid in cis activation of (immune) cells such as T cells. In certain aspects, the variable domain that binds an extracellular part of CD137 is a domain that, when in a bivalent monospecific antibody format that comprises two of such CD137 binding domains, does not stimulate activity of CD137 on a cell or does so at a reduced level in comparison to one of said variable domain as part of a multispecific antibody having a second variable domain binding a tumor associated antigen, in certain aspects a member of the B7 family, or such as PD-L1. Suitable CD137 binding arms are disclosed in WO 2018/056821.

In certain aspects, a multispecific antibody according to the use or method of the present disclosure, may be an agonistic CD137 antibody, for example an antibody that is capable of stimulating activity of CD137. In certain aspects, a multispecific antibody according to the use or method of the present disclosure may be an antagonistic CD137 antibody, for example an antibody that is capable of reducing activity of CD137.

In certain aspects, a multispecific antibody according to the use or method of the present disclosure may be an agonistic B7 antibody, for example an antibody that is capable of stimulating activity of a B7 family member. In certain aspects, a multispecific antibody according to the use or method of the present disclosure may be an antagonistic B7 antibody, for example an antibody that is capable of reducing activity of a B7 family member.

In certain aspects, a multispecific antibody according to the use or method of the present disclosure may be an agonistic PD-L1 antibody, for example an antibody that is capable of stimulating activity of PD-L1. In certain aspects, a multispecific antibody according to the use or method of the present disclosure may be an antagonistic PD-L1 antibody, for example an antibody that is capable of reducing activity of PD-L1.

In certain aspects, a multispecific antibody according to the use or method of the present disclosure may stimulate CD137 activity when bound to a B7 family member. In certain aspects, the multispecific antibody stimulates CD137 activity when bound to CD137 and PD-L1. In certain aspects, the multispecific antibody only induces or activates CD137 signaling in the presence of PD-L1 expressing cells.

In certain aspects, the multispecific antibody according to the use or method of the present disclosure comprises antigen binding sites consisting of one immunoglobulin variable domain that binds to CD137 and one immunoglobulin variable domain that binds to an extracellular part of a second membrane protein.

In certain aspects, variable domains comprised by the multispecific antibody according to the use or method of the present disclosure that bind to an extracellular part of CD137 and that at least partially block the binding of CD137 ligand to CD137 are variable domains that comprise the amino acid sequence of the VH of: MF6783 (SEQ ID NO: 1); MF6861 (SEQ ID NO: 5); MF6795 (SEQ ID NO: 9); MF6808 (SEQ ID NO: 13); MF6798 (SEQ ID NO: 17); MF6754 (SEQ ID NO: 20); MF6763 (SEQ ID NO: 24); MF6744 (SEQ ID NO: 28); MF6785 (SEQ ID NO: 31); MF6825 (SEQ ID NO: 35); MF6737 (SEQ ID NO: 39); MF6749 (SEQ ID NO: 43); MF6788 (SEQ ID NO: 46); or MF6797 (SEQ ID NO: 49).

In certain aspects, variable domains comprised by the multispecific antibody according to the use or method of the present disclosure that bind to an extracellular part of CD137 and that at least partially block the binding of CD137 ligand to CD137 are variable domains that comprise the CDR1, CDR2 and CDR3 sequences of the amino acid sequence of the VH of: MF6783 (SEQ ID NO: 1); MF6861 (SEQ ID NO: 5); MF6795 (SEQ ID NO: 9); MF6808 (SEQ ID NO: 13); MF6798 (SEQ ID NO: 17); MF6754 (SEQ ID NO: 20); MF6763 (SEQ ID NO: 24); MF6744 (SEQ ID NO: 28); MF6785 (SEQ ID NO: 31); MF6825 (SEQ ID NO: 35); MF6737 (SEQ ID NO: 39); MF6749 (SEQ ID NO: 43); MF6788 (SEQ ID NO: 46); or MF6797 (SEQ ID NO: 49).

In certain aspects, a multispecific antibody or a functional part, derivative and/or analogue thereof according to the use or method of the present disclosure that comprises a variable domain that binds to an extracellular part of CD137 comprises a heavy chain variable region with a CDR3 region that comprises the amino acid sequence of the CDR3 region of the variable heavy chain region of MF6808 (SEQ ID NO: 13); MF6754 (SEQ ID NO: 23); MF6763 (SEQ ID NO: 27); MF6785 (SEQ ID NO: 34); or MF6797 (SEQ ID NO: 52).

In certain aspects, a multispecific antibody or a functional part, derivative and/or analogue thereof according to the use or method of the present disclosure that comprises a variable domain that binds to an extracellular part of CD137 comprises a heavy chain variable region with a CDR2 region that comprises the amino acid sequence of the CDR2 region of the variable heavy chain region of MF6808 (SEQ ID NO: 13); MF6754 (SEQ ID NO: 22); MF6763 (SEQ ID NO: 26); MF6785 (SEQ ID NO: 33); or MF6797 (SEQ ID NO: 51).

In certain aspects, a multispecific antibody or a functional part, derivative and/or analogue thereof according to the use or method of the present disclosure that comprises a variable domain that binds to an extracellular part of CD137 comprises a heavy chain variable region with a CDR1 region that comprises the amino acid sequence of the CDR1 region of the variable heavy chain region of MF6808 (SEQ ID NO: 13); MF6754 (SEQ ID NO: 21); MF6763 (SEQ ID NO: 25); MF6785 (SEQ ID NO: 32); or MF6797 (SEQ ID NO: 50).

In certain aspects, a multispecific antibody or a functional part, derivative and/or analogue thereof according to the use or method of the present disclosure that comprises a variable domain that binds to an extracellular part of CD137 comprises a heavy chain variable region with a CDR1, CDR2 and CDR3 region that comprises the amino acid sequence of the CDR1, CDR2 and CDR3 of a variable heavy chain region of one of the VH presented for MF6808; MF6754; MF6763; MF6785; or MF6797. In certain aspects, the CDR1, CDR2 and CDR3 sequences are selected from the same VH region.

In certain aspects, a multispecific antibody or a functional part, derivative and/or analogue thereof according to the use or method of the present disclosure that comprises a variable domain that binds to an extracellular part of CD137 comprises the amino acid sequence of the variable heavy chain region of MF6808, MF6754; MF6763; MF6785; or MF6797 having at most 15, such as 0, 1, 2, 3, 4, 5, 6, 7, 8, 9 or 10 and such as having 0, 1, 2, 3, 4 or 5 amino acid insertions, deletions, substitutions or a combination thereof with respect to the amino acid sequence of the VH of the indicated ME. In certain aspects, the amino acid sequence of the variable heavy chain region has at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MF. In certain aspects, the amino acid insertion(s), deletion(s), substitution(s) or a combination thereof, if any, are not in the amino acid sequence of the CDR regions.

In certain aspects, the variable domain that binds an extracellular part of CD137 or a functional part, derivative and/or analogue thereof comprised by the multispecific antibody according to the use or method of the present disclosure comprises a VH region with the amino acid sequence of the CDR3 or the amino acid sequence of the CDR1, CDR2 and CDR3 of one of the VH of MF6808; MF6754; MF6763; MF6785; or MF6797. In certain aspects, the variable domain that binds an extracellular part of CD137 comprises a VH region with the amino acid sequence of the VH of MF6808 (SEQ ID NO: 13); MF6754 (SEQ ID NO: 20); MF6763 (SEQ ID NO: 24); MF6785 (SEQ ID NO: 31); or MF6797 (SEQ ID NO: 49) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of the 5 indicated MF. In certain aspects, the amino acid sequence of the variable heavy chain region has at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated ME. In certain aspects, the amino acid insertion(s), deletion(s), substitution(s) or a combination thereof, if any, are not in the amino acid sequence of the CDR regions.

In certain aspects, variable domains comprised by the multispecific antibody or a functional part, derivative and/or analogue thereof according to the use or method of the present disclosure that bind an extracellular part of PD-L1 and that block the binding of PD1 to PD-L1 are variable domains that comprise the amino acid sequence of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5359 (SEQ ID NO: 69); MF5377 (SEQ ID NO: 73); MF5382 (SEQ ID NO: 77); MF5424 (SEQ ID NO: 81); MF5426 (SEQ ID NO: 85); MF5439 (SEQ ID NO: 89); MF5442 (SEQ ID NO: 92); MF5553 (SEQ ID NO: 96); MF5557 (SEQ ID NO: 97); MF5561 (SEQ ID NO: 100); MF5576 (SEQ ID NO: 103); MF5594 (SEQ ID NO: 104); or ME5708 (SEQ ID NO: 107). In certain aspects, the CDRs from said ME sequences are determined using the Kabat numbering system, as mentioned herein elsewhere.

In certain aspects, variable domains comprised by the multispecific antibody or a functional part, derivative and/or analogue thereof according to the use or method of the present disclosure that bind an extracellular part of PD-L1 and that block the binding of PD1 to PD-L1 are variable domains that comprise the CDR1, CDR2 and CDR3 sequences of the amino acid sequence of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5359 (SEQ ID NO: 69); MF5377 (SEQ ID NO: 73); MF5382 (SEQ ID NO: 77); MF5424 (SEQ ID NO: 81); MF5426 (SEQ ID NO: 85); MF5439 (SEQ ID NO: 89); MF5442 (SEQ ID NO: 92); MF5553 (SEQ ID NO: 96); MF5557 (SEQ ID NO: 97); MF5561 (SEQ ID NO: 100); MF5576 (SEQ ID NO: 103); MF5594 (SEQ ID NO: 104); or MF5708 (SEQ ID NO: 107).

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR3 region that comprises the amino acid sequence of the CDR3 region of the variable heavy chain region of MF5554 (SEQ ID NO: 56); MF5576 (SEQ ID NO: 58); MF5578 (SEQ ID NO: 61); MF9375 (SEQ ID NO: 56); MF9376 (SEQ ID NO: 56); MF7702 (SEQ ID NO: 56); MF5424 (SEQ ID NO: 84); MF5561 (SEQ ID NO: 102); MF5439 (SEQ ID NO: 91); MF5553 (SEQ ID NO: 56); MF5594 (SEQ ID NO: 106); MF5426 (SEQ ID NO: 88); or MF5442 (SEQ ID NO: 95).

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR3 region that comprises the amino acid sequence of the CDR3 region of the variable heavy chain region of MF7702 (SEQ ID NO: 56); MF5424 (SEQ ID NO: 84); MF5561 (SEQ ID NO: 102); MF5439 (SEQ ID NO: 91); MF5553 (SEQ ID NO: 56); MF5594 (SEQ ID NO: 106); MF5426 (SEQ ID NO: 88); or MF5442 (SEQ ID NO: 95).

In certain aspects, a multispecific antibody or a functional part, derivative and/or analogue thereof according to the use or method of the present disclosure comprises a heavy chain variable region that binds an extracellular part of PD-L1 comprising a CDR3 region having an amino acid sequence as set forth in SEQ ID NO: 56; SEQ ID NO: 58; SEQ ID NO: 61; SEQ ID NO: 84; SEQ ID NO: 88; SEQ ID NO: 91; SEQ ID NO: 95; SEQ ID NO: 102; or SEQ ID NO: 106. In certain aspects, a multispecific antibody or a functional part, derivative and/or analogue thereof according to the use or method of the present disclosure comprises a heavy chain variable region that binds an extracellular part of PD-L1 comprising a CDR3 region having an amino acid sequence as set forth in SEQ ID NO: 56; SEQ ID NO: 91; SEQ ID NO: 95; or SEQ ID NO: 102, or variants thereof.

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR2 region that comprises the amino acid sequence of the CDR2 region of the variable heavy chain region of MF5554 (SEQ ID NO: 55); MF5576 (SEQ ID NO: 55); MF5578 (SEQ ID NO: 3); MF9375 (SEQ ID NO: 63), MF9376 (SEQ ID NO: 66); MF7702 (SEQ ID NO: 55); MF5424 (SEQ ID NO: 83); MF5561 (SEQ ID NO: 101); MF5439 (SEQ ID NO: 79); MF5553 (SEQ ID NO: 55); MF5594 (SEQ ID NO: 105); MF5426 (SEQ ID NO: 87); or MF5442 (SEQ ID NO: 94).

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR2 region that comprises the amino acid sequence of the CDR2 region of the variable heavy chain region of MF7702 (SEQ ID NO: 56); MF5424 (SEQ ID NO: 84); MF5561 (SEQ ID NO: 102); MF5439 (SEQ ID NO: 91); MF5553 (SEQ ID NO: 56); MF5594 (SEQ ID NO: 106); MF5426 (SEQ ID NO: 88); or MF5442 (SEQ ID NO: 95).

In certain aspects, a multispecific antibody or a functional part, derivative and/or analogue thereof according to the use or method of the present disclosure comprises a variable domain that binds to an extracellular part of PD-L1, wherein the antibody comprises a heavy chain variable region that binds an extracellular part of PD-L1 comprising a CDR2 region having an amino acid sequence as set forth in SEQ ID NO: 3; SEQ ID NO: 55; SEQ ID NO: 63; SEQ ID NO: 66; SEQ ID NO: 79; SEQ ID NO: 83; SEQ ID NO: 87; SEQ ID NO: 94; SEQ ID NO: 101; or SEQ ID NO: 105, or variants thereof.

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR1 region that comprises the amino acid sequence of the CDR1 region of the variable heavy chain region of MF5554 (SEQ ID NO: 54); MF5576 (SEQ ID NO: 54); MF5578 (SEQ ID NO: 60); MF9375 (SEQ ID NO: 60); MF9376 (SEQ ID NO: 65); MF7702 (SEQ ID NO: 68); MF5424 (SEQ ID NO: 82); MF5561 (SEQ ID NO: 93); MF5439 (SEQ ID NO: 90); MF5553 (SEQ ID NO: 68); MF5594 (SEQ ID NO: 74); MF5426 (SEQ ID NO: 86); or MF5442 (SEQ ID NO: 93).

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR1 region that comprises the amino acid sequence of the CDR1 region of the variable heavy chain region of MF7702 (SEQ ID NO: 56); MF5424 (SEQ ID NO: 84); MF5561 (SEQ ID NO: 102); MF5439 (SEQ ID NO: 91); MF5553 (SEQ ID NO: 56); MF5594 (SEQ ID NO: 106); MF5426 (SEQ ID NO: 88); or MF5442 (SEQ ID NO: 95). In certain aspects, a multispecific antibody or according to the use or method of the present disclosure that comprises a variable domain that binds to an extracellular part of PD-L1 comprises a heavy chain variable region that binds an extracellular part of PD-L1 comprising a CDR1 region having an amino acid sequence as set forth in SEQ ID NO: 54; SEQ ID NO: 60; SEQ ID NO: 65; SEQ ID NO: 68; SEQ ID NO: 74; SEQ ID NO: 82; SEQ ID NO: 86; SEQ ID NO: 90; or SEQ ID NO: 93, or variants thereof.

In certain aspects, a multispecific antibody or a functional part, derivative and/or analogue thereof according to the use or method of the present disclosure that comprises a variable domain that binds to an extracellular part of PD-L1 comprises a heavy chain variable region with a CDR1, CDR2 and CDR3 region that comprises the amino acid sequence of the CDR1, CDR2 and CDR3 of a variable heavy chain region of one of the VH presented for MF5554; MF5576; MF5578; MF9375; MF9376; MF7702; MF5424; MF5561; MF5439; MF5553; MF5594; MF5426; or MF5442. The CDR1, CDR2 and CDR3 sequences are preferably selected from the same VH region.

In certain aspects, a multispecific antibody or a functional part, derivative and/or analogue thereof according to the use or method of the present disclosure that comprises a variable domain that binds to an extracellular part of PD-L1 comprises the amino acid sequence of the variable heavy chain region of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5424 (SEQ ID NO: 81); MF5561 (SEQ ID NO: 100); MF5439 (SEQ ID NO: 89); MF5553 (SEQ ID NO: 96); MF5594 (SEQ ID NO: 104); MF5426 (SEQ ID NO: 85); or MF5442 (SEQ ID NO: 92), having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of the indicated MF. In certain aspects, the amino acid sequence of the variable heavy chain region has at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MF. In certain aspects, the amino acid insertion(s), deletion(s), substitution(s) or a combination thereof, if any, are not in the amino acid sequence of the CDR regions.

In certain aspects, a particularly preferred combination in the multispecific antibody or functional part, derivative and/or analogue according to the use or method of the present disclosure is the combination of variable domains that comprise the indicated sequence or variant thereof of MF6797 (SEQ ID NO: 49) and MF7702 (SEQ ID NO: 67); MF6763 (SEQ ID NO: 24) and MF7702 (SEQ ID NO: 67); MF6785 (SEQ ID NO: 31) and MF7702 (SEQ ID NO: 67); MF6797 (SEQ ID NO: 49) and MF5553 (SEQ ID NO: 96); MF6763 (SEQ ID NO: 24) and MF5553 (SEQ ID NO: 96); MF6785 (SEQ ID NO: 31) and MF5553 (SEQ ID NO: 96); MF6754 (SEQ ID NO: 20) and MF5424 (SEQ ID NO: 81); MF6763 (SEQ ID NO: 24) and MF5561 (SEQ ID NO: 100); MF6785 (SEQ ID NO: 31) and MF5439 (SEQ ID NO: 89); MF6754 (SEQ ID NO: 20) and MF5553 (SEQ ID NO: 96); MF6744 (SEQ ID NO: 28) and MF5594 (SEQ ID NO: 104); or MF6783 (SEQ ID NO: 1) and MF5594 (SEQ ID NO: 104).

In certain aspects, a multispecific antibody or a functional part, derivative and/or analogue thereof according to the use or method of the present disclosure comprises:
a CD137 binding variable domain that comprises a VH region with the amino acid sequence of the CDR3 or the amino acid sequence of the CDR1, CDR2 and CDR3 of the VH of MF6797 (SEQ ID NO: 49); and
a PD-L1 binding variable domain that comprises a VH region with the amino acid sequence of the CDR3 or the amino acid sequence of the CDR1, CDR2 and CDR3 of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5594 (SEQ ID NO: 104); MF5424 (SEQ ID NO: 81); MF5426 (SEQ ID NO: 85); MF5553 (SEQ ID NO: 96); MF5442 (SEQ ID NO: 92); MF5561 (SEQ ID NO: 100); or MF5439 (SEQ ID NO: 89).

In certain aspects, a multispecific antibody or a functional part, derivative and/or analogue thereof according to the use or method of the present disclosure comprises:
a CD137 binding variable domain that comprises a VH region with the amino acid sequence of the VH of MF6797 (SEQ ID NO: 49) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the amino acid sequence of the VH of MF6797 (SEQ ID NO: 49) and;
a PD-L1 binding variable domain that comprises a VH region with the amino acid sequence of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5594 (SEQ ID NO: 104), MF5424 (SEQ ID NO: 81); MF5426 (SEQ ID NO: 85); MF5553 (SEQ ID NO: 96); MF5442 (SEQ ID NO: 92); MF5561 (SEQ ID NO: 100); or MF5439 (SEQ ID NO: 89), having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of the indicated ME. In certain aspects, the amino acid sequence of the variable heavy chain regions have at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MIFs.

Certain aspects further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6763 (SEQ ID NO: 27); and
a PD-L1 binding variable domain that comprises a VH region with the amino acid sequence of the CDR3 region of the VH of MF5442 (SEQ ID NO: 95).

Certain aspects provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6763 (SEQ ID NO: 24); and
a PD-L1 binding variable domain that comprises a VH region with the amino acid sequence of the CDR1, CDR2 and CDR3 regions of the VH of MF5442 (SEQ ID NO: 92).

Certain aspects provide a multispecific antibody or a functional part, derivative and/or analogue thereof according to the use or method of the present disclosure that comprises:
a CD137 binding variable domain that comprises a VH region with the amino acid sequence of the VH of MF6763 (SEQ ID NO: 24) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the amino acid sequence of the VH of MF6763; and
a PD-L1 binding variable domain that comprises a VH region with the amino acid sequence of the VH of MF5442 (SEQ ID NO: 92) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of MF5442. In certain aspects, the amino acid sequence of the variable heavy chain regions have at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MFs.

Certain aspects further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6797 (SEQ ID NO: 52); and
a PD-L1 binding variable domain that comprises a VH region with the amino acid sequence of the CDR3 region of the VH of MF7702 (SEQ ID NO: 56).

Certain aspects provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6797 (SEQ ID NO: 49); and
a PD-L1 binding variable domain that comprises a VH region with the amino acid sequence of the CDR1, CDR2 and CDR3 regions of the VH of MF7702 (SEQ ID NO: 67).

Certain aspects provide a multispecific antibody or a functional part, derivative and/or analogue thereof according to the use or method of the present disclosure that comprises:
a CD137 binding variable domain that comprises a VH region with the amino acid sequence of the VH of MF6797 (SEQ ID NO: 49) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the amino acid sequence of the VH of MF6797 and;
a PD-L1 binding variable domain that comprises a VH region with the amino acid sequence of the VH of MF7702 (SEQ ID NO: 67) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of MF7702. In certain aspects, the amino acid sequence of the variable heavy chain regions have at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MFs.

Certain aspects further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6754 (SEQ ID NO: 23); and
a PD-L1 binding variable domain that comprises a VH region with the amino acid sequence of the CDR3 region of the VH of MF5561 (SEQ ID NO: 102).

Certain aspects provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6754 (SEQ ID NO: 20); and
a PD-L1 binding variable domain that comprises a VH region with the amino acid sequence of the CDR1, CDR2 and CDR3 regions of the VH of MF5561 (SEQ ID NO: 100).

Certain aspects provide a multispecific antibody or a functional part, derivative and/or analogue thereof according to the use or method of the present disclosure that comprises:
a CD137 binding variable domain that comprises a VH region with the amino acid sequence of the VH of MF6754 (SEQ ID NO: 20) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the amino acid sequence of the VH of MF6754 and;
a PD-L1 binding variable domain that comprises a VH region with the amino acid sequence of the VH of MF5561 (SEQ ID NO: 100) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of MF5561. In certain aspects, the amino acid sequence of the variable heavy chain regions have at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MFs.

Certain aspects further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6785 (SEQ ID NO: 34); and
a PD-L1 binding variable domain that comprises a VH region with the amino acid sequence of the CDR3 region of the VH of MF5439 (SEQ ID NO: 91).

Also provided is a bispecific antibody or a functional part, derivative and/or analogue thereof that comprises:
a CD137 binding variable domain that comprises a VH region with the amino acid sequence of the CDR1, CDR2 and CDR3 regions of the VH of MF6785 (SEQ ID NO: 31); and
a PD-L1 binding variable domain that comprises a VH region with the amino acid sequence of the CDR1, CDR2 and CDR3 regions of the VH of MF5439 (SEQ ID NO: 89).

Certain aspects provide a multispecific antibody or a functional part, derivative and/or analogue thereof according to the use or method of the present disclosure that comprises:
a CD137 binding variable domain that comprises a VH region with the amino acid sequence of the VH of MF6785 (SEQ ID NO: 31) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the amino acid sequence of the VH of MF6785; and
a PD-L1 binding variable domain that comprises a VH region with the amino acid sequence of the VH of MF5439 (SEQ ID NO: 89) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of MF5439. In certain aspects, the amino acid sequence of the variable heavy chain regions have at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MFs.

Certain aspects further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6785 (SEQ ID NO: 34); and
a PD-L1 binding variable domain that comprises a VH region with the amino acid sequence of the CDR3 region of the VH of MF5542 (SEQ ID NO: 95).

Certain aspects provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6785 (SEQ ID NO: 31); and
a PD-L1 binding variable domain that comprises a VH region with the amino acid sequence of the CDR1, CDR2 and CDR3 regions of the VH of MF5442 (SEQ ID NO: 92).

Certain aspects provide a multispecific antibody or a functional part, derivative and/or analogue thereof according to the use or method of the present disclosure that comprises:
a CD137 binding variable domain that comprises a VH region with the amino acid sequence of the VH of MF6785 (SEQ ID NO: 31) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the amino acid sequence of the VH of MF6785; and
a PD-L1 binding variable domain that comprises a VH region with the amino acid sequence of the VH of MF5442 (SEQ ID NO: 92) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of MF5542. In certain aspects, the amino acid sequence of the variable heavy chain regions have at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MFs.

Certain aspects provide a multispecific antibody or a functional part, derivative and/or analogue thereof according to the use or method of the present disclosure that comprises a binding domain that binds CD137, comprising
a variable domain that comprises a CDR1 according to SEQ ID NO: 50, a CDR2 according to SEQ ID NO: 51 and a CDR3 according to SEQ ID NO: 52, each CDR1, CDR2, and/or CDR3 having 0, 1, 2, 3, 4 or 5 amino acid insertions, deletions, substitutions or a combination thereof, or
a variable domain that comprises a CDR1 according to SEQ ID NO: 40, a CDR2 according to SEQ ID NO: 41 and a CDR3 according to SEQ ID NO: 42, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 21, a CDR2 according to SEQ ID NO: 22 and a CDR3 according to SEQ ID NO: 23, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 32, a CDR2 according to SEQ ID NO: 33 and a CDR3 according to SEQ ID NO: 34, each CDR1, CDR2 and/or CDR3 having 0, 1, 2, 3, 4 or 5 amino acid insertions, deletions, substitutions or a combination thereof; and/or a binding domain that binds PD-L1, comprising
a variable domain that comprises a CDR1 according to SEQ ID NO: 68, a CDR2 according to SEQ ID NO: 55 and a CDR3 according to SEQ ID NO: 56, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 93, a CDR2 according to SEQ ID NO: 94 and a CDR3 according to SEQ ID NO: 95, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 93, a CDR2 according to SEQ ID NO: 101 and a CDR3 according to SEQ ID NO: 102, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 90, a CDR2 according to SEQ ID NO: 79 and a CDR3 according to SEQ ID NO: 91, each CDR1, CDR2 and/or CDR3 having 0, 1, 2, 3, 4 or 5 amino acid insertions, deletions, substitutions or a combination thereof. In certain aspects, the amino acid sequence of the CDRs have at most 5, such as 0, 1, 2, 3, or 4; such as 0, 1, 2, or 3, such as 0, 1 or 2; or such as 0 or 1, amino acid insertions, deletions, substitutions or a combination thereof.

In certain aspects, a multispecific antibody or a functional part, derivative and/or analogue thereof according to the use or method of the present disclosure comprises a variable domain that binds to an extracellular part of CD137 that blocks the binding of CD137 to CD137 ligand and a variable domain that binds to an extracellular part of PD-L1 that blocks the binding of PD-1 to PD-L1. In certain aspects, the variable domain that binds an extracellular part of PD-L1 in this antibody or a functional part, derivative and/or analogue thereof comprises a VH region with the amino acid sequence of the CDR3 or the amino acid sequence of the CDR1, CDR2 and CDR3 of one of the VH of MF5554; MF5576; MF5578; MF9375; MF9376; MF7702; MF5424; MF5561; MF5439; MF5553; MF5594; MF5426; MF5442. In certain aspects, the variable domain that binds an extracellular part of PD-L1 comprises a VH region with the amino acid sequence of a VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5424 (SEQ ID NO: 81); MF5561 (SEQ ID NO: 100); MF5439 (SEQ ID NO: 89); MF5553 (SEQ ID NO: 96); MF5594 (SEQ ID NO: 104); MF5426 (SEQ ID NO: 85); MF5442 (SEQ ID NO: 92) having at most 15, such as 0, 1, 2, 3, 4, 5, 6, 7, 8, 9 or 10 or such as having 0, 1, 2, 3, 4 or 5 amino acid insertions, deletions, substitutions or a combination thereof with respect to the amino acid sequence of the VH of the indicated MF. In certain aspects, the amino acid sequence of the variable heavy chain region has at most 5, such as 0, 1, 2, 3, or 4; such as 0, 1, 2, or 3, such as 0, 1 or 2; or such as 0 or 1, amino acid insertions, deletions, substitutions or a combination thereof with respect to the amino acid sequence of the VH of the indicated MFs.

It is shown in the Examples of WO2018/056821 A1, binding of the CD137-specific VH of MF6797 (SEQ ID NO: 49), is associated with the presence of amino acids comprising Arg66, Gly70 and Phe72 of the CD137 amino acid sequence.

In certain aspects, the present disclosure therefore also provides the use or method of treatment of an isolated, synthetic or recombinant antibody, or a functional part, derivative and/or analogue thereof, that is able to bind to CD137, wherein the binding of said antibody or functional part, derivative or analogue to CD137 is associated with the presence of amino acids comprising Arg66, Gly70 and Phe72 of the CD137 amino acid sequence (SEQ ID NO: 117). In certain aspects, the binding of said antibody or functional part, derivative or analogue to CD137 is also associated with an amino acid comprising Val71 of the CD137 amino acid sequence.

The term “Arg66” refers to the arginine residue at position 66 of the CD137 sequence. The term “Gly70” refers to the glycine residue at position 70 of the CD137 sequence according to SEQ ID NO 117. The term “Val71” refers to the valine residue at position 71 of the CD137 sequence. The term “Phe72” refers to the phenylalanine residue at position 72 of the CD137 sequence.

In certain aspects, the multispecific antibody of the present disclosure comprises binding sites comprising a common light chain according to SEQ ID NO: 109 with 0-5 amino acid insertions, deletions, substitutions, additions or a combination thereof. In certain aspects, each of the binding sites comprises a common light chain according to SEQ ID NO: 109 with 0-5 amino acid insertions, deletions, substitutions, additions or a combination thereof.

In certain aspects, the multispecific antibody of the present disclosure comprises binding sites comprising the LCDR sequences, according to IMGT, of a common light chain according to SEQ ID NO: 109 with 0-5 amino acid insertions, deletions, substitutions, additions or a combination thereof. In certain aspects, each of the binding sites comprises the LCDR sequences, according to IMGT, of a common light chain according to SEQ ID NO: 109 with 0-5 amino acid insertions, deletions, substitutions, additions or a combination thereof. In certain aspects, said substitutions are conservative substitutions whereby amino acids are substituted for a conservative amino acid. A skilled person is well capable of substituting one or more amino acids in the CDR sequence according to the present disclosure. For instance, conservative amino acid substitution is applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine. In certain aspects, the multispecific antibody of the present disclosure comprises binding sites comprising the LCDR sequences (according to the IMGT database worldwide web at imgt.org) of a common light chain according to SEQ ID NO: 109 with 1 or 2 amino acid insertions, deletions, substitutions, additions or a combination thereof. In certain aspects, each of the binding sites comprises the LCDR sequences, according to IMGT, of a common light chain according to SEQ ID NO: 109 with 1 or 2 amino acid insertions, deletions, substitutions, additions or a combination thereof.

In certain aspects, the light chain variable region of one, two or more binding domains of a multispecific antibody of the present disclosure comprises an LCDR1 comprising the amino acid sequence QSISSY, an LCDR2 comprising the amino acid sequence AAS, and an LCDR3 comprising the amino acid sequence QQSYSTPPT (i.e., the CDRs of IGKV1-39 according to IMGT).

In certain aspects, one, two or more binding domains of the multispecific antibody of the present disclosure comprises a light chain variable region comprising an amino acid sequence that is at least 90%, in certain aspects at least 95%, in certain aspects at least 97%, in certain aspects at least 98%, in certain aspects at least 99% identical or in certain aspects 100% identical to the amino acid sequence of SEQ ID NO:109.

In certain aspects, multispecific antibodies as described herein comprise a common light chain variable domain, such as a common light chain variable region according to SEQ ID NO: 110 with 0-5 amino acid insertions, deletions, substitutions, additions or a combination thereof, such as with 0, 1, 2, 3, or 4; or such as with 0, 1, 2, or 3, or such as with 0, 1 or 2; or such as with 0 or 1, amino acid insertions, deletions, substitutions or a combination thereof. In certain aspects, multispecific antibodies as described herein comprise a common light chain constant domain, such as a common light chain constant region according to SEQ ID NO: 111 with 0-5 amino acid insertions, deletions, substitutions, additions or a combination thereof. The term ‘common light chain’ according to the present disclosure refers to light chains which may be identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common LC’, ‘cLC’, ‘single light chain’ with or without the addition of the term ‘rearranged’ are all used herein interchangeably. The terms ‘common light chain variable region’, ‘common VL’, ‘common LCv’, ‘cLCv’, ‘single VL’ with or without the addition of the term ‘rearranged’ are all used herein interchangeably. In certain aspects of the present disclosure, the multispecific antibody has a common light chain (variable region) that can combine with at least two, or such as a plurality of heavy chains (variable regions) of different binding specificity to form antibodies with functional antigen binding domains (WO2004/009618, WO2009/157771). In certain aspects, the common light chain (variable region) is a human light chain (variable region). In certain aspects, a common light chain (variable region) has a germline sequence. In certain aspects, the germline sequence is a light chain variable region that is frequently used in the human repertoire and has good thermodynamic stability, yield and solubility. In certain aspects, the common light chain is the rearranged germline human kappa light chain IgVκ1-39*01/IGJκ1*01 (SEQ ID NO 109). In certain aspects, the common light chain variable region is the variable region of the rearranged germline human kappa light chain IgVκ1-39*01/IGJκ1*01. In certain aspects, the common light chain comprises a light chain variable region as presented in SEQ ID NO 110 with 0-5 amino acid insertions, deletions, substitutions, additions or a combination thereof, such as with 0, 1, 2, 3, or 4; such as with 0, 1, 2, or 3, such as with 0, 1 or 2; or such as with 0 or 1, amino acid insertions, deletions, substitutions or a combination thereof. In certain aspects, the common light further comprises a light chain constant region, such as a kappa light chain constant region. A nucleic acid that encodes the common light chain can be codon optimized for the cell system used to express the common light chain protein. The encoding nucleic acid can deviate from a germ-line nucleic acid sequence.

In certain aspects, the multispecific antibody according to the use or method of the present disclosure is a full length antibody or a fragment of an antibody, for example a Fab fragment or a single-chain variable fragment (scFv). In certain aspects, the multispecific antibody according to the use or method of the present disclosure is a full length antibody.

In certain aspects, the multispecific antibody according to the use or method of the present disclosure is an IgG. In certain aspects, the multispecific antibody is an IgG1 molecule without Fc effector function.

An Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function. Reduced effector functions are preferred in the present disclosure. Reduced effector function can be desired when an immune response is to be activated, enhanced or stimulated as in some of the aspects of the present disclosure. Antibodies with reduced effector functions can be used to target cell-surface molecules of immune cells, among others. In certain aspects, the CH2 region of the multispecific antibody of the present disclosure is engineered to reduce ADCC and/or CDC activity of the antibody. In certain aspects, the CH3 region of the multispecific antibody is engineered to facilitate heterodimerization of the heavy chains.

Antibodies with reduced effector functions are preferably IgG antibodies comprising a modified CH2/lower hinge region, for instance to reduce Fc-receptor interaction or to reduce C1q binding. In certain aspects the antibody of the present disclosure is an IgG antibody with a mutant CH2 and/or lower hinge domain such that interaction of the multispecific IgG antibody to an Fc-gamma receptor is reduced. In certain aspects, CH2 region of the present disclosure comprises an amino acid sequence according to SEQ ID NO: 114 with 0-5 amino acid insertions, deletions, substitutions, additions or a combination thereof. In certain aspects, a hinge region of the present disclosure comprises an amino acid sequence according to SEQ ID NO: 113 with 0-5 amino acid insertions, deletions, substitutions, additions or a combination thereof.

In certain aspects, the CH3 region of the multispecific antibody is engineered to facilitate heterodimerization of the heavy chains. In certain aspects, these variations are present to produce essentially only multispecific full length IgG molecules with amino acid substitutions at positions 351 and 366, e.g., L351K and T366K (numbering according to EU numbering) in the first CH3 domain (the ‘KK-variant’ heavy chain) and amino acid substitutions at positions 351 and 368, e.g., L351D and L368E in the second CH3 domain (the ‘DE-variant’ heavy chain), or vice versa. Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains. In certain aspects, the multispecific antibody of the present disclosure comprises CH3 domains according to SEQ ID NOs 115 and 116 with 0-5 amino acid insertions, deletions, substitutions, additions or a combination thereof, with the proviso that the DE/KK variants are not changed.

In certain aspects, the PD-1 or PD-L1 inhibitor is an antibody or other binding molecule.

In certain aspects, the PD-1 or PD-L1 inhibitor is a PD-1 inhibitor. In certain aspects, the PD-1 inhibitor is selected from Nivolumab, Pembrolizumab, Cemiplimab, Penpulimab, Retifanlimab, Sintilimab, Tislelizumab, Toripalimab and Dostarlimab. In certain aspects, the PD-1 inhibitor is Pembrolizumab. In certain aspects, the PD-1 or PD-L1 inhibitor is a PD-L1 inhibitor. In certain aspects, the PD-L1 inhibitor is selected from Atezolizumab, Avelumab, and Durvalumab. The amino acid sequences of Nivolumab, Pembrolizumab, Cemiplimab, Dostarlimab, Atezolizumab, Avelumab, and Durvalumab are well known in the art.

In certain aspects, the PD-1 or PD-L1 inhibitor is administered pursuant to the present recommendation set by FDA approval.

In certain aspects, the PD-1 inhibitor is Nivolumab and administered in an amount of 3 mg/kg as an intravenous infusion over (e.g. over 60 minutes) every 2 weeks until disease progression or unacceptable toxicity.

In an alternative aspect, Nivolumab is administered at 240 mg every 2 weeks or 480 mg every 4 weeks, or 1 mg/kg followed by ipilimumab 3 mg/kg on the same day every 3 weeks for 4 doses, then 240 mg every 2 weeks or 480 mg every 4 weeks.

In an alternative aspect, Nivolumab is administered at 3 mg/kg every 2 weeks with ipilimumab 1 mg/kg every 6 weeks or at 360 mg every 3 weeks with ipilimumab 1 mg/kg every 6 weeks and 2 cycles of platinum-doublet chemotherapy.

In an alternative aspect, Nivolumab is administered at 360 mg every 3 weeks with ipilimumab 1 mg/kg every 6 weeks.

In an alternative aspect, Nivolumab is administered at 240 mg every 2 weeks or 480 mg every 4 weeks, or at 3 mg/kg every 2 weeks, or at 3 mg/kg followed by ipilimumab 1 mg/kg on the same day every 3 weeks for 4 doses, then 240 mg every 2 weeks or 480 mg every 4 weeks.

In certain aspects, the PD-1 inhibitor is Cemiplimab and is administered in an amount of 350 mg as an intravenous infusion over (e.g. over 30 minutes) every 3 weeks until disease progression or unacceptable toxicity.

In certain aspects, the PD-1 inhibitor is Dostarlimab and is administered as a first dose through a fourth dose of 500 mg every 3 weeks, followed by subsequent dosing beginning 3 weeks after dose 4 (dose 5 onwards): 1,000 mg every 6 weeks, as an intravenous infusion over (e.g. over 30 minutes) until disease progression or unacceptable toxicity.

In certain aspects, the PD-L1 inhibitor is Atezolizumab and is administered in an amount of 1200 mg as an intravenous infusion over (e.g. over 60 minutes) every 3 weeks until disease progression or unacceptable toxicity. If the first infusion is tolerated, all subsequent infusions may be delivered over 30 minutes.

In certain aspects, the PD-L1 inhibitor is Avelumab and is administered in an amount of 10 mg/kg as an intravenous infusion over (e.g. over 60 minutes) every 2 weeks until disease progression or unacceptable toxicity.

In certain aspects, the PD-L1 inhibitor is Durvalumab and is administered in an amount of 10 mg/kg administered as an intravenous infusion (e.g. over 60 minutes) every 2 weeks until disease progression or unacceptable toxicity.

In certain aspects, the PD-1 inhibitor is Pembrolizumab and administered in an amount of 400 mg as an intravenous infusion or administered at 2 mg/kg as an intravenous infusion every 3 weeks. In certain aspects, the administration is a flat dose of 400 mg and occurs every 6 weeks until disease progression or unacceptable toxicity.

In certain aspects, the PD-1 inhibitor is Pembrolizumab and administered in an amount of 400 mg as an intravenous infusion every 6 weeks. In certain aspects, the PD-1 inhibitor is Pembrolizumab and administered as a flat dose in an amount of 400 mg as an intravenous infusion every 6 weeks. In certain aspects, the PD-1 inhibitor is Pembrolizumab and is administered in an amount of 200 mg as an intravenous infusion every 3 weeks. In certain aspects, the PD-1 inhibitor is Pembrolizumab and is administered as flat dose in an amount of 200 mg as an intravenous infusion every 3 weeks. In certain aspects, the administration is a flat dose of 400 mg and occurs every 6 weeks until disease progression or unacceptable toxicity. In certain aspects, the administration is a flat dose of 200 mg and occurs every 3 weeks until disease progression or unacceptable toxicity.

The term “flat dose” herein refers to a dosing regimen wherein a subject is administered with a fixed amount of a therapeutic substance over multiple administrations, independent of body weight of the subject. Flat dosing is typically abbreviated with qnw, wherein n is an integer indicating the interval and w is week. For instance, a q6w flat dose administration regimen of 400 mg antibody means a fixed amount of 400 mg antibody is administered each 6 weeks. In certain aspects, Pembrolizumab is administered with a q6w dosing regimen of 400 mg. In certain aspects, Pembrolizumab is administered with a q3w dosing regimen of 200 mg.

The flat dose may be premedicated, meaning medication is administered to the subject prior to being administered the antibody of the present disclosure. In certain aspects, the Pembrolizumab or multispecific antibody flat dose of the disclosure is premedicated with an antihistamine, pain reducing medication, fever reducing medication and/or anti-inflammatory medication.

In certain aspects, the PD-1 or PD-L1 inhibitor is a full length antibody or a fragment of an antibody, for example a Fab fragment or a single-chain variable fragment (scFv). In certain aspects, the PD-1 or PD-L1 inhibitor according to the use or method of the present disclosure is a full length antibody. In certain aspects, the PD-1 or PD-L1 inhibitor is, or comprises, an antibody selected from Nivolumab, Pembrolizumab, Cemiplimab, Dostarlimab, Atezolizumab, Avelumab, and Durvalumab or a functional fragment or variant thereof.

In certain aspects, the PD-1 or PD-L1 inhibitor comprises or consists of an amino acid sequence that is or has substantial sequence identity to the amino acid sequence of the antigen binding site of one of Nivolumab, Pembrolizumab, Cemiplimab, Dostarlimab, Atezolizumab, Avelumab, and Durvalumab, in particular Pembrolizumab.

In certain aspects, the PD-1 or PD-L1 inhibitor is, or comprises, at least one, such as at least two, even such as three CDRs that are identical or substantially identical to the CDRs of one of Nivolumab, Pembrolizumab, Cemiplimab, Dostarlimab, Atezolizumab, Avelumab, and Durvalumab, in particular Pembrolizumab. In certain aspects, the PD-1 or PD-L1 inhibitor comprises, at least one, such as at least two, even such as three CDRs that are identical to the CDRs found in Pembrolizumab.

In certain aspects, the PD-1 or PD-L1 inhibitor is, or comprises, a CDR1 that is identical to a CDR1 from one of Nivolumab, Pembrolizumab, Cemiplimab, Dostarlimab, Atezolizumab, Avelumab, and Durvalumab, a CDR2 that is identical to a CDR2 from one of Nivolumab, Pembrolizumab, Cemiplimab, Dostarlimab, Atezolizumab, Avelumab, and Durvalumab, or a CDR3 that is identical to a CDR3 from one of Nivolumab, Pembrolizumab, Cemiplimab, Dostarlimab, Atezolizumab, Avelumab, and Durvalumab, in certain aspects a CDR1 and a CDR2 that are identical to a CDR1 and a CDR2 from one of Nivolumab, Pembrolizumab, Cemiplimab, Dostarlimab, Atezolizumab, Avelumab, and Durvalumab, a CDR1 and a CDR3 that are identical to a CDR1 and a CDR3 from one of Nivolumab, Pembrolizumab, Cemiplimab, Dostarlimab, Atezolizumab, Avelumab, and Durvalumab, or a CDR2 and a CDR3 that are identical to a CDR2 and a CDR3 from one of Nivolumab, Pembrolizumab, Cemiplimab, Dostarlimab, Atezolizumab, Avelumab, and Durvalumab, even such as a CDR1, a CDR2 and a CDR3 that are identical to a CDR1, a CDR2 and a CDR3 from one of Nivolumab, Pembrolizumab, Cemiplimab, Dostarlimab, Atezolizumab, Avelumab, and Durvalumab. In certain aspects, the PD-1 or PD-L1 inhibitor comprises CDR1, CDR2 and CDR3 that are identical to CDR1, CDR2 and CDR3 from Pembrolizumab.

In certain aspects, the multispecific antibody is administered in a dose of between 10-300 mg, such as between 10-150 mg or 25-100 mg, such as between 25-75 mg. In certain aspects, the multispecific antibody is administered in a dose of 25, 30, 35, 40, 45, or 50 mg. In certain aspects, the multispecific antibody is administered in a flat dose of 25, 30, 35, 40, 45, or 50 mg.

In certain aspects, the multispecific antibody is administered around every 7, 10, 11, 12, 13, 14, 15, 16, 17 or 18, or 21 days, particularly around every 10, 14 or 18 days, more particularly around every 14 days.

In certain aspects, the PD-1 or PD-L1 inhibitor is administered in a dose pursuant to its approved dose amount and schedule as approved by the USFDA. In certain aspects, Pembrolizumab is administered in an amount of between about 300-500 mg, such as between 350-450 mg, such as between 375-425 mg. In certain aspects, the PD-1 or PD-L1 inhibitor is administered in a dose of 325, 350, 375, 400, 425, 450 or 475 mg.

In some aspects, the multispecific antibody is administered in a dose of 10 mg, 15 mg, 20 mg, 25 mg, 30 mg, 35 mg, 40 mg, 45 mg, 50 mg, 75 mg, 100 mg, 125 mg or 150 mg.

In certain aspects, the multispecific antibody according to the use or method of the present disclosure is administered in a flat dose.

In certain aspects, the multispecific antibody according to the use or method of the present disclosure is administered in a dose, or flat dose, of between 50-150 mg, such as between 75-150 mg, such as between 25-125 mg or 100-150 mg. In certain aspects, the multispecific antibody according to the use or method of the present disclosure is administered in a dose, or flat dose, of between 50-150 mg, or between 75-150 mg, or between 75-125 mg or between 100-150 mg.

In certain aspects, the multispecific antibody according to the use or method of the present disclosure is administered in a dose, or flat dose, of between 75-125 mg.

In certain aspects, the multispecific antibody according to the use or method of the present disclosure is administered in a dose, or flat dose, of between 50-150 mg, such as between 75-125 mg. In certain aspects, the multispecific antibody according to the use or method of the present disclosure is administered in a dose, or flat dose, of between 50-100 mg, or between 75-100 mg or between 75-125 mg.

In certain aspects, the multispecific antibody according to the use or method of the present disclosure is administered in a dose, or flat dose, of between 25-75 mg.

In certain aspects, the multispecific antibody according to the use or method of the present disclosure is administered in a dose, or flat dose, of between 10-50 mg, such as between 25-50 mg. In certain aspects, the multispecific antibody according to the use or method of the present disclosure is administered in a dose, or flat dose, of between 10-50 mg, or between 25-50 mg.

In certain aspects, the multispecific antibody according to the use or method of the present disclosure is administered once every week, once every two weeks or once every three weeks. In certain aspects, the multispecific antibody according to the use or method of the present disclosure is administered once every two weeks. In certain aspects, if the multispecific antibody according to the use or method of the present disclosure is administered once every week, the multispecific antibody is administered in a dose of between 10-100 mg, such as between 15-75 mg. In certain aspects, if the multispecific antibody according to the use or method of the present disclosure is administered once every week, the multispecific antibody is administered in a dose of between 10-100 mg, or between 15-75 mg, such as between 15-50 mg, or between 15-40 mg, or between 15-30 mg, or between 15-25 mg.

In certain aspects, the multispecific antibody according to the use or method of the present disclosure is administered in a flat dose once every two weeks.

In certain aspects, the multispecific antibody according to the use or method of the present disclosure is administered in a dose, or flat dose of 25, 30, 35, 40, 45, or 50 mg once every two weeks. In certain aspects, the multispecific antibody according to the use or method of the present disclosure is administered in a dose, or flat dose of 25 mg once every two weeks. In certain aspects, the multispecific antibody according to the use or method of the present disclosure is administered in a dose, or flat dose of 40 mg once every two weeks. In certain aspects, the multispecific antibody according to the use or method of the present disclosure is administered in a dose, or flat dose of 50 mg once every two weeks.

In certain aspects, the PD-1 inhibitor is Pembrolizumab and administered in an amount of 200 mg as an intravenous infusion every 3 weeks and the treatment further comprises administering of a multispecific antibody of the present disclosure in an amount of 10 mg once every two weeks. In certain aspects, the PD-1 inhibitor is Pembrolizumab and administered in an amount of 200 mg as an intravenous infusion every 3 weeks and the treatment further comprises administering of a multispecific antibody of the present disclosure in an amount of 15 mg once every two weeks. In certain aspects, the PD-1 inhibitor is Pembrolizumab and administered in an amount of 200 mg as an intravenous infusion every 3 weeks and the treatment further comprises administering of a multispecific antibody of the present disclosure in an amount of 25 mg once every two weeks. In certain aspects, the PD-1 inhibitor is Pembrolizumab and administered in an amount of 200 mg as an intravenous infusion every 3 weeks and the treatment further comprises administering of a multispecific antibody of the present disclosure in an amount of 40 mg once every two weeks.

In certain aspects, the PD-1 inhibitor is Pembrolizumab and administered in an amount of 400 mg as an intravenous infusion every 6 weeks and the treatment further comprises administering of a multispecific antibody of the present disclosure in an amount of 10 mg once every two weeks. In certain aspects, the PD-1 inhibitor is Pembrolizumab and administered in an amount of 400 mg as an intravenous infusion every 6 weeks and the treatment further comprises administering of a multispecific antibody of the present disclosure in an amount of 15 mg once every three weeks. In certain aspects, the PD-1 inhibitor is Pembrolizumab and administered in an amount of 400 mg as an intravenous infusion every 6 weeks and the treatment further comprises administering of a multispecific antibody of the present disclosure in an amount of 25 mg once every two weeks. In certain aspects, the PD-1 inhibitor is Pembrolizumab and administered in an amount of 400 mg as an intravenous infusion every 6 weeks and the treatment further comprises administering of a multispecific antibody of the present disclosure in an amount of 40 mg once every two weeks.

In certain aspects, the PD-1 inhibitor is Pembrolizumab and administered in an amount of 200 mg as an intravenous infusion every 3 weeks and the treatment further comprises administering of a multispecific antibody of the present disclosure in an amount of 10 mg once every three weeks. In certain aspects, the PD-1 inhibitor is Pembrolizumab and administered in an amount of 200 mg as an intravenous infusion every 3 weeks and the treatment further comprises administering of a multispecific antibody of the present disclosure in an amount of 15 mg once every three weeks. In certain aspects, the PD-1 inhibitor is Pembrolizumab and administered in an amount of 200 mg as an intravenous infusion every 3 weeks and the treatment further comprises administering of a multispecific antibody of the present disclosure in an amount of 25 mg once every three weeks. In certain aspects, the PD-1 inhibitor is Pembrolizumab and administered in an amount of 200 mg as an intravenous infusion every 3 weeks and the treatment further comprises administering of a multispecific antibody of the present disclosure in an amount of 40 mg once every three weeks.

In certain aspects, the PD-1 inhibitor is Pembrolizumab and administered in an amount of 400 mg as an intravenous infusion every 6 weeks and the treatment further comprises administering of a multispecific antibody of the present disclosure in an amount of 10 mg once every three weeks. In certain aspects, the PD-1 inhibitor is Pembrolizumab and administered in an amount of 400 mg as an intravenous infusion every 6 weeks and the treatment further comprises administering of a multispecific antibody of the present disclosure in an amount of 15 mg once every three weeks. In certain aspects, the PD-1 inhibitor is Pembrolizumab and administered in an amount of 400 mg as an intravenous infusion every 6 weeks and the treatment further comprises administering of a multispecific antibody of the present disclosure in an amount of 25 mg once every three weeks. In certain aspects, the PD-1 inhibitor is Pembrolizumab and administered in an amount of 400 mg as an intravenous infusion every 6 weeks and the treatment further comprises administering of a multispecific antibody of the present disclosure in an amount of 40 mg once every three weeks.

In certain aspects, the multispecific antibody according to the use or method of the present disclosure is administered intravenously.

In certain aspects, the multispecific antibody according to the use or method of the present disclosure is administered over a period of between 30 minutes to 4 hours, such as between 1 to 3 hours, or such as 2 hours.

In certain aspects, the multispecific antibody according to the use or method of the present disclosure is administered over a period of between 30 minutes to 4 hours, such as between 1 to 3 hours, or such as 2 hours.

In certain aspects, the multispecific antibody according to the use or method of the present disclosure is administered intravenously as a flat dose over a period of 2 hours every 14 days in 28-day cycles.

In certain aspects, the multispecific antibody according to the use or method of the present disclosure is administered intravenously as a (flat) dose of 10 mg over a period of 2 hours every 14 days in 28-day cycles. In certain aspects, the multispecific antibody according to the use or method of the present disclosure is administered intravenously as a (flat) dose of 15 mg over a period of 2 hours every 14 days in 28-day cycles. In certain aspects, the multispecific antibody according to the use or method of the present disclosure is administered intravenously as a (flat) dose of 25 mg over a period of 2 hours every 14 days in 28-day cycles. In certain aspects, the multispecific antibody according to the use or method of the present disclosure is administered intravenously as a (flat) dose of 30 mg over a period of 2 hours every 14 days in 28-day cycles. In certain aspects, the multispecific antibody according to the use or method of the present disclosure is administered intravenously as a (flat) dose of 40 mg over a period of 2 hours every 14 days in 28-day cycles. In certain aspects, the multispecific antibody according to the use or method of the present disclosure is administered intravenously as a (flat) dose of 50 mg over a period of 2 hours every 14 days in 28-day cycles. In certain aspects, the multispecific antibody according to the use or method of the present disclosure is administered intravenously as a (flat) dose of 60 mg over a period of 2 hours every 14 days in 28-day cycles. In certain aspects, the multispecific antibody according to the use or method of the present disclosure is administered intravenously as a (flat) dose of 70 mg over a period of 2 hours every 14 days in 28-day cycles. In certain aspects, the multispecific antibody according to the use or method of the present disclosure is administered intravenously as a (flat) dose of 75 mg over a period of 2 hours every 14 days in 28-day cycles.

In certain aspects, the multispecific antibody according to the use or method of the present disclosure is formulated as a liquid in a concentration of 1 mg/mL to 100 mg/mL, such as at or around 20 mg/mL. In certain aspects, the multispecific antibody according to the use or method of the present disclosure is formulated as a liquid in a concentration of 1 mg/mL to 100 mg/mL, or at or around 20 mg/mL.

In certain aspects, Pembrolizumab is administered at a dose, or a flat dose, of between 100-500 mg, or 100-300 mg or 300-500 mg, or 150-250 mg or 350-450 mg, or 175-225 mg or 375-425 mg.

In certain aspects, the PD-1 or PD-L1 inhibitor is administered at a dose, or a flat dose, of around 150 mg, 175 mg, 200 mg, 225 mg, 250 mg, 275 mg, 300 mg, 325 mg, 350 mg, 375 mg, 400 mg, 425 mg, 450 mg or 475 mg, or at around 175 mg, 200 mg, 225 mg, 375 mg, 400 mg, or 425 mg.

In certain aspects, the PD-1 or PD-L1 inhibitor, such as Pembrolizumab, is administered in once every three weeks. In certain aspects, the PD-1 or PD-L1 inhibitor, such as Pembrolizumab, is administered in once every six weeks.

In certain aspects, Pembrolizumab is administered at a dose or flat dose of between 100-300 mg, or 150-250 mg, or 175-225 mg, once every three weeks. In certain aspects, Pembrolizumab is administered at a dose or flat dose of between 300-500 mg, or 350-450 mg, or 375-425 mg, once every six weeks.

In certain aspects, the PD-1 or PD-L1 inhibitor is Pembrolizumab and is administered at a dose, or a flat dose, of around 150 mg, 175 mg, 200 mg, 225 mg, 250 mg, every 21 days. In certain aspects, the PD-1 or PD-L1 inhibitor is Pembrolizumab and is administered at a dose, or a flat dose, of around 350 mg, 375 mg, 400 mg, 425 mg, or 450 mg, every 42 days.

In certain aspects, the PD-inhibitor is first administered around 21 days after the first administration of the multispecific antibody. In certain aspects, the PD-inhibitor is first administered around 42 days after the first administration of the multispecific antibody.

In certain aspects, the PD-1 or PD-L1 inhibitor is Pembrolizumab and is administered at a dose, or a flat dose, of around 150 mg, 175 mg, 200 mg, 225 mg, 250 mg, every 21 days. In certain aspects, the PD-1 or PD-L1 inhibitor is Pembrolizumab and is administered at a dose, or a flat dose, of around 350 mg, 375 mg, 400 mg, 425 mg, or 450 mg, every 42 days. In certain aspects, the PD-1 or PD-L1 inhibitor is Pembrolizumab and is administered at a dose, or a flat dose, of around 200 mg every 21 days. In certain aspects, the PD-1 or PD-L1 inhibitor is Pembrolizumab and is administered at a dose, or a flat dose, of around 400 mg every 42 days.

In certain aspects, the PD-1 or PD-L1 inhibitor is administered via IV, particularly IV infusion. Such an infusion may be administered over, for example 15, 30 or 45 minutes.

In certain aspects, the PD-1 or PD-L1 inhibitor is Pembrolizumab and is administered at a dose, or a flat dose, of around 150 mg, 175 mg, 200 mg, 225 mg, 250 mg, every 21 days, via IV infusion. In certain aspects, the PD-1 or PD-L1 inhibitor is Pembrolizumab and is administered at a dose, or a flat dose, of around 350 mg, 375 mg, 400 mg, 425 mg, or 450 mg, every 42 days, via IV infusion.

In certain aspects, the multispecific antibody is administered as a dose or flat dose of 10 mg, 25 mg, 30 mg, 40 mg or 50 mg every 14 days and the PD-1 or PD-L1 inhibitor is Pembrolizumab and is administered at a dose, or a flat dose, of around 150 mg, 175 mg, 200 mg, 225 mg, 250 mg, every 21 days. In certain aspects, the multispecific antibody is administered as a dose or flat dose of 10 mg, 25 mg, 30 mg, 40 mg or 50 mg every 14 days and the PD-1 or PD-L1 inhibitor is Pembrolizumab and is administered at a dose, or a flat dose, of around 350 mg, 375 mg, 400 mg, 425 mg, or 450 mg, every 42 days.

In certain aspects, the multispecific antibody is administered as a flat dose of 10 mg, 15 mg, 25 mg or 40 mg every 14 days and the PD-1 or PD-L1 inhibitor is Pembrolizumab and is administered at a flat dose of around 200 mg every 21 days. In certain aspects, the multispecific antibody is administered as a flat dose of 10 mg, 15 mg, 25 mg or 40 mg every 14 days and the PD-1 or PD-L1 inhibitor is Pembrolizumab and is administered as a flat dose of around 400 mg every 42 days.

In certain aspects, the multispecific antibody is administered as a flat dose of 10 mg, 15 mg, 25 mg or 40 mg every 21 days and the PD-1 or PD-L1 inhibitor is Pembrolizumab and is administered at a flat dose of around 200 mg every 21 days. In certain aspects, the multispecific antibody is administered as a flat dose of 10 mg, 15 mg, 25 mg or 40 mg every 21 days and the PD-1 or PD-L1 inhibitor is Pembrolizumab and is administered as a flat dose of around 400 mg every 42 days.

In certain aspects, the multispecific antibody is administered as a dose or flat dose of 40 mg, every 14 days and the PD-1 or PD-L1 inhibitor is Pembrolizumab and is administered at a dose, or a flat dose, of around 200 mg, every 21 days or at a dose, or a flat dose, of around 400 mg, every 42 days.

In certain aspects, the multispecific antibody is administered as a dose or flat dose of 40 mg, every 21 days and the PD-1 or PD-L1 inhibitor is Pembrolizumab and is administered at a dose, or a flat dose, of around 200 mg, every 21 days or at a dose, or a flat dose, of around 400 mg, every 42 days.

In certain aspects, the multispecific antibody is administered as a dose or flat dose of 25 mg, every 14 days and the PD-1 or PD-L1 inhibitor is Pembrolizumab and is administered at a dose, or a flat dose, of around 200 mg, every 21 days or at a dose, or a flat dose, of around 400 mg, every 42 days.

In certain aspects, the multispecific antibody is administered as a dose or flat dose of 30 mg, every 14 days and the PD-1 or PD-L1 inhibitor is Pembrolizumab and is administered at a dose, or a flat dose, of around 200 mg, every 21 days or at a dose, or a flat dose, of around 400 mg, every 42 days.

In certain aspects, the multispecific antibody is administered as a dose or flat dose of 40 mg, every 14 days and the PD-1 or PD-L1 inhibitor is Pembrolizumab and is administered at a dose, or a flat dose, of around 200 mg, every 21 days or at a dose, or a flat dose, of around 400 mg, every 42 days.

In certain aspects, the multispecific antibody is administered as a dose or flat dose of 50 mg, every 14 days and the PD-1 or PD-L1 inhibitor is Pembrolizumab and is administered at a dose, or a flat dose, of around 200 mg, every 21 days or at a dose, or a flat dose, of around 400 mg, every 42 days.

In certain aspects, the PD-1 or PD-L1 inhibitor is formulated as a liquid in a concentration of 1 mg/mL to 100 mg/mL, or at or around 25 mg/mL.

In certain aspects, the multispecific antibody according to the use or method of the present disclosure is administered prior to, simultaneously, or such as after, an antihistamine, a nonsteroidal anti-inflammatory drug (NSAID), a narcotic, an intravenous fluid, an antipyretic, a bronchodilator, oxygen, a corticosteroid (IV/oral), a vasopressor, or any combination thereof, is administered to reduce infusion-related reactions. Premedication with the indicated substances may be opted for subjects in case an infusion-related reaction is experienced to prevent and mitigate the incidence and severity thereof.

In certain aspects, the multispecific antibody according to the use or method of the present disclosure is administered after the subject has been pretreated with standard of care therapy, such as chemotherapy, immunotherapy or targeted therapy, for advanced metastatic disease.

In certain aspects, the multispecific antibody according to the use or method of the present disclosure is administered to a subject that has not been treated with an anti-PD-L1 agent, such as an anti-PD-L1 antibody, or T-cell agonist.

In certain aspects, the cancer is an advanced or metastatic solid tumor. In particular the cancer may be selected from lung cancer, particularly locally advanced or metastatic NSCLC, or melanoma, especially such cancers that have previously been treated with immune checkpoint therapies and/or that are positive for PD-L1. In certain aspects, said cancer is an advanced or metastatic solid tumor which has a PD-L1 expression of 1% or higher. In certain aspects, said cancer or subject comprising said cancer either has received prior anti-PD-1/PD-L1 approved therapy immunotherapy and has a cancer having a PD-L1 expression of 1% or higher or said cancer or subject is treatment naïve and has a cancer having a PD-L1 expression of 1% or higher.

In certain aspects, the cancer is an advanced or metastatic solid tumor, such as selected from locally advanced or metastatic lung cancer and locally advanced or metastatic melanoma. In certain aspects, said melanoma is selected from cutaneous, acral or mucosal melanoma. In certain aspects, the cancer is a Merkel cell carcinoma. Merkel cell carcinoma, also referred to herein as neuroendocrine carcinoma of the skin or trabecular cancer, is a very rare type of skin cancer that forms when Merkel cells grow out of control and form a cancer or tumor.

PD-L1 expression is determined by clinicians of ordinary skill in the art, see exemplary methods in de Ruiter et al. (2021), incorporated herewith in its entirety. High expression as determined by any one of these methods shall constitute high expression for purposes of this disclosure. When evaluated by tumor proportion score (TPS), a TPS score of greater than or equal to 1% is a PD-L1 positive cancer. When evaluated by combined positive score (CPS), a score of greater than or equal to 1% is a PD-L1 positive cancer. A cancer is PD-L1 high if having a PD-L1 expression score of 10% or higher, 20% or higher, 30% or higher, 40% or higher, or 50% or higher when determined using the tumor proportion score (TPS). A cancer is PD-L1 high if having a PD-L1 expression score of 5% or higher, 15% or higher, or 20% or higher, when determined using the combined positive score (CPS). If evaluated by multiple such methods, if any one or more method identifies a tumor as PD-L1 positive, it meets the criteria for the present disclosure.

In certain aspects, the subject has not received prior treatment with an immune checkpoint inhibitor. In certain aspects, the subject has not received prior treatment with an immune checkpoint inhibitor and their cancer is positive for PD-L1 expression. In certain aspects, the subject has not received prior treatment with an immune checkpoint inhibitor and is being treated for melanoma, particularly locally advanced or metastatic melanoma. In certain aspects, the subject has not received prior treatment with an immune checkpoint inhibitor and is being treated for lung cancer, in particular non-small cell lung cancer (NSCLC).

The NSCLC may be non-squamous or squamous. In some aspects, the subject may have had treatments based on BRAF, ALK, EGFR and/or ROS1 status. In certain aspects the subject may have progressed on or be intolerable to such treatments. In certain aspects, the NSCLC is unresectable.

In certain aspects, the melanoma is cutaneous, acral or mucosal melanoma. Melanomas may be confirmed by standard histological or cytologic processes. In certain aspects, the melanoma is not ocular or uveal melanoma. In certain aspects, the melanoma is Stage III or Stage IV melanoma (as per standard staging systems, such as the American Joint Staging Committee). It may be unresectable and/or not amenable to local therapy.

In certain aspects, the melanoma may be BRAF positive. In those aspects, the subject may have received BRAF±NMEF targeted therapy, and particularly may have shown progression.

In certain aspects, the subject has received prior standard therapy, particularly systemic therapy, for advanced or recurrent/metastatic disease or may be intolerant to such therapy. Standard therapy may include, for example, chemotherapy, immunotherapy, and targeted regimens. The subject may have received, for example, 1, 2, 3, 4 or 5, but particularly 4 or fewer regimens.

In certain aspects, the subject has received prior immune checkpoint therapy, particularly PD-L1/PD-1 therapy. In some such aspects, the subject has received no more than one prior PD-1 therapy for advanced or metastatic cancers. In some such aspects, the subject may have relapsed following previous therapy, for example their disease has progressed or, at best, remained stable after at least 6 months of treatment. In certain aspects, the subject has not prior treatment with an immune checkpoint inhibitor.

In some aspects, the subject may have received monotherapy with the multispecific antibody for 1, 2, 3, 4, 5, or 6 months, particularly 1 or 2 months, prior to beginning combination therapy.

In certain aspects, the subject is an adult. In certain aspects, the subject is mammalian, especially a human.

In certain aspects, the subject does not have one or more conditions selected from Burkitt lymphoma, lymphoblastic leukemia/lymphoma, lymphoplasmacytic lymphoma, chronic lymphocytic leukemia, CNS metastases/lymphoma, carcinomatous meningitis, autoimmune disease, active HBV, active HCV, HIV. In certain aspects, the subject has not received prior therapies containing a 4-1BB agonist, CAR T-cell therapy or a solid organ or allogeneic stem cell transplant. In certain aspects, the subject does not have a history of idiopathic pulmonary fibrosis, interstitial lung disease, organizing pneumonia), drug-induced pneumonitis or idiopathic pneumonitis, or active pneumonitis.

In certain aspects, the multispecific antibody according to the use or method of the present disclosure increases the number of CD8⁺ T cells, in particular in breast cancer, more in particular in immunodeficient mice bearing human MDA-MB-231 tumors.

In certain aspects, the multispecific antibody according to the use or method of the present disclosure is significantly less toxic than a combination of urelumab and atezolizumab, such as determined in the same study, in particular in breast cancer, more in particular in immunodeficient mice bearing human MDA-MB-231 tumors.

In certain aspects, the multispecific antibody according to the use or method of the present disclosure does not induce graft-versus-host disease.

The term “antibody” as used herein means a proteinaceous molecule, such as belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Antibodies such as the multispecific antibodies of the present disclosure typically comprise the constant domains (Fc part) of a natural antibody, which may be engineered as described elsewhere herein, for instance to reduce ADCC and/or CDC activity.

A “multispecific antibody” refers to an antibody comprising at least two binding sites with different antigen or epitope specificity. In certain aspects, one or more of the antigen binding sites comprises an immunoglobulin VH/VL pair. In certain aspects, each of the antigen binding sites comprises an immunoglobulin VH/VL pair.

In certain aspects, a multispecific antibody according to the present disclosure has no more than two antigen binding sites. This means that the antigen binding part of such multispecific antibody consists of two antigen binding sites, without the presence of additional antigen binding sites. In certain aspects, each of the two antigen binding sites contains an immunoglobulin VH/VL pair.

In certain aspects, the VL in each VH/VL pair is similar. In certain aspects, the VL in each VH/VL pair is identical. In certain aspects, the multispecific antibody is a full length antibody which has one heavy/light (H/L) chain combination that binds an extracellular part of CD137 and one H/L chain combination that binds an extracellular part of a member of the B7 family. In certain aspects, the light chain in said first H/L chain combination is similar to the light chain in said second H/L chain combination. In certain aspects, the light chains in the first and second H/L chain combinations are identical.

In certain aspects, the multispecific antibody is a bispecific antibody.

The term ‘bispecific antibody’ means that one part of the antibody binds to one epitope on an antigen whereas a second part binds to a different epitope on either the same antigen, or a different antigen. The different epitopes are typically present on different antigens. The different epitopes can, however, also be present on the same antigen. Dependent on the expression level, (sub-)cellular localization and stoichiometry of the two antigens recognized by a bispecific antibody, both Fab arms of the antibody may or may not simultaneously bind their epitope. One arm of the bispecific antibody typically contains the variable domain of one antibody and the other arm contains the variable domain of another antibody (i.e. one arm of the bispecific antibody is formed by one heavy chain paired with one light chain whereas the other arm is formed by a different heavy chain paired with a light chain). In certain aspects, the heavy chain variable regions of a bispecific antibody of the present disclosure are different from each other, whereas the light chain variable regions are the same in the bispecific antibodies of the present disclosure. A bispecific antibody wherein the different heavy chain variable regions are associated with the same or a common, light chain variable region is also referred to as a bispecific antibody with a common light chain variable region (cLcv). In certain aspects, the light chain constant region is also the same. Such bispecific antibodies are referred to as having a common light chain (cLc).

Certain preferred aspects are immunoglobulins having an IgG format, providing the advantage that the half-lives of bivalent binding molecules/antibodies/variants according to the disclosure are typically longer as compared to multivalent compounds. Moreover, the immunogenicity of bivalent binding molecules according to the disclosure is typically lower as compared to multivalent compounds. Molecules/antibodies/variants according to these aspects preferably maintain the structure of natural IgGs and therefore maintain all benefits associated to that structure of natural IgGs.

A “variant” of an antibody or multispecific antibody as described herein comprises a functional part, derivative and/or analogue of the antibody or multispecific antibody. The variant may be a fragment of an antibody for example a Fab fragment. The variant may be a single-chain variable fragment (scFv). The variant maintains the binding specificity of the antibody. The functional part, derivative and/or analogue maintains the binding specificity of the antibody. Binding specificity is defined by capacity to bind an extracellular part of a first membrane protein and a second membrane protein as described herein. A variant may have amino acid insertions, deletions, substitutions, or a combination thereof relative to a given amino acid sequence (e.g. any SEQ ID NO. of the present disclosure), at most 15, such as 0, 1, 2, 3, 4, 5, 6, 7, 8, 9 or 10, such as 0, 1, 2, 3, 4 or 5, such as 0, 1, 2, 3, or 4; such as 0, 1, 2, or 3, such as 0, 1 or 2; or such as 0 or 1 amino acid insertions, deletions, substitutions or a combination thereof with respect to the amino acid sequence of a given SEQ ID NO.

As used herein, the term “antigen binding site” means a site of a binding molecule or antibody that specifically binds an epitope of an antigen. Such antigen binding site is preferably derived from or shares sequence homology with the variable domain of an antibody, in particular the CDR regions thereof. In some preferred aspects, said antigen binding site is an immunoglobulin variable domain, formed by an immunoglobulin VH/VL pair. In other aspects, said antigen binding site is derived from an antibody mimetic, such as for instance from an affibody molecule, affilin, affimer, affitin, alphabody, anticalin, avimer, DARPin, fynomer, kunitz domain peptide or monobody, which are described herein before.

The term ‘full length’ according to the disclosure is defined as comprising an essentially complete antibody, without one or more artificially added moieties with a size of larger than 20 amino acid residues, such as for instance additional antigen binding sites or additional activation sites or additional ligands or additional ligand binding moieties. A full length antibody however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH for the heavy chain, and CL, VL for the light chain. The domains of the heavy chains are preferably present in the order of a natural antibody (VHCH1-CH2-CH3; meaning that the VH domain is adjacent to the CH1 domain, followed by a CH2 domain and subsequently followed by a CH3 domain). The domains of the light chains are also preferably present in the order of a natural antibody (VL-CL; meaning that the VL domain is adjacent to the CL domain). An antibody binds to antigen via the variable domains contained in the Fab fragment portion. The antibody can interact with molecules and cells of the immune system through the constant domains, mostly through the Fc portion.

In certain aspects, full length IgG antibodies are preferred because of their typically favorable half-life and the desire to stay as close to fully autologous (human) molecules for reasons of immunogenicity. In certain aspects, a multispecific antibody of the present disclosure is a full length IgG1, a full length IgG2, a full length IgG3 or a full length IgG4 antibody.

Full length antibodies encompass antibodies wherein mutations may be present that provide desired characteristics or are just alternatives to the ones in the original chain. Such mutations typically are not deletions of substantial portions of any of the regions. However, antibodies wherein one or several amino acid residues are inserted, deleted, substituted or a combination thereof, without essentially altering the antigen binding characteristics of the resulting antibody are embraced within the term “full length antibody”. For instance, an IgG antibody can have 1-20 amino acid residue insertions, substitutions, deletions or a combination thereof in the constant region.

Note that in the present specification, unless stated otherwise, amino acid positions assigned to CDRs and frameworks in a heavy chain variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

In certain aspects, “percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The alignment may also be carried out over individual CDR sequences. The sequence identity is the percentage of identical matches between the two sequences over the reported aligned region.

In certain aspects, a comparison of sequences and determination of percentage of sequence identity between two sequences is accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. In certain aspects, the NEEDLE program from the EMBOSS package is used to determine percent identity of amino acid and nucleic acid sequences (version 2.8.0, EMBOSS: The European Molecular Biology Open Software Suite (2000) Rice, P. LongdenJ. and Bleasby, A. Trends in Genetics 16, (6) pp 276 277, http://emboss.bioinformatics.nl/). In certain aspects, for protein sequences, EBLOSUM62 is used for the substitution matrix. In certain aspects, DNAFULL is used for DNA sequences. The parameters used are a gap-open penalty of 10 and a gap extension penalty of 0.5.

After alignment by the program NEEDLE as described above the percentage of sequence identity between a query sequence and a sequence of this disclosure is calculated as follows: Number of corresponding positions in the alignment showing an identical amino acid or identical nucleotide in both sequences divided by the total length of the alignment after subtraction of the total number of gaps in the alignment.

In certain aspects, at the start of treatment, at least one, more than one or all of the following inclusion factors IF1-IF20 are applicable to subjects for treatment. With start of treatment is meant the first administration of the multispecific antibody of the present disclosure. In certain aspects, the subject comprises or complies with all of IF1-IF:
IF1. Having a Life expectancy of ≥12 weeks.
IF2. Having an ECOG performance status of 0 or 1.
IF3. Having a locally advanced tumors relapsed to PD-L1/PD-1 and positive (≥1%) for PD-L1 expression on tumor and/or tumor-associated immune cells (based on local testing after completion of most recent prior line of therapy)
IF4. Having measurable disease per RECIST v1.1 or Lugano Criteria.
IF5. Having received prior standard therapy for advanced or recurrent/metastatic disease as applicable to tumor type, are intolerant to treatment, or refuse standard treatment.
IF6. Having received a maximum of 4 prior systemic treatment regimens (inclusive of chemotherapy, immunotherapy, and targeted therapy regimens) for advanced or recurrent/metastatic disease unless approved by the medical monitor.
IF7. Having received a maximum of 1 prior anti-PD-1 therapy containing immunotherapy regimen in the advanced/metastatic setting, with the proviso that anti-PD-1 therapy in combination with chemotherapies, targeted therapies, or other immunotherapies is permitted.

### Tumor-Specific Criteria

IF8. Subjects with histologically or cytologically confirmed melanoma, having histologically or cytologically confirmed cutaneous, acral, or mucosal melanoma.
IF9. Unresectable Stage III or Stage IV melanoma per American Joint Committee on Cancer staging system that is not amenable to local therapy.
IF10. Having a V600-activating BRAF mutation status or consent to BRAF testing during the screening period if previously not tested.
IF11. If BRAF mutation-positive, must have received BRAF±MEK targeted therapy with documented progression.
IF12. Relapsed (eg, progressed after more than 6 months of treatment with at least a stable disease as best response) to PD-L1/PD-1 therapies
IF13. Relapsed (eg, progressed after more than 6 months of treatment with at least a stable disease as best response) to PD-L1/PD-1 therapies
IF14. Naïve from PD-L1/PD-1 therapies
IF15. Subjects having documented progression on anti-PD-1 therapy as defined by meeting one of the criteria below:
   IF15a. Primary refractory: Having received prior treatment of an anti-PD-1 therapy (alone or as part of a combination) in the advanced or metastatic setting for a minimum of 12 weeks and have PD as their best response to treatment.
   IF15b. Secondary resistance: Having received prior anti-PD-1 therapy (alone or as part of a combination) in the advanced or metastatic setting and achieved CR, PR, or SD but later had confirmed PD while receiving anti-PD-1 therapy (PD confirmed at least 4 weeks [no less than 28 days] later).
IF16. Subjects with histologically or cytologically confirmed NSCLC having histologically or cytologically confirmed diagnosis of NSCLC (either nonsquamous or squamous).
   IF16a. Documentation of test results for mutations or gene rearrangements for EGFR, ALK, BRAF, and ROS1 if the tumor is of nonsquamous histology only, whereas if mutations or gene arrangements are present, subjects have progressed on or be intolerable to targeted therapy.
IF17. Unresectable advanced or metastatic NSCLC as per AJCC staging system that is not amenable to local therapy.
IF18. Subjects having relapsed (eg, progressed after more than 6 months of treatment with at least a stable disease as best response) to PD-L1/PD-1 therapies
IF19. Subjects having either relapsed (eg, progressed after more than 6 months of treatment with at least a stable disease as best response) to PD-L1/PD-1 therapies, or being naïve from PD-L1/PD-1 therapies.
IF20. Subjects having documented progression on anti-PD-1 therapy as defined by meeting one of the following criteria:
   IF20a. For primary refractory, subjects having received prior anti-PD-1 therapy (alone or as part of a combination) in the advanced or metastatic setting for a minimum of 12 weeks and have PD as their best response to treatment.
   IF20b. For secondary resistance, subjects having received prior anti-PD-1 therapy (alone or as part of a combination) in the advanced or metastatic setting and achieved CR, PR, or SD but later had confirmed PD while receiving anti-PD-1 therapy (PD confirmed at least 4 weeks [no less than 28 days] later).

In certain aspects, the subject for treatment complies with any one or more factors selected from the group consisting of IF1-IF20.

In certain aspects, at the start of treatment, at least one, more than one or all of the following exclusion factors EF1-EF23 are applicable to subjects for treatment:
EF1. Having had treatment with anticancer medications or investigational drugs within the following intervals before administration of the first dose of the multispecific antibody of the present disclosure of:
   EF1a: at least 14 days for chemotherapy (6 weeks for mitomycin C and nitrosoureas), targeted small molecule therapy, or radiation therapy, wherein subjects have not had radiation pneumonitis as a result of treatment and a one-week washout period is permitted for palliative radiation to non-CNS disease.
   EF1b: at least 14 or 28 days for a prior antibody used for anticancer therapy,
   EF1c: for other agents with long half-lives (e.g., >5 days), administration of the multispecific antibody of the present disclosure is before the fifth half-life requires medical monitor approval,
   EF1d: at least 10 weeks for radioimmunotherapy, or
   EF1e: subjects not having had an allogeneic SCT within the last 6 months or an autologous SCT within the last 3 months.
EF2: Subjects not having recovered to ≤Grade 1 or baseline from toxic effects of prior therapy (including prior immunotherapy) and/or complications from prior surgical intervention before the first administration of the multispecific antibody of the present disclosure, with the proviso that alopecia and stable neuropathy (≤Grade 2) are allowed.
EF3. Subjects with a liver toxicity of Grade 1 or higher from prior anti-PD-1 therapy (±anti-CTLA-4 therapy), with the proviso that baseline transaminases and bilirubin are out of normal limits.
EF4. Subjects with prior ≥Grade 3 immune-mediated adverse events with anti-PD-1 therapy, with the proviso that the following Grade 3 or higher adverse events are permitted: Grade 3 rashes that resolved with topical therapy; immune-mediated adrenal insufficiency, Type 1 diabetes mellitus, or other endocrine abnormalities due to previous immunotherapy that are medically stable and adequately managed on a stable dose of replacement therapy; and asymptomatic amylase or lipase elevations that did not require treatment interruption.
EF5. Having a history of any grade immune-mediated ocular adverse events.
EF6. Subjects with laboratory values at screening defined following table.

Exclusionary Laboratory Values

| | |
|---|---|
| Laboratory Parameter | Exclusion Criterion |

Hematology

| | | |
|---|---|---|
| EF6a | Platelets | <100 × 10⁹/L |
| EF6b | Hemoglobin | <9 g/dL or 5.6 mmol/L |
| EF6c | ANC | <1.5 × 10⁹/L |

Hepatic

| | | |
|---|---|---|
| EF6d | ALT | ≥1.5 × ULN |
| EF6e | AST | ≥1.5 × ULN |
| EF6f | Total bilirubin | ≥1.2 × ULN unless conjugated bilirubin is ≤ULN |
| | | (conjugated bilirubin only needs to be tested if total |
| | | bilirubin exceeds the ULN) with the proviso that if |
| | | there is no institutional ULN, then conjugated bilirubin |
| | | is <40% of total bilirubin. |
| EF6g | Alkaline phosphatase | ≥2.5 × ULN |
| | | Participants with bone metastases and no hepatic |
| | | parenchymal metastases on screening radiographic |
| | | examinations may enroll if alkaline phosphatase |
| | | is ≤5.0 × ULN, with the proviso that subjects with bone |
| | | metastases and hepatic parenchymal metastases on |
| | | screening radiographic examinations have alkaline |
| | | phosphatase is ≤5.0 × ULN only with medical |
| | | monitor approval. |

Renal

| | | |
|---|---|---|
| EF6h | Clearance of creatinine | creatinine clearance <30 mL/min |

Coagulation

| | | |
|---|---|---|
| EF6i | International normalized | >1.5 × ULN |
| | ratio or prothrombin time | |
| EF6j | Activated partial | >1.5 × ULN |
| | thromboplastin time | |

Other

| | | |
|---|---|---|
| EF6k | Albumin | <3 g/dL |

EFF7 Subjects having clinically significant cardiac disease, including known history of left ventricular ejection fraction of <50%, unstable angina, acute myocardial infarction within 6 months of Cycle 1 Day 1, New York Heart Association Class III or IV congestive heart failure, or arrhythmia requiring therapy, with the proviso that subjects having an arrhythmia receives antiarrhythmic medication and is in sinus rhythm on the screening ECG.
EF7. Subjects having a history or presence of an ECG that, in the investigator's opinion, is clinically meaningful.
EF8. Subjects having an immune-related toxicity during prior checkpoint inhibitor therapy for which permanent discontinuation of therapy is recommended (per product label or consensus guidelines), or any immune-related toxicity requiring intensive or prolonged immunosuppression to manage (with the exception of endocrinopathy that is well controlled on replacement hormones).
EF9. Subjects having a history of idiopathic pulmonary fibrosis, interstitial lung disease, organizing pneumonia (e.g., bronchiolitis obliterans), drug-induced pneumonitis or idiopathic pneumonitis, or evidence of active pneumonitis on screening chest CT scan.
EF10. Subjects having a history of uncontrolled asthma or chronic obstructive pulmonary disease, with the proviso that having a history of radiation pneumonitis in the radiation field (fibrosis) is permitted.
EF11. Subjects having known active CNS metastases/lymphoma and/or carcinomatous meningitis, with the proviso that subjects with previously-treated brain metastases are permitted if they are EF11a: radiologically stable (ie, without evidence of progression for at least 28 days by repeat imaging prior to the first dose of the multispecific antibody of the present disclosure,
EF11b: without requirement of steroid treatment for at least 14 days prior to first dose of the multispecific antibody of the present disclosure, or EF11c: clinically stable with any neurological signs or symptoms having returned to baseline, with the proviso that carcinomatous meningitis is excluded regardless of clinical stability.
EF12: Subjects with evidence of cerebral edema or those with <28 days since radiation therapy to the CNS.
EF13: Subjects who have active or inactive autoimmune disease or syndrome (eg, rheumatoid arthritis, moderate or severe psoriasis, multiple sclerosis, inflammatory bowel disease) that has required systemic treatment in the past 2 years or who are receiving systemic therapy for an autoimmune or inflammatory disease (ie, with use of disease modifying agents, corticosteroids, or immunosuppressive drugs), with the proviso that subjects with vitiligo, resolved childhood asthma/atopy, hypothyroidism stable on hormone replacement, controlled asthma, Type I diabetes, Graves' disease, or Hashimoto's disease, or with medical monitor approval, are permitted if they meet all other eligibility criteria.
EF14: Subjects using systemic corticosteroids (≥10 mg/day prednisone or equivalent) within 7 days before administration of the first dose of the multispecific antibody of the present disclosure, with the proviso that the use of inhaled or topical corticosteroids or systemic corticosteroids for imaging procedures is permitted and with the proviso that the use of physiologic corticosteroid replacement therapy may be approved after consultation with the medical monitor.
EF15. Receipt of a live vaccine within 30 days before administration of the first dose of the multispecific antibody of the present disclosure or pembrolizumab, including live vaccines, but not limited to, measles, mumps, rubella, chicken pox, yellow fever, rabies, Bacillus Calmette-Guerin, and typhoid vaccine.
EF16. If COVID-19 live vaccines are available, consultation with the medical monitor is required prior to administration.
EF17. Seasonal influenza vaccines for injection are generally killed virus vaccines and are allowed; with the proviso that intranasal influenza vaccines that are live attenuated vaccines are not allowed.
EF18. Having active infection requiring systemic therapy.
EF19. Having a history of solid organ or allogeneic stem cell transplant.
EF20. Having an active HBV or HCV infection that requires treatment, while Hepatitis B virus DNA and HCV RNA must be undetectable, with the proviso that subjects who have cleared a prior HBV infection (defined as hepatitis B surface antigen negative, hepatitis B surface antibody positive, hepatitis B core antibody positive) and subjects with no prior history of HBV infection who have been vaccinated against HBV and who have a positive antibody against hepatitis B surface antigen as the only evidence of prior exposure and subjects that are Hepatitis C antibody-positive who received and completed treatment for hepatitis C that was intended to eradicate the virus may participate if HCV RNA levels are undetectable are permitted.
EF21. Having a known history of HIV (HIV 1/2 antibodies).
EF22. Having a known hypersensitivity or severe reaction to any component of the multispecific antibody of the present disclosure, to Pembrolizumab or to formulation components.
EF23. Being pregnant or breastfeeding or expecting to conceive or father children within the projected duration of the study, starting with the screening visit through 90 days after the last dose of the multispecific antibody of the present disclosure.

In certain aspects, the subject for treatment complies with any one or more factors selected from the group consisting of EF1-EF23. In certain aspects, the subject for treatment complies with all of the factors EF1-EF23.

### CLAUSES

Certain aspects of disclosure are as defined in the following clauses.
1. A multispecific antibody that comprises an antigen binding site that binds an extracellular part of CD137 and an antigen binding site that binds an extracellular part of a second membrane protein for use in a method of treatment of cancer in a subject in need thereof, wherein the treatment further comprises administering of a PD-L1 or PD-1 inhibitor.
2. A method of treating cancer in a subject in need thereof, the method comprising administering of a multispecific antibody that comprises an antigen binding site that binds an extracellular part of CD137 and an antigen binding site that binds an extracellular part of a second membrane protein and a PD-1 or PD-L1 inhibitor to the subject in need thereof.
3. The multispecific antibody for use according to claim 1 or method according to claim 2, wherein the multispecific antibody is administered simultaneously, sequentially, or separately with the PD-1 or PD-L1 inhibitor.
4. The multispecific antibody for use or the method according to any one of the preceding claims, wherein the cancer is an advanced or metastatic solid tumor, such as selected from locally advanced or metastatic lung cancer and locally advanced or metastatic melanoma.
5. The multispecific antibody for use or the method according to any one of the preceding claims, wherein the cancer is NSCLC.
6. The multispecific antibody for use or the method according claim 4, wherein the melanoma is selected from cutaneous, acral or mucosal melanoma.
7. The multispecific antibody for use or the method according to any preceding clause, wherein the cancer is relapsed to a PD-1/PD-L1 therapy and/or is positive for PD-L1 expression.
8. The multispecific antibody for use or method according to any preceding clause, wherein the PD-1 or PD-L1 inhibitor is an antibody, or a variant or functional fragment thereof.
9. The multispecific antibody for use or method according to any preceding clause wherein the PD-1 or PD-L1 inhibitor is a PD-1 inhibitor.
10. The multispecific antibody for use or method according to clause 9, wherein the PD-1 inhibitor is selected from Nivolumab, Pembrolizumab, Cemiplimab, Penpulimab, Retifanlimab, Sintilimab, Tislelizumab, Toripalimab and Dostarlimab.
11. The multispecific antibody for use or method according to clause 10, wherein the PD-1 inhibitor is Pembrolizumab.
12. The multispecific antibody for use or method according to any preceding clause wherein the PD-1 or PD-L1 inhibitor is a PD-L1 inhibitor.
13. The multispecific antibody for use or method according to clause 12, wherein the PD-L1 inhibitor is selected from Atezolizumab, Avelumab, and Durvalumab.
14. The multispecific antibody for use or method according to any preceding clause, wherein the multispecific antibody is administered in a dose of between 10 mg-1200 mg, 15-1200 mg or 25-1200 mg, such as between 10-600 mg, 15-600 mg, 25-600 mg, 10-300 mg, 15-300 mg, or 25-300 mg, such as between 10-150 mg, 10-100 mg, 10-75 mg, 15-150 mg, 15-100 mg, 15-75 mg, 25-150 mg, 25-100 mg or 25-75 mg, or such as between 10-50 mg, 15-50 mg, 25-50 mg or 50-100 mg.
15. The multispecific antibody for use or method according to any preceding clause, wherein the multispecific antibody is administered in a dose of around 10 mg, 15 mg, 20 mg, 25 mg, 30 mg, 35 mg, 40 mg, 45 mg, 50 mg, 60 mg or 75 mg.
16. The multispecific antibody for use or method according to any preceding clause, wherein the PD-1 or PD-L1 inhibitor is administered in a dose of around 300-500 mg, such as 325-475 mg, such as 350-450 mg, or such as 375-425 mg.
17. The multispecific antibody for use or method according to any preceding clause, wherein the multispecific antibody is administered every 14 days.
18. The multispecific antibody for use or method according to any preceding clause, wherein the PD-1 or PD-L1 inhibitor is administered every 4, 5, 6, 7 or 8 weeks.
19. The multispecific antibody for use or method according to clause 18, wherein the PD-1 or PD-L1 inhibitor is administered every 6 weeks.
20. The multispecific antibody for use or method according to any preceding clause, wherein the multispecific antibody and the PD-1 or PD-L1 inhibitor are administered in a dose combination shown in the following table:

| | | | | | |
|---|---|---|---|---|---|
| Around 10 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 15 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 20 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 30 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 40 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 50 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 60 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 75 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 100 mg | Around 10 | Around 10 | Around 10 | Around 10 | Around 10 |
| (multispecific | mg and | mg and | mg and | mg and | mg and |
| antibody) and around | around 125- | around 150- | around 150- | around 150- | around 125- |
| 100-300 mg (PD-1 | 275 mg | 275 mg | 250 mg | 250 mg | 225 mg |
| inhibitor) | | | | | |
| Around 10 mg | Around 10 | Around 10 | Around 10 | Around 10 | Around 10 |
| (multispecific | mg and | mg and | mg and | mg and | mg and |
| antibody) and around | around 325- | around 350- | around 350- | around 350- | around 325- |
| 300-500 mg (PD-1 | 475 mg | 475 mg | 450 mg | 425 mg | 425 mg |
| inhibitor) | | | | | |
| Around 15 mg and | Around 15 | Around 15 | Around 15 | Around 15 | Around 15 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 20 mg and | Around 20 | Around 20 | Around 20 | Around 20 | Around 20 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 25 mg and | Around 25 | Around 25 | Around 25 | Around 25 | Around 25 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 30 mg and | Around 30 | Around 30 | Around 30 | Around 30 | Around 30 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 35 mg and | Around 35 | Around 35 | Around 35 | Around 35 | Around 35 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 40 mg and | Around 40 | Around 40 | Around 40 | Around 40 | Around 40 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 45 mg and | Around 45 | Around 45 | Around 45 | Around 45 | Around 45 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 50 mg and | Around 50 | Around 50 | Around 50 | Around 50 | Around 50 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 55 mg and | Around 55 | Around 55 | Around 55 | Around 55 | Around 55 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 60 mg and | Around 60 | Around 60 | Around 60 | Around 60 | Around 60 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 65 mg and | Around 65 | Around 65 | Around 65 | Around 65 | Around 65 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 70 mg and | Around 70 | Around 70 | Around 70 | Around 70 | Around 70 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 75 mg and | Around 75 | Around 75 | Around 75 | Around 75 | Around 75 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |
| Around 100 mg and | Around 100 | Around 100 | Around 100 | Around 100 | Around 100 |
| around 300-500 mg | mg and | mg and | mg and | mg and | mg and |
| | around 325- | around 350- | around 350- | around 350- | around 325- |
| | 475 mg | 475 mg | 450 mg | 425 mg | 425 mg |

21. The multispecific antibody for use or method according to any preceding clause, wherein the multispecific antibody is administered intravenously.
22. The multispecific antibody for use or method according to any preceding clause, wherein the multispecific antibody is administered once every two weeks.
23. The multispecific antibody for use or method according to any preceding clause, wherein said second membrane protein is not a member of the TNF receptor superfamily.
24. The multispecific antibody for use or method according to any preceding clause, wherein said second membrane protein is a member of the B7 family.
25. The multispecific antibody for use or method according to any preceding clause, wherein the second membrane protein is PD-L1 or PD-L2, such as PD-L1.
26. The multispecific antibody for use or method according to any preceding clause, wherein the multispecific antibody comprises one antigen binding site that binds the PD-1 binding domain of PD-L1.
27. The multispecific antibody for use or method according to any preceding clause, wherein the multispecific antibody comprises one antigen binding site that binds the CD137L binding domain of CD137.
28. The multispecific antibody for use or method according to any preceding clause, wherein the multispecific antibody comprises one antigen binding site that blocks the binding of a ligand to CD137 or binds an extracellular ligand-blocking binding site of CD137, such as a CD137L blocking binding site.
29. The multispecific antibody for use or method according to any preceding clause, wherein the variable domain that binds an extracellular part of CD137 is defined as a variable domain that, when in a bivalent monospecific antibody format that comprises two of said variable domains that bind CD137, does not stimulate activity of CD137 on a cell or does so at a reduced level in comparison to one of said variable domain as part of a bispecific antibody having a second variable domain binding a tumor associated antigen, such as a member of the B7 family, such as PD-L1.
30. The multispecific antibody for use or method according to any preceding clause, wherein the variable domain that binds an extracellular part of CD137 is capable of stimulating activity of CD137 on a cell when combined in a multispecific antibody with a second variable domain which binds to PD-L1, when the multispecific antibody is in the presence of a first cell expressing CD137 and a second cell expressing PD-L1.
31. The multispecific antibody for use or method according to any preceding clause, wherein the multispecific antibody is capable of binding CD137 and PD-L1, such as simultaneously.
32. The multispecific antibody for use or method according to any preceding clause, wherein the multispecific antibody only induces or activates CD137 signaling in the presence of PD-L1 expressing cells.
33. The multispecific antibody for use or method according to any preceding clause, wherein the antigen binding sites of said multispecific antibody consist of one immunoglobulin variable domain that binds CD137 and one immunoglobulin variable domain that binds the extracellular part of a second membrane protein.
34. The multispecific antibody for use or method according to any preceding clause, wherein said multispecific antibody is a full length antibody.
35. The multispecific antibody for use or method according to any preceding clause, wherein said multispecific antibody is an IgG1 molecule without Fc effector function.
36. The multispecific antibody for use or method according to any preceding clause, wherein the second membrane protein is not to a significant extent expressed by a T-cell.
37. The multispecific antibody for use or method according to any preceding clause, wherein said second membrane protein is present on the cell membrane as a part of a multimeric membrane protein comprising two or more of said second membrane proteins.
38. The multispecific antibody for use or method according to any preceding clause, wherein said second membrane protein is present on the cell membrane as a part of a homodimer or a homotrimer.
39. The multispecific antibody for use or method according to any preceding clause, wherein the antibody comprises a heavy chain variable region that binds an extracellular part of CD137 comprising a CDR3 region having an amino acid sequence as set forth in SEQ ID NO: 23; SEQ ID NO: 27; SEQ ID NO: 34 or SEQ ID NO: 52, or variants thereof.
40. The multispecific antibody for use or method according to any preceding clause, wherein the antibody comprises a heavy chain variable region that binds an extracellular part of CD137 comprising a CDR2 region having an amino acid sequence as set forth in SEQ ID NO: 22; SEQ ID NO: 26; SEQ ID NO: 33; or SEQ ID NO: 51, or variants thereof.
41. The multispecific antibody for use or method according to any preceding clause, wherein the antibody comprises a heavy chain variable region that binds an extracellular part of CD137 comprising a CDR1 region having an amino acid sequence as set forth in SEQ ID NO: 21; SEQ ID NO: 25; SEQ ID NO: 32; or SEQ ID NO: 50, or variants thereof.
42. The multispecific antibody for use or method according to any preceding clause, wherein the antibody comprises a heavy chain variable region having an amino acid sequence as set forth in SEQ ID NO: 1; SEQ ID NO: 5; SEQ ID NO: 9; SEQ ID NO: 13; SEQ ID NO: 17; SEQ ID NO: 20; SEQ ID NO: 24; SEQ ID NO: 28; SEQ ID NO: 31; SEQ ID NO: 35; SEQ ID NO: 39; SEQ ID NO: 43; SEQ ID NO: 46; or SEQ ID NO: 49 that binds an extracellular part of CD137, such as SEQ ID NO: 20; SEQ ID NO: 24; SEQ ID NO: 31; or SEQ ID NO: 49, or variants thereof.
43. The multispecific antibody for use or method according to any preceding clause, wherein the antibody comprises a heavy chain variable region comprising a CDR3 region having an amino acid sequence as set forth in SEQ ID NO: 56; SEQ ID NO: 58; SEQ ID NO: 61; SEQ ID NO: 84; SEQ ID NO: 88; SEQ ID NO: 91; SEQ ID NO: 95; SEQ ID NO: 102; or SEQ ID NO: 106 that binds an extracellular part of PD-L1, such as SEQ ID NO: 56; SEQ ID NO: 91; SEQ ID NO: 95; or SEQ ID NO: 102, or variants thereof.
44. The multispecific antibody for use or method according to any preceding clause, wherein the antibody comprises a heavy chain variable region comprising a CDR2 region having an amino acid sequence as set forth in SEQ ID NO: 3; SEQ ID NO: 55; SEQ ID NO: 63; SEQ ID NO: 66; SEQ ID NO: 79; SEQ ID NO: 83; SEQ ID NO: 87; SEQ ID NO: 94; SEQ ID NO: 101; or SEQ ID NO: 105 that binds an extracellular part of PD-L1, or variants thereof
45. The multispecific antibody for use or method according to any preceding clause, wherein the antibody comprises a heavy chain variable region comprising a CDR1 region having an amino acid sequence as set forth in SEQ ID NO: 54; SEQ ID NO: 60; SEQ ID NO: 65; SEQ ID NO: 68; SEQ ID NO: 74; SEQ ID NO: 82; SEQ ID NO: 86; SEQ ID NO: 90; or SEQ ID NO: 93 that binds an extracellular part of PD-L1, or variants thereof.
46. The multispecific antibody for use or method according to any preceding clause, wherein the antibody comprises a heavy chain variable region having an amino acid sequence as set forth in SEQ ID NO: 53; SEQ ID NO: 57; SEQ ID NO: 59; SEQ ID NO: 62; SEQ ID NO: 64; SEQ ID NO: 67; SEQ ID NO: 69; SEQ ID NO: 73; SEQ ID NO: 77; SEQ ID NO: 81; SEQ ID NO: 85; SEQ ID NO: 89; SEQ ID NO: 92; SEQ ID NO: 96; SEQ ID NO: 97; SEQ ID NO: 100; SEQ ID NO: 103; SEQ ID NO: 104; SEQ ID NO: 107 that binds an extracellular part of PD-L1, such as SEQ ID NO: 67, SEQ ID NO: 89, SEQ ID NO: 92, or SEQ ID NO: 100, or variants thereof.
47. The multispecific antibody for use or method according to any preceding clause, wherein the multispecific antibody comprises CDR 1, 2 and 3 of MF6797 and CDRs 1, 2 and 3 of MF7702, or variants thereof.
48. The multispecific antibody for use or method according to any preceding clause, wherein the multispecific antibody comprises SEQ ID NO. 49 and SEQ ID NO. 67, or variants thereof.
49. The multispecific antibody for use or method according to any preceding clause, wherein the cancer is a Merkel Cell Carcinoma.
50. A multispecific antibody for use in a method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof,
wherein the multispecific antibody is for simultaneous, sequential, or separate administration with a PD-1 or PD-L1 inhibitor.
51. The multispecific antibody for use according to clause 49, wherein the multispecific antibody is administered in a dose of between 10-300 mg, 15-300 mg, or 25-300 mg, such as between 25-150 or mg 25-100 mg, such as between 50-100 mg.
52. The multispecific antibody for use according to clause 50 or 51, wherein the cancer is a Merkel Cell Carcinoma.
53. A method of treating cancer in a subject in need thereof, the method comprising administering a multispecific antibody that comprises a binding domain that binds CD137, comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof;
and a PD-1 or PD-L1 inhibitor, to a subject in need thereof.
54. The method according to clause 53, wherein the multispecific antibody is administered in a dose of between 10-300 mg, 25-300 mg, such as between 25-150 or mg 25-100 mg, such as between 50-100 mg.
55. The multispecific antibody for use or method according to any one of clauses 50-54, wherein the cancer is an advanced or metastatic solid tumor.
56 The multispecific antibody for use or the method according to clause 55, wherein the advanced or metastatic solid tumor is selected from: advanced or metastatic lung cancer, in particular non-small cell lung cancer (NSCLC); and advanced or metastatic melanoma.
57. A multispecific antibody for use in a method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof,
wherein the multispecific antibody is for simultaneous, subsequent or separate administration with a PD-1 or PD-L1 inhibitor; and
wherein the cancer is selected an advanced or metastatic solid tumor.
58. The multispecific antibody for use or method according to any one of clauses 50-57, wherein the multispecific antibody is administered intravenously.
59. The multispecific antibody for use or method according to any one of clauses 50-58, wherein the multispecific antibody is administered once every two weeks.
60. The multispecific antibody for use or method according to any one of clauses 50-59, wherein the multispecific antibody comprises CDR 1, 2 and 3 of MF6797 and CDRs 1, 2 and 3 of MF7702, or variants thereof.
61. The multispecific antibody for use or method according to clause 60, wherein the multispecific antibody comprises SEQ ID NO. 49 and SEQ ID NO. 67, or variants thereof
62. The multispecific antibody for use or method according to any one of clauses 50-59, wherein the multispecific antibody comprises CDR 1, 2 and 3 of MF6783 and CDRs 1, 2 and 3 of MF5542, or variants thereof.
63. The multispecific antibody for use or method according to clause 62, wherein the multispecific antibody comprises SEQ ID NO. 1 and SEQ ID NO. 92, or variants thereof.
64. The multispecific antibody for use or method according to any one of clauses 50-59, wherein the multispecific antibody comprises CDR 1, 2 and 3 of MF6754 and CDRs 1, 2 and 3 of MF5561, or variants thereof.
65. The multispecific antibody for use or method according to clause 64, wherein the multispecific antibody comprises SEQ ID NO. 20 and SEQ ID NO. 100, or variants thereof.
66. The multispecific antibody for use or method according to any one of clauses 50-59, wherein the multispecific antibody comprises CDR 1, 2 and 3 of MF6785 and CDRs 1, 2 and 3 of MF5439, or variants thereof.
67. The multispecific antibody for use or method according to clause 66, wherein the multispecific antibody comprises SEQ ID NO. 31 and SEQ ID NO. 89, or variants thereof.
68. The multispecific antibody for use or method according to any one of clauses 50-59, wherein the multispecific antibody comprises CDR 1, 2 and 3 of MF6795 and CDRs 1, 2 and 3 of MF5442, or variants thereof.
69. The multispecific antibody for use or method according to clause 68, wherein the multispecific antibody comprises SEQ ID NO. 9 and SEQ ID NO. 92, or variants thereof.
70. The multispecific antibody for use or method according to any preceding clause, wherein the antibody comprises the CDR1, CDR2 and CDR3 sequences of the common light chain variable domain having an amino acid sequence as set forth in SEQ ID NO: 110, or the common light chain having an amino acid sequence as set forth in SEQ ID NO: 109, or variants thereof, wherein CDR nomenclature or numbering is according to IMGT.
71. The multispecific antibody for use or method according to any preceding clause, wherein the antibody comprises a common light chain variable domain having an amino acid sequence as set forth in SEQ ID NO: 110, or a common light chain having an amino acid sequence as set forth in SEQ ID NO: 109, or variants thereof.
72. The multispecific antibody for use or method according to any preceding clause, wherein the antibody comprises a heavy chain constant domain 1 (CH1) having an amino acid sequence as set forth in SEQ ID NO: 112, a heavy chain constant domain 2 (CH2) having an amino acid sequence as set forth in SEQ ID NO: 114, a heavy chain constant domain 3 (CH3) having an amino acid sequence as set forth in SEQ ID NO: 115, and a heavy chain constant domain 3 (CH3) having an amino acid sequence as set forth in SEQ ID NO: 116, or variants thereof.
73. The multispecific antibody or method according to any of clauses 50 to 72, wherein the PD-1 or PD-L1 inhibitor is a PD-1 inhibitor.
74. The multispecific antibody or method according to clause 73, wherein the PD-1 inhibitor is selected from Nivolumab, Pembrolizumab, Cemiplimab, Penpulimab, Retifanlimab, Sintilimab, Tislelizumab, Toripalimab and Dostarlimab.
75. The multispecific antibody or method according to clause 74, wherein the PD-1 inhibitor is Pembrolizumab.
76. The multispecific antibody or method according to any of clauses 50 to 72, wherein the PD-1 or PD-L1 inhibitor is a PD-L1 inhibitor.
77. The multispecific antibody or method according to clause 76, wherein the PD-L1 inhibitor is selected from Atezolizumab, Avelumab, and Durvalumab.
75 A kit of parts comprising a multispecific antibody as defined in any one of the preceding clauses and instructions for use of the multispecific antibody in combination with a PD-1 or PD-L1 inhibitor.
79. A kit of parts according to clause 78, further comprising a PD-1 or PD-L1 inhibitor.
80. The kit according to clause 78 or 79, wherein the PD-1 or PD-L1 inhibitor is a PD-1 inhibitor.
81. The kit according to clause 80, wherein the PD-1 inhibitor is selected from Nivolumab, Pembrolizumab, Cemiplimab and Dostarlimab.
82. The kit according to clause 81, wherein the PD-1 inhibitor is Pembrolizumab.
83. The kit according to clause 78 or 79, wherein the PD-1 or PD-L1 inhibitor is a PD-L1 inhibitor.
84. The kit according to clause 83, wherein the PD-L1 inhibitor is selected from Atezolizumab, Avelumab, and Durvalumab.
85. The kit according to any of clauses 78 to 84, further comprising instructions for use of the multispecific antibody at a dose, or flat dose, of between 10-75 mg, or between 25-75 mg or between 25-50 mg or between 25-40 mg or between 25-30 mg, or at 10 mg, or 25 mg or 30 mg or 40 mg or 50 mg or 60 mg or 70 mg or 75 mg.
86. The kit according to any of clauses 78 to 85, wherein the kit comprises instructions for use of the multispecific antibody in the treatment of any advanced or metastatic solid tumor.
87. The kit of parts according to clause 86, wherein the advanced or metastatic solid tumor is selected from: locally advanced or metastatic lung cancer, in particular non-small cell lung cancer (NSCLC); and locally advanced or metastatic melanoma.
88. A combination of a multispecific antibody that comprises an antigen binding site that binds an extracellular part of CD137 and an antigen binding site that binds an extracellular part of a second membrane protein and a PD-1 or PD-L1 inhibitor for use in the treatment of cancer in a subject in need thereof, wherein the multispecific antibody is as defined in any preceding clause and the inhibitor is as defined in any preceding clause.
89. A PD-1 or PD-L1 inhibitor, as defined in any preceding clause, for the treatment of cancer in a subject in need thereof, wherein the PD-L1 inhibitor is for simultaneous or sequential administration with a multispecific antibody that comprises an antigen binding site that binds an extracellular part of CD137 and an antigen binding site that binds an extracellular part of a second membrane protein.
90. A multispecific antibody that comprises an antigen binding site that binds an extracellular part of CD137 and an antigen binding site that binds an extracellular part of a second membrane protein for use in the treatment of cancer in a subject to which a PD-1 or PD-L1 inhibitor, as defined in any preceding clause, has been or will be administered.
91. A PD-1 or PD-L1 inhibitor, as defined in any preceding clause, for use in the treatment of cancer in a patient to which a multispecific antibody that comprises an antigen binding site that binds an extracellular part of CD37 and an antigen binding site that binds an extracellular part of a second membrane protein has been or will be administered.

### EXAMPLES

### Example 1: Multispecific Antibodies Binding PD-L1 and CD137

Multispecific antibodies comprising heavy chain variable regions as mentioned in Table 1 were obtained as described in WO2018/056821.

TABLE 1
SEQ ID NOs for heavy chain variable regions of multispecific
antibodies binding CD137 and PD-L1.

| CD137 binding | | PD-L1 binding | |
|---|---|---|---|
| domain ID | SEQ ID NOs | domain ID | SEQ ID NOs |
| MF6783 | SEQ ID NO: 1 | MF5554 | SEQ ID NO: 53 |
| MF6861 | SEQ ID NO: 5 | MF5576 | SEQ ID NO: 57 |
| MF6795 | SEQ ID NO: 9 | MF5578 | SEQ ID NO: 59 |
| MF6808 | SEQ ID NO: 13 | MF9375 | SEQ ID NO: 62 |
| MF6798 | SEQ ID NO: 17 | MF9376 | SEQ ID NO: 64 |
| MF6754 | SEQ ID NO: 20 | MF7702 | SEQ ID NO: 67 |
| MF6763 | SEQ ID NO: 24 | MF5359 | SEQ ID NO: 69 |
| MF6744 | SEQ ID NO: 28 | MF5377 | SEQ ID NO: 73 |
| MF6785 | SEQ ID NO: 31 | MF5382 | SEQ ID NO: 77 |
| MF6825 | SEQ ID NO: 35 | MF5424 | SEQ ID NO: 81 |
| MF6737 | SEQ ID NO: 39 | MF5426 | SEQ ID NO: 85 |
| MF6749 | SEQ ID NO: 43 | MF5439 | SEQ ID NO: 89 |
| MF6788 | SEQ ID NO: 46 | MF5442 | SEQ ID NO: 92 |
| MF6797 | SEQ ID NO: 49 | MF5553 | SEQ ID NO: 96 |
| | | MF5557 | SEQ ID NO: 97 |
| | | MF5561 | SEQ ID NO: 100 |
| | | MF5576 | SEQ ID NO: 103 |
| | | MF5594 | SEQ ID NO: 104 |
| | | MF5708 | SEQ ID NO: 107 |

TABLE 2
Any of the heavy chain variable regions binding CD137 can be combined with any
of the heavy chain variable regions binding PD-L1 in the multispecific antibodies
of the present disclosure. Variants of the variable heavy chain regions may also
be combined in the multispecific antibodies of the present disclosure.

| | MF6783 | MF6861 | MF6795 | MF6808 | MF6798 | MF6754 | MF6763 |
|---|---|---|---|---|---|---|---|
| MF5554 | X | X | X | X | X | X | X |
| MF5576 | X | X | X | X | X | X | X |
| MF5578 | X | X | X | X | X | X | X |
| MF9375 | X | X | X | X | X | X | X |
| MF9376 | X | X | X | X | X | X | X |
| MF7702 | X | X | X | X | X | X | X |
| MF5359 | X | X | X | X | X | X | X |
| MF5377 | X | X | X | X | X | X | X |
| MF5382 | X | X | X | X | X | X | X |
| MF5424 | X | X | X | X | X | X | X |
| MF5426 | X | X | X | X | X | X | X |
| MF5439 | X | X | X | X | X | X | X |
| MF5442 | X | X | X | X | X | X | X |
| MF5553 | X | X | X | X | X | X | X |
| MF5557 | X | X | X | X | X | X | X |
| MF5561 | X | X | X | X | X | X | X |
| MF5576 | X | X | X | X | X | X | X |
| MF5594 | X | X | X | X | X | X | X |
| MF5708 | X | X | X | X | X | X | X |
| | MF6744 | MF6785 | MF6825 | MF6737 | MF6749 | MF6788 | MF6797 |
| MF5554 | X | X | X | X | X | X | X |
| MF5576 | X | X | X | X | X | X | X |
| MF5578 | X | X | X | X | X | X | X |
| MF9375 | X | X | X | X | X | X | X |
| MF9376 | X | X | X | X | X | X | X |
| MF7702 | X | X | X | X | X | X | X |
| MF5359 | X | X | X | X | X | X | X |
| MF5377 | X | X | X | X | X | X | X |
| MF5382 | X | X | X | X | X | X | X |
| MF5424 | X | X | X | X | X | X | X |
| MF5426 | X | X | X | X | X | X | X |
| MF5439 | X | X | X | X | X | X | X |
| MF5442 | X | X | X | X | X | X | X |
| MF5553 | X | X | X | X | X | X | X |
| MF5557 | X | X | X | X | X | X | X |
| MF5561 | X | X | X | X | X | X | X |
| MF5576 | X | X | X | X | X | X | X |
| MF5594 | X | X | X | X | X | X | X |
| MF5708 | X | X | X | X | X | X | X |

### Example 2—a Phase 1, Open-Label, Dose-Escalation, Safety, Tolerability, and Preliminary Efficacy Study of MCLA-145, in Combination with Pembrolizumab, in Participants with Advanced or Metastatic Malignancies

### Treatment Groups and Duration:

An open-label, non-randomized, Phase 1 study was initiated to determine the safety, tolerability, and preliminary efficacy of a multispecific antibody targeting CD137 and PD-L1 in combination with a PD-1 or PD-L1 inhibitor, in adult participants with advanced or metastatic malignancies.

The following multispecific antibodies are suitable for use in this study and for use in the methods of the disclosure: MF6797×MF7702, MF6763×MF5442, MF6754×MF5561, MF6785×MF5439 and MF6785×MF5442, such as MF6797×MF7702. Each multispecific antibody comprises two VH's as specified by the MF numbers capable of binding CD137 and PD-L1 respectively, an Fc region with a KK/DE CH3 heterodimerization domain as indicated by SEQ ID NO: 115 and SEQ ID NO: 116, respectively, a CH2 domain as indicated by SEQ ID NO:114, a CH1 domain as indicated by SEQ ID NO: 112, and a common light chain as indicated by SEQ ID NO: 109. Especially a bispecific antibody comprising heavy chain sequences of MF6797×MF7702 is suitable for use in the study and methods of the disclosure.

A dose escalation study was performed to determine the MTD and/or RDE of a multispecific antibody in combination with a fixed dose of a PD-1 inhibitor in participants with advanced or recurrent/metastatic solid tumors. Participants received escalating doses of one of the exemplary multispecific antibodies indicated above, further referred to herein as the “study antibody” every 2 weeks until MTD or RDE was reached. The duration of each treatment cycle was 28 days with escalating doses. Participants also received a fixed dose of Pembrolizumab of 400 mg every six weeks.

### Overall Study Design

This is an open-label, nonrandomized, Phase 1 study to determine the safety, tolerability, and preliminary efficacy of MCLA-145 in adult participants with advanced or metastatic malignancies that will be conducted in two parts, dose escalation and dose expansion. MCLA-145 will be administered intravenously as a flat dose over 2 hours every 14 days in 28-day cycles in combination with Pembrolizumab, which will be administered as a flat dose every six weeks.

### Part 1: Dose Escalation

Part 1 is a dose escalation to determine the maximum tolerated dose (MTD) and/or recommended dose for expansion (RDE) of MCLA-145 administered every 14 days, in combination with Pembrolizumab, in participants with advanced or metastatic solid tumors.

Participants will be treated with MCLA-145 at a dose level of 10 mg in combination with fix dose of pembrolizumab. 3-6 evaluable patients will be included at each dose level; if no dose limiting toxicities (DLTs) are encountered in the first 28 day cycle, the dose of MCLA-145 will be escalated to the next level. A maximum of 6 evaluable patients can be included at each dose level until a MTD or RDE is reached, or a decision is taken by the sponsor based on the global safety of product.

The participants will follow initially the following schema:

| Cohort | MCLA 145* | Pembrolizumab |
|---|---|---|
| B1 | 10 mg every 14 days on a 28 days cycle | 400 mg every 6 |
| B2 | 15 mg every 14 days on a 28 days cycle | weeks |
| B3 | 25 mg every 14 days on a 28 days cycle | |

Further doses in combination may be explored, including at 30 mg, 35 mg, 40 mg, 45 mg, 50 mg, 55 mg, 60 mg, 65 mg, 70 mg, 75 mg, 100 mg, 125 mg and 150 mg. Pembrolizumab may also be provided at 200 mg, every 3 weeks.

### Part 2: Dose Confirmation/Safety Expansion

Part 2 is a dose expansion to confirm the dose of MCLA-145 in combination with Pembrolizumab, through further evaluation of safety, tolerability, pharmacokinetic, preliminary antitumor activity, and functional target engagement. Participants with advanced or metastatic tumors, will be enrolled as follows:

Adult men or women with advanced or metastatic solid tumors relapsed to PD1/PD-L1. Participants with NSCLC and melanoma with PD-L1>1%, Relapsed to PD1/PD-L1, that are considered to have benefit from combination with pembrolizumab by the site investigator

Any other tumor histology from Part 1, for which preliminary efficacy was observed with MCLA-145 or for which emerging data suggest responses, can be treated in Part 2, after Sponsor Medical Monitor agreement. The initial enrollment in expansion cohorts will be limited to 10 or 20 participants per indication.

Enrollment may be limited to PD-L1-positive tumors, defined as tumor expression ≥1%, determined at the site by any commercially available assay if supported by emerging data.

### Inclusion Criteria

Participants are eligible to be included in the study only if all of the following criteria apply. Participants with melanoma or NSCLC must meet all criteria listed under the All Participants section AND the applicable Tumor-Specific Criteria section below to confirm eligibility.

### All Participants

Ability to comprehend and willingness to sign a written ICF for the study.

At least 18 years of age at the time of signing the informed consent.

Willingness and ability to conform to and comply with all Protocol requirements, including, all scheduled visits, and Protocol procedures.

Life expectancy of ≥12 weeks.

ECOG performance status of 0 or 1.

Part 1: locally advanced or metastatic NSCLC or melanoma relapsed to PD-L1/PD-1 and positive (≥1%) for PD-L1 expression on tumor and/or tumor-associated immune cells (based on local testing after completion of most recent prior line of therapy)

Part 2: locally advanced or metastatic NSCLC or melanoma either relapsed to PD-L1/PD-1 or immunotherapy naïve and positive (≥1%) for PD-L1 expression on tumor and/or tumor-associated immune cells (based on local testing after completion of most recent prior line of therapy.

Measurable disease per RECIST v1.1 or Lugano Criteria.

Note: Tumor lesions located in a previously irradiated area or in an area subjected to other loco-regional therapy are considered measurable if clear progression has been demonstrated in the lesion.

Received prior standard therapy for advanced or recurrent/metastatic disease as applicable to tumor type, are intolerant to treatment, or refuse standard treatment.

Received a maximum of 4 prior systemic treatment regimens (inclusive of chemotherapy, immunotherapy, and targeted therapy regimens) for advanced or recurrent/metastatic disease unless approved by the medical monitor.

Received a maximum of 1 prior anti-PD-1 therapy containing immunotherapy regimen in the advanced/metastatic setting. Anti-PD-1 therapy in combination with chemotherapies, targeted therapies, or other immunotherapies is permitted.

Willingness to undergo pretreatment and on-treatment tumor biopsies to obtain tumor tissue.

Note: In participants where tumor tissue cannot safely be obtained, the participant may still enroll in the study upon discussion with the medical monitor.

Part 2 only: Participants with known PD-L1 status or a fresh tumor biopsy for evaluation of PD-L1 expression locally during screening.

Willingness to avoid pregnancy or fathering children based on the criteria below.

Men must agree to take appropriate precautions to avoid fathering children (with at least 99% certainty) from screening through 90 days after the last dose of MCLA-145 and must refrain from donating sperm during this period. Permitted methods that are at least 99% effective in preventing pregnancy should be communicated to the participants and their understanding confirmed.

Women of childbearing potential must have a negative serum pregnancy test at screening and before the first dose on Day 1 and must agree to take appropriate precautions to avoid pregnancy (with at least 99% certainty) from screening through 90 days after the last dose of MCLA-145. Permitted methods that are at least 99% effective in preventing pregnancy should be communicated to the participants and their understanding confirmed. Women of childbearing potential should refrain from donating oocytes from 30 days prior to the first dose of MCLA-145 and until 90 days after the last dose of MCLA-145.

Women of nonchildbearing potential (ie, surgically sterile with a hysterectomy and/or bilateral oophorectomy OR≥12 months of amenorrhea and at least 50 years of age) are eligible.

### for Combination Therapy

Patients that have received at least 2 months of monotherapy of MCLA 145 may join the study. These patients must not have reported liver toxicity Grade ≤2 while on monotherapy with MCLA 145.

### Tumor-Specific Criteria

Participants with Histolozically or Cytologically Confirmed Melanoma:

Histologically or cytologically confirmed cutaneous, acral, or mucosal melanoma.

Note: Participants with ocular or uveal melanoma are excluded.

Unresectable Stage III or Stage IV melanoma per current AJCC staging system that is not amenable to local therapy.

Documentation of V600-activating BRAF mutation status or consent to BRAF testing during the screening period if previously not tested.

If BRAF mutation-positive, must have received BRAF±MEK targeted therapy with documented progression.

Part 1 participants for combination therapy: relapsed (e.g., progressed after more than 6 months of treatment with at least a stable disease as best response) to PD-L1/PD-1 therapies

Part 2 participants for combination therapy:

relapsed (e.g., progressed after more than 6 months of treatment with at least a stable disease as best response) to PD-L1/PD-1 therapies naïve from PD-L1/PD-1 therapies

Part 2 participants only monotherapy: Must have documented progression on anti-PD-1 therapy as defined by meeting one of the criteria below:

Primary refractory: Must have received prior treatment of an anti-PD-1 therapy (alone or as part of a combination with anti-CTLA-4 therapy) in the advanced or metastatic setting for a minimum of 12 weeks and have PD as their best response to treatment. Participants who received prior treatment with anti-PD-1 therapy in the adjuvant setting for a minimum of 12 weeks and progressed are also allowed.

Secondary resistance: Must have received prior anti-PD-1 therapy (alone or as part of a combination) in the advanced or metastatic setting and achieved CR, PR, or SD but later had confirmed PD while receiving anti-PD-1 therapy (PD confirmed at least 4 weeks [no less than 28 days] later).

Participants with Histolozically or Cytologically Confirmed NSCLC:

Histologically or cytologically confirmed diagnosis of NSCLC (either nonsquamous or squamous).

Documentation of test results for mutations or gene rearrangements for EGFR, ALK, BRAF, and ROS1 if the tumor is of nonsquamous histology only (molecular testing is not currently part of diagnostic guidelines for predominantly squamous histology). If mutations or gene arrangements are present, participant must have progressed on or be intolerable to targeted therapy.

Unresectable advanced or metastatic NSCLC as per current AJCC staging system that is not amenable to local therapy.

Part 1 participants for combination therapy: relapsed (e.g., progressed after more than 6 months of treatment with at least a stable disease as best response) to PD-L1/PD-1 therapies

Part 2 participants for combination therapy:
relapsed (e.g., progressed after more than 6 months of treatment with at least a stable disease as best response) to PD-L1/PD-1 therapies
naïve from PD-L1/PD-1 therapies

Part 2 participants only: Must have documented progression on anti-PD-1 therapy as defined by meeting 1 of the criteria below:

Primary refractory: Must have received prior anti-PD-1 therapy (alone or as part of a combination) in the advanced or metastatic setting for a minimum of 12 weeks and have progressive disease (PD) as their best response to treatment.

Secondary resistance: Must have received prior anti-PD-1 therapy (alone or as part of a combination) in the advanced or metastatic setting and achieved CR, PR, or SD but later had confirmed PD while receiving anti-PD-1 therapy (PD confirmed at least 4 weeks [no less than 28 days] later).

Participants with Solid Tumor Indication of Confirmed MSI-H or dMMR Status:

MSI-H or dMMR solid tumor, as determined by a local laboratory using IHC or polymerase chain reaction methods and must also have tissue available for central confirmation of diagnosis.

Part 2 participants only who are considered primary refractory to anti-PD-1 therapy are defined as follows: received prior anti-PD-1 therapy (alone or as part of a combination) in the advanced or metastatic setting for a minimum of 12 weeks and have documented PD as their best response to treatment.

### Exclusion Criteria

Participants are excluded from the study if any of the following criteria apply:

The following B-cell neoplasms: Burkitt lymphoma, lymphoblastic leukemia/lymphoma, lymphoplasmacytic lymphoma, chronic lymphocytic leukemia.

Prior therapy containing a 4-1BB agonist or prior therapy with CAR T-cell therapy.

Treatment with anticancer medications or investigational drugs within the following intervals before the first dose of MCLA-145:

At least 14 days for chemotherapy (6 weeks for mitomycin C and nitrosoureas), targeted small molecule therapy, or radiation therapy.

Note: Participant must not have had radiation pneumonitis as a result of treatment. A 1-week washout period is permitted for palliative radiation to non-CNS disease with sponsor approval.

At least 28 days for a prior mAb used for anticancer therapy.

Note: Participants receiving bisphosphonates and/or denosumab are eligible for enrollment.

For other agents with long half-lives (e.g., >5 days), enrollment before the fifth half-life requires medical monitor approval.

At least 10 weeks for radioimmunotherapy.

Must not have had an allogeneic SCT within the last 6 months or an autologous SCT within the last 3 months.

Has not recovered to ≤Grade 1 or baseline from toxic effects of prior therapy (including prior immunotherapy) and/or complications from prior surgical intervention before starting MCLA-145.

Note: Alopecia and stable neuropathy (≤Grade 2) are allowed.

No prior anti-PD-1 therapy (±anti-CTLA-4 therapy)-associated liver toxicity >Grade 1.

Only for patients treated in combination: baseline transaminases and bilirubin out of normal limits

Prior≥Grade 3 immune-mediated adverse events with anti-PD-1 therapy.

Note: The following Grade 3 or higher adverse events are permitted: Grade 3 rashes that resolved with topical therapy; immune-mediated adrenal insufficiency, Type 1 diabetes mellitus, or other endocrine abnormalities due to previous immunotherapy that are medically stable and adequately managed on a stable dose of replacement therapy; and asymptomatic amylase or lipase elevations that did not require treatment interruption.

History of any grade immune-mediated ocular adverse events.

Participants with laboratory values at screening defined following table.

### Exclusionary Laboratory Values

| | |
|---|---|
| Laboratory Parameter | Exclusion Criterion |

Hematology

| | | |
|---|---|---|
| a | Platelets | <100 × 10⁹/L |
| b | Hemoglobin | <9 g/dL or 5.6 mmol/L |
| c | ANC | <1.5 × 10⁹/L |

Hepatic

| | | |
|---|---|---|
| d | ALT | ≥1.5 × ULN |
| e | AST | ≥1.5 × ULN |
| f | Total bilirubin | ≥1.2 × ULN unless conjugated bilirubin |
| | | is ≤ULN (conjugated bilirubin only needs to be tested if |
| | | total bilirubin exceeds the ULN). If there is no |
| | | institutional ULN, then conjugated bilirubin must |
| | | be <40% of total bilirubin. |
| g | Alkaline phosphatase | ≥2.5 × ULN |
| | | Participants with bone metastases and no |
| | | hepatic parenchymal metastases on screening |
| | | radiographic examinations may enroll if alkaline |
| | | phosphatase is ≤5.0 × ULN. |
| | | Participants with bone metastases and hepatic |
| | | parenchymal metastases on screening radiographic |
| | | examinations may enroll if alkaline phosphatase |
| | | is ≤5.0 × ULN only with medical monitor approval. |

Renal

| | | |
|---|---|---|
| h | Clearance of creatinine | creatinine clearance <30 mL/min |

Coagulation

| | | |
|---|---|---|
| i | International | >1.5 × ULN |
| | normalized ratio or | |
| | prothrombin time | |
| j | Activated partial | >1.5 × ULN |
| | thromboplastin time | |

Other

| | | |
|---|---|---|
| k | Albumin | <3 g/dL |

Clinically significant cardiac disease, including known history of left ventricular ejection fraction of <5000, unstable angina, acute myocardial infarction within 6 months of Cycle 1 Day 1, New York Heart Association Class III or IV congestive heart failure, or arrhythmia requiring therapy.

Note: A participant with an arrhythmia may enroll if the participant is on antiarrhythmic medication and is in sinus rhythm on the screening ECG.

History or presence of an ECG that, in the investigator's opinion, is clinically meaningful.

Immune-related toxicity during prior checkpoint inhibitor therapy for which permanent discontinuation of therapy is recommended (per product label or consensus guidelines), OR any immune-related toxicity requiring intensive or prolonged immunosuppression to manage (with the exception of endocrinopathy that is well controlled on replacement hormones).

History of idiopathic pulmonary fibrosis, interstitial lung disease, organizing pneumonia (e.g., bronchiolitis obliterans), drug-induced pneumonitis or idiopathic pneumonitis, or evidence of active pneumonitis on screening chest CT scan. Participants with a history of uncontrolled asthma or chronic obstructive pulmonary disease are excluded.

Note: History of radiation pneumonitis in the radiation field (fibrosis) is permitted.

Known active CNS metastases/lymphoma and/or carcinomatous meningitis.

Note: Participants with previously-treated brain metastases may participate provided they are 1) radiologically stable (ie, without evidence of progression for at least 28 days by repeat imaging prior to the first dose of MCLA-145 [repeat imaging may be performed during the screening period with medical monitor approval]), 2) without requirement of steroid treatment for at least 14 days prior to first dose of MCLA-145, and 3) clinically stable, with any neurological signs or symptoms having returned to baseline. This exception does not include carcinomatous meningitis, which is excluded regardless of clinical stability.

Note: Participants with evidence of cerebral edema or those with <28 days since radiation therapy to the CNS will be excluded from study.

Participants who have active or inactive autoimmune disease or syndrome (eg, rheumatoid arthritis, moderate or severe psoriasis, multiple sclerosis, inflammatory bowel disease) that has required systemic treatment in the past 2 years or who are receiving systemic therapy for an autoimmune or inflammatory disease (ie, with use of disease modifying agents, corticosteroids, or immunosuppressive drugs).

Note: Participants with vitiligo, resolved childhood asthma/atopy, hypothyroidism stable on hormone replacement, controlled asthma, Type I diabetes, Graves' disease, or Hashimoto's disease, or with medical monitor approval, will be eligible if they meet all other eligibility criteria.

Use of systemic corticosteroids (≥10 mg/day prednisone or equivalent) within 7 days before the first dose of MCLA-145.

Note: The use of inhaled or topical corticosteroids or systemic corticosteroids for imaging procedures is permitted.

Note: The use of physiologic corticosteroid replacement therapy may be approved after consultation with the medical monitor.

Receipt of a live vaccine within 30 days of the first dose of MCLA-145 or pembrolizumab.

Note: Examples of live vaccines include, but are not limited to, the following: measles, mumps, rubella, chicken pox, yellow fever, rabies, Bacillus Calmette-Guérin, and typhoid vaccine. If COVID-19 live vaccines are available, consultation with the medical monitor is required prior to administration. Seasonal influenza vaccines for injection are generally killed virus vaccines and are allowed; however, intranasal influenza vaccines are live attenuated vaccines and are not allowed.

Active infection requiring systemic therapy.

History of solid organ or allogeneic stem cell transplant.

Active HBV or HCV infection that requires treatment. Hepatitis B virus DNA and HCV RNA must be undetectable. Participants who have cleared a prior HBV infection (defined as hepatitis B surface antigen negative, hepatitis B surface antibody positive, hepatitis B core antibody positive) are eligible for the study.

Note: For participants with a cleared prior HBV infection, HBV prophylaxis should be considered per the investigator's discretion. Monitor for HBV reactivation every 3 cycles by performing HBV viral load and hepatitis B surface antigen serology testing. Additional viral serologic testing may be performed at the investigator's discretion.

Note: Participants with no prior history of HBV infection who have been vaccinated against HBV and who have a positive antibody against hepatitis B surface antigen as the only evidence of prior exposure may participate in the study.

Note: Hepatitis C antibody-positive participants who received and completed treatment for hepatitis C that was intended to eradicate the virus may participate if HCV RNA levels are undetectable.

Known history of HIV (HIV 1/2 antibodies). No HIV testing is required unless mandated by local health authority or regulations.

Known hypersensitivity or severe reaction to any component of MCLA-145 or Pembrolizumab or formulation components.

Is pregnant or breastfeeding or expecting to conceive or father children within the projected duration of the study, starting with the screening visit through 90 days after the last dose of MCLA-145.

Any condition that would, in the investigator's judgment, interfere with full participation in the study, including administration of MCLA-145 or pembrolizumab; pose a significant risk to the participant; or interfere with interpretation of study data.

### Example 3

An 80 year old male patient diagnosed with Merkel Cell Carcinoma, having a PD-L1 score of 1-10%. The patient was pretreated with radiotherapy, surgery and systemic therapy. Treatment with avelumab resulted in progressive disease. Carboplatin and etoposide treatment resulted in a partial response.

Subsequently, the patient was administered a dose of 25 mg every three weeks (25 mg, q3w) of a multispecific antibody having two VH's as specified by MF numbers MF6797 and MF7702, an Fc region with a KK/DE CH3 heterodimerization domain as indicated by SEQ ID NO:115 and SEQ ID NO:116, respectively, a CH2 domain as indicated by SEQ ID NO:114, a CH1 domain as indicated by SEQ ID NO: 112, and a common light chain as indicated by SEQ ID NO: 109 and a dose of 200 mg pembrolizumab every three weeks (200 mg, q3w). Site of disease were the cutaneous nodes of the foot. Tumor assessment involved the sum of target lesion diameters. After the initial dose, the patient developed a clinically significant reduction of lesion diameter.

### Example 4

A bispecific antibody having two VH's as specified by MF numbers MF6797 and MF7702, an Fc region with a KK/DE CH3 heterodimerization domain as indicated by SEQ ID NO:115 and SEQ ID NO:116, respectively, a CH2 domain as indicated by SEQ ID NO:114, a CH1 domain as indicated by SEQ ID NO: 112, and a common light chain as indicated by SEQ ID NO: 109 is administered to a subject having a cancer using a dose of 40 mg every three weeks (40 mg, q3w) or every two weeks (40 mg, q2w) along with administration of 200 mg pembrolizumab every three weeks (200 mg, q3w) in accordance with Example 2, after which tumor assessment takes place involving the sum of target lesion diameters. After the initial dose, the patient is allowed to developed a clinically significant reduction of lesion diameter.

### SEQUENCES

SEQ ID NO: 1: Heavy chain variable region
QVQLVQSGSELKKPGASVKVSCKASGYTFTNFAMNWVRRAPGQGLEWMGWINTNT
GNPTYAQGFTGRFVFSLDTSVNTAYLQISSLKAEDTAVYYCARDWGVIGGHYMDV
WGKGTTVTVSS
SEQ ID NO: 2: HCDR1 according to Kabat from SEQ ID NO: 1
NFAMN
SEQ ID NO: 3: HCDR2 according to Kabat from SEQ ID NO: 1
WINTNTGNPTYAQGFTG
SEQ ID NO: 4: HCDR3 according to Kabat from SEQ ID NO: 1
DWGVIGGHYMDV
SEQ ID NO: 5 Heavy chain variable region
QVQLVQSGAEVKKPGASVKVSCKASGYTFTSYGISWVRQAPGQGLEWMGWISAYN
GNTNYAQKLQGRVTMTTDTSTSTAYMELRSLRSDDTAVYYCARDSDGYGPKAFDY
WGQGTLVTVSS
SEQ ID NO: 6: HCDR1 according to Kabat
SYGIS
SEQ ID NO: 7: HCDR2 according to Kabat
WISAYNGNTNYAQKLQG
SEQ ID NO: 8: HCDR3 according to Kabat
DSDGYGPKAFDY
SEQ ID NO: 9 Heavy chain variable region
EVQLVQSGAEVKKPGESLKISCKGSGYSFTSYWIGWVRQMPGKGLEWMGIIYPDD
SDTRYSPSFQGQVTISADKSSSTAYLQWSSLKASDTAMYYCASFYTGIVGATGAF
DVWGQGTTVTVSS
SEQ ID NO: 10: HCDR1 according to Kabat
SYWIG
SEQ ID NO: 11: HCDR2 according to Kabat
IIYPDDSDTRYSPSFQG
SEQ ID NO: 12: HCDR3 according to Kabat
FYTGIVGATGAFDV
SEQ ID NO: 13 Heavy chain variable region
QVQLVQSGAEVKKPGSSVKVSCKASGGTFSSDAISWVRQAPGQGLEWMGGMIPIL
GTANYAQKFQGRVTITADRSTSTAYMELSSLRSEDTAVYYCVRGATYYYGSGTYY
SINWFDPWGQGTLVTVSS
SEQ ID NO: 14: HCDR1 according to Kabat
SDAIS
SEQ ID NO: 15: HCDR2 according to Kabat
GMIPILGTANYAQKFQG
SEQ ID NO: 16: HCDR3 according to Kabat
GATYYYGSGTYYSINWFDP
SEQ ID NO: 17 Heavy chain variable region
QVQLVQSGSELKKPGASVKVSCRASGYTFTNFAMTWVRQAPGQGPEYMGWINTNT
GNPTYAQGFTGRFVFSLDTSVNTAYLQISSLKAEDTAVYYCARDWASVMVRGDLD
YWGQGTLVTVSS
SEQ ID NO: 18: HCDR1 according to Kabat
NFAMT
SEQ ID NO: 19: HCDR3 according to Kabat
DWASVMVRGDLDY
SEQ ID NO: 20 Heavy chain variable region
QVQLVQSGAEVKKPGASVKVSCKVSGYTLSELSIHWVRQAPGKGVEWMGGFYPED
VEPIYARKFQGRVTMTEDTSTDTAYMELNSLRSEDTAVYYCAAEGFDNYGSGIRG
NWFDPWGQGTLVTVSS
SEQ ID NO: 21: HCDR1 according to Kabat
ELSIH
SEQ ID NO: 22: HCDR2 according to Kabat
GFYPEDVEPIYARKFQG
SEQ ID NO: 23: HCDR3 according to Kabat
EGFDNYGSGIRGNWFDP
SEQ ID NO: 24 Heavy chain variable region
EVQLVQSGAEVKKPGASVKVSCKVSGYTLTELSMHWVRQSPGKGLEWMGSFYPED
GETIYAQKFQGRITMTEDTSADTAYMELSSLRSEDTAVYYCATEGVGVIRGNWFD
PWGQGTLVTVSS
SEQ ID NO: 25: HCDR1 according to Kabat
ELSMH
SEQ ID NO: 26: HCDR2 according to Kabat
SFYPEDGETIYAQKFQG
SEQ ID NO: 27: HCDR3 according to Kabat
EGVGVIRGNWFDP
SEQ ID NO: 28 Heavy chain variable region
EVQLVQSGAEVKKPGESLKISCKGSGYSFTSYWIGWVRQMPGKGLEWMGIIFPDD
SDTRYSPSFQGQVTISADKSISTAYLQWSSLKPSDTAMYYCVRLGGYSGYAEDFV
DFWGQGTLVTVSS
SEQ ID NO: 29: HCDR2 according to Kabat
IIFPDDSDTRYSPSFQG
SEQ ID NO: 30: HCDR3 according to Kabat
LGGYSGYAEDFVDF
SEQ ID NO: 31 Heavy chain variable region
EVQLVQSGAEVKKPGASVKVSCKVSGYTLTKLSMHWVRQAPGKGLEWMGGFEPED
GETINAQKFQGRVTMTEDTSTDTAYMELSSLRSEDTAVYYCATDLRLGASYYYSY
MDVWGRGTMVTVSS
SEQ ID NO: 32: HCDR1 according to Kabat
KLSMH
SEQ ID NO: 33: HCDR2 according to Kabat
GFEPEDGETINAQKFQG
SEQ ID NO: 34: HCDR3 according to Kabat
DLRLGASYYYSYMDV
SEQ ID NO: 35 Heavy chain variable region
QITLKESGPTLVKPTQTLTLSCTFSGFSLSTSGMSVGWIRQPPGKALEWLALIYW
NDDKYFSPSLKSRLTITKDTSKNQVVLTLTNMDPVDTATYYCAHTLWGSDDVFDV
WGQGTMVTVSS
SEQ ID NO: 36: HCDR1 according to Kabat
TSGMSVG
SEQ ID NO: 37: HCDR2 according to Kabat
LIYWNDDKYFSPSLKS
SEQ ID NO: 38: HCDR3 according to Kabat
TLWGSDDVFDV
SEQ ID NO: 39 Heavy chain variable region
EVQLVQSGAEVKKPGESLKISCKVSGYSFTNYWIGWVRQMPGKGLEWMGIIYPGD
SDTRYSPSFQGQVTISADKSISTAYLQWHTLKASDTAMYYCARHQGYSFSGSHID
DYWGQGTLVTVSS
SEQ ID NO: 40: HCDR1 according to Kabat
NYWIG
SEQ ID NO: 41: HCDR2 according to Kabat
IIYPGDSDTRYSPSFQG
SEQ ID NO: 42: HCDR3 according to Kabat
HQGYSFSGSHIDDY
SEQ ID NO: 43 Heavy chain variable region
EVQLVQSGAEVRKPGESLKISCKGSGYSFTTYWIGWVRQMPGKGLEWMGIIYPGD
SDTRYSPSFQGQVTISADKSISTVYLQWSSLKASDTAMYYCARHAGFIITSQNID
DYWGQGTLVTVSS
SEQ ID NO: 44: HCDR1 according to Kabat
TYWIG
SEQ ID NO: 41: HCDR2 according to Kabat
IIYPGDSDTRYSPSFQG
SEQ ID NO: 45: HCDR3 according to Kabat
HAGFIITSQNIDDY
SEQ ID NO: 46 Heavy chain variable region
EVQLVQSGSELKKPGASVKVSCKASGYTFTNFAMNWVRQAPGQGLEWMGWINTNT
GNPTYAQDFTGRFVFSLDTSGNTAYLQISSLKAEDTAVYYCARDWGLVAIGYFDY
WGQGTLVTVSS
SEQ ID NO: 47: HCDR2 according to Kabat
WINTNTGNPTYAQDFTG
SEQ ID NO: 48: HCDR3 according to Kabat
DWGLVAIGYFDY
SEQ ID NO: 49 Heavy chain variable region
QITLKESGPTLVKPTQTLTLTCTFSGFSLSTTGVGVNWIRQPPGEALEWLALIYW
NDDTYYSPSLKSRLTITKDTSKNQVVLTMTNMDPVDTATYYCAHEGIIGFLGGNW
FDPWGQGTLVTVSS
SEQ ID NO: 50: HCDR1 according to Kabat
TTGVGVN
SEQ ID NO: 51: HCDR2 according to Kabat
LIYWNDDTYYSPSLKS
SEQ ID NO: 52: HCDR3 according to Kabat
EGIIGFLGGNWFDP
SEQ ID NO: 53 Heavy chain variable region
QVQLVQSGSELKKPGASVKVSCKASGYTFTSHAMNWVRQAPGQGLEWMGWINPNT
GNPTYAQGFTGRFVFSLDTSVSTAYLQISSLKAEDTAVYYCARDRKYVTNWVFAE
DFQHWGQGTLVTVSS
SEQ ID NO: 54: HCDR1 according to Kabat
SHAMN
SEQ ID NO: 55: HCDR2 according to Kabat
WINPNTGNPTYAQGFTG
SEQ ID NO: 56: HCDR3 according to Kabat
DRKYVTNWVFAEDFQH
SEQ ID NO: 57 Heavy chain variable region
QVQLVQSGSELKKPGASVKVSCKASGYTFTSHAMNWVRQAPGQGLEWMGWINPNT
GNPTYAQGFTGRFVFSLDTSVSTAYLQISSLKAEDTAVYYCAIDRGYMSNWVFAE
YFPHWGQGTLVTVSS
SEQ ID NO: 58: HCDR3 according to Kabat
DRGYMSNWVFAEYFPH
SEQ ID NO: 59 Heavy chain variable region
QVQLVQSGSELKKPGASVKVSCKASGYTFTSYAMNWVRQAPGQGLEWMGWINTNT
GNPTYAQGFTGRFVFSLDTSVSTAYLQISSLKAEDTAVYYCATDRGYISSWVFAE
DFQHWGQGTLVTVSS
SEQ ID NO: 60: HCDR1 according to Kabat
SYAMN
SEQ ID NO: 61: HCDR3 according to Kabat
DRGYISSWVFAEDFQH
SEQ ID NO: 62 Heavy chain variable region
QVQLVQSGSELKKPGASVKVSCTASGYTFTSYAMNWVRQAPGQRLEWMACVNPNT
GSPTYAQGSTGRFVVSLDTSVSTAYLQISSLKAEDTAVYYCARDRKYVTNWVFAE
DFQHWGHGTLVTVSS
SEQ ID NO: 63: HCDR2 according to Kabat
CVNPNTGSPTYAQGSTG
SEQ ID NO: 64 Heavy chain variable region
QVQLVQSGSELKKPGASVKVSCKASGYTFTNYAMNWVRQAPGQGLEWMGWMNPNT
GNPTYAQGSTGRFVVSLDTSVSTAYLQISSLKAEDTAVYYCARDRKYVTNWVFAE
DFQHWGRGTLVTVSS
SEQ ID NO: 65: HCDR1 according to Kabat
NYAMN
SEQ ID NO: 66: HCDR2 according to Kabat
WMNPNTGNPTYAQGSTG
SEQ ID NO: 67 Heavy chain variable region
QVQLVQSGSELKKPGASVKVSCKASGYTFTNYAINWVRQAPGQGLEWMGWINPNT
GNPTYAQGFTGRFVFSLDTSVSTAYLQISSLKAEDTAVYYCARDRKYVTNWVFAE
DFQHWGRGTLVTVSS
SEQ ID NO: 68: HCDR1 according to Kabat
NYAIN
SEQ ID NO: 69 Heavy chain variable region
EVQLVQSGAEVKKPGSSVKVSCKASGDTFNTYSITWVRQAPGQGLEWMGSIVPIF
GTINNAQKFQGRVTITADKSANTAYMELSSLRSEDTAVYYCARDNTMVRGVDYYY
MDVWGKGTMVTVSS
SEQ ID NO: 70: HCDR1 according to Kabat
TYSIT
SEQ ID NO: 71: HCDR2 according to Kabat
SIVPIFGTINNAQKFQG
SEQ ID NO: 72: HCDR3 according to Kabat
DNTMVRGVDYYYMDV
SEQ ID NO: 73 Heavy chain variable region
EVQLVQSGAEVKKPGSSVKVSCKASGGIFSTYAISWVRQAPGQGLEWMGGIIPIF
DTPNYAQKFQGRVTITADKSTSTAYMDLSSLRSEDTAVYYCAKNVRGYSAYDLDY
WGQGTLVTVSS
SEQ ID NO: 74: HCDR1 according to Kabat
TYAIS
SEQ ID NO: 75: HCDR2 according to Kabat
GIIPIFDTPNYAQKFQG
SEQ ID NO: 76: HCDR3 according to Kabat
NVRGYSAYDLDY
SEQ ID NO: 77 Heavy chain variable region
EVQLVQSGAEVKNPGSSVKVSCKATGGTFNTYGTNWVRQAPGQGLEWMGGIIPIF
GTANYAQKFQGRVTITADKSTTTAYMEVSSLRSEDTAVYYCARGGADMGTLDYWG
QGTLVTVSS
SEQ ID NO: 78: HCDR1 according to Kabat
TYGTN
SEQ ID NO: 79: HCDR2 according to Kabat
GIIPIFGTANYAQKFQG
SEQ ID NO: 80: HCDR3 according to Kabat
GGADMGTLDY
SEQ ID NO: 81 Heavy chain variable region
EVQLVQSGAEVMRPGSSVKVSCKASGGIFNTYTIIWVRQAPGQGLEWMGGIIPIF
DTPNFAQKFQGRLTITADKSTNTAYMELTSLRSEDTAVYYCAREGCNHGVCYPYW
GQGTLVTVSS
SEQ ID NO: 82: HCDR1 according to Kabat
TYTII
SEQ ID NO: 83: HCDR2 according to Kabat
GIIPIFDTPNFAQKFQG
SEQ ID NO: 84: HCDR3 according to Kabat
EGCNHGVCYPY
SEQ ID NO: 85 Heavy chain variable region
QVQLVQSGAEVKKPGSSVKVSCKASGDTFRSYGITWVRQAPGQGLEWMGGIIPIF
GTTNYAQKFQGRVTITADKSTSTVYMELSSLRSEDTAVYYCARRRGYSNPHWLDP
WGQGTLVTVSS
SEQ ID NO: 86: HCDR1 according to Kabat
SYGIT
SEQ ID NO: 87: HCDR2 according to Kabat
GIIPIFGTTNYAQKFQG
SEQ ID NO: 88: HCDR3 according to Kabat
RRGYSNPHWLDP
SEQ ID NO: 89 Heavy chain variable region
QVQLVQSGAEVKKPGSSVKVSCKASGGTFSTYGILWVRQAPGQGLEWMGGIIPIF
GTANYAQKFQGRVTITADISTSTAYMELSSLRSEDTAVYYCARGGGNYYEFVYWG
QGTLVTVSS
SEQ ID NO: 90 HCDR1 according to Kabat
TYGIL
SEQ ID NO: 91: HCDR3 according to Kabat
GGGNYYEFVY
SEQ ID NO: 92 Heavy chain variable region
EVQLVQSGAEVKKPGSSVRVSCKASGGTFNTYAINWVRQAPGQGLEWVGRIIPIF
DTANYAQKFQGRVTISADKSTTTAYMELSSLRSEDTAVFYCAKDETGYSSSNFQH
WGQGTLVTVSS
SEQ ID NO: 93 HCDR1 according to Kabat
TYAIN
SEQ ID NO: 94 HCDR2 according to Kabat
RIIPIFDTANYAQKFQG
SEQ ID NO: 95 HCDR3 according to Kabat
DETGYSSSNFQH
SEQ ID NO: 96 Heavy chain variable region
QVQLVQSGSELKKPGASVKVSCKASGYTFTNYAINWVRQAPGQGLEWMGWINPNT
GNPTYAQGFTGRFVFSLDTSVSTAYLQISSLKAEDTAVYYCARDRKYVTNWVFAE
DFQHWGQGTLVTVSS
SEQ ID NO: 97 Heavy chain variable region
QVQLVQSGAEVKRPGSSVKVSCKASGGTFNTYSITWVRQAPGQGLEWMGGIIPVF
GTSKYAQKFQDRVTITADKSTNTAYMELSSLRSEDTAVYYCARDPSFSSSSGWFD
PWGQGTLVTVSS
SEQ ID NO: 98 HCDR2 according to Kabat
GIIPVFGTSKYAQKFQD
SEQ ID NO: 99: HCDR3 according to Kabat
DPSFSSSSGWFDP
SEQ ID NO: 100 Heavy chain variable region
QVQLVQSGAEVKKPGSSVKVSCKASGGTFNTYAINWVRQAPGQGLEWMGGIIPIF
DTANYAQRFQGRVTITADKSTSTAYMELSSLRSEDTAVYFCAKDQTGYSSTLFDY
WGQGTLVTVSS
SEQ ID NO: 101 HCDR2 according to Kabat
GIIPIFDTANYAQRFQG
SEQ ID NO: 102 HCDR3 according to Kabat
DQTGYSSTLFDY
SEQ ID NO: 103 Heavy chain variable region
QVQLVQSGSELKKPGASVKVSCKASGYTFTSHAMNWVRQAPGQGLEWMGWINPNT
GNPTYAQGFTGRFVFSLDTSVSTAYLQISSLKAEDTAVYYCAIDRGYMSNWVFAE
YFPHWGQGTLVTVSS
SEQ ID NO: 104 Heavy chain variable region
EVQLVQSGAEVKKPGSSVKVSCKASGGTFSTYAISWVRQAPGQGLEWMGWIIPIF
DTGNYAQKIQGRVTITADKSTSTAYMELTSLRSEDTAVYYCARHDYTNTVDAFDI
WGQGTMVTVSS
SEQ ID NO: 105 HCDR2 according to Kabat
WIIPIFDTGNYAQKIQG
SEQ ID NO: 106 HCDR3 according to Kabat
HDYTNTVDAFDI
SEQ ID NO: 107 Heavy chain variable region
QVQLVQSGAEVKKPGSSVKVSCKASGDTFRSYGITWVRQAPGQGLEWMGGIIPVF
GTTNYAQKFQGRVTITADKSTSTVFMELNSLRSEDTAVYYCARRRGYSNPHWLDP
WGQGTLVTVSS
SEQ ID NO: 108 HCDR2 according to Kabat
GIIPVFGTTNYAQKFQG
SEQ ID NO: 109 Amino acid sequence of human common light
chain IGKV1-39/jk1
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPTFGQGTKVEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 110 Amino acid sequence of common light
chain variable domain
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPTFGQGTKVEIK
SEQ ID NO: 111 Amino acid sequence of common light
chain constant domain
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
SEQ ID NO: 112 Amino acid sequence of CH1
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRV
SEQ ID NO: 113 Amino acid sequence of the hinge
EPKSCDKTHTCPPCP
SEQ ID NO: 114 Amino acid sequence of CH2
APELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAK
SEQ ID NO: 115 Amino acid sequence of CH3 with KK
mutations
GQPREPQVYTKPPSREEMTKNQVSLKCLVKGFYPSDIAVEWESNGQPENNYKTTP
PVLDSDGSFFLYSKLTVDKSRWQQGNVFSCSVMHEALHNHYTQKSLSLSPGK
SEQ ID NO: 116 Amino acid sequence of CH3 with DE
mutations
GQPREPQVYTDPPSREEMTKNQVSLTCEVKGFYPSDIAVEWESNGQPENNYKTTP
PVLDSDGSFFLYSKLTVDKSRWQQGNVFSCSVMHEALHNHYTQKSLSLSPGK
SEQ ID NO: 117 Amino acid sequence of CD137 according
to HGNC: 11924
MGNSCYNIVATLLLVLNFERTRSLQDPCSNCPAGTFCDNNRNQICSPCPPNSFSS
AGGQRTCDICRQCKGVFRTRKECSSTSNAECDCTPGFHCLGAGCSMCEQDCKQGQ
ELTKKGCKDCCFGTFNDQKRGICRPWTNCSLDGKSVLVNGTKERDVVCGPSPADL
SPGASSVTPPAPAREPGHSPQIISFFLALTSTALLFLLFFLTLRFSVVKRGRKKL
LYIFKQPFMRPVQTTQEEDGCSCRFPEEEEGGCEL
SEQ ID NO 118 Amino acid sequence of Pembrolizumab
heavy chain
QVQLVQSGVEVKKPGASVKVSCKASGYTFTNYYMYWVRQAPGQGLEWMGGINPSN
GGTNFNEKFKNRVTLTTDSSTTTAYMELKSLQFDDTAVYYCARRDYRFDMGFDYW
GQGTTVTVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALHNHYTQKS
LSLSLGK
SEQ ID NO 119 Amino acid sequence of Pembrolizumab
light chain
EIVLTQSPATLSLSPGERATLSCRASKGVSTSGYSYLHWYQQKPGQAPRLLIYLA
SYLESGVPARFSGSGSGTDFTLTISSLEPEDFAVYYCQHSRDLPLTFGGGTKVEI
KRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC

1. (canceled)

2. A method of treating cancer in a subject in need thereof, the method comprising administering a multispecific antibody that comprises an antigen binding site that binds an extracellular part of CD137, comprising a heavy chain variable domain comprising CDRs 1, 2, and 3 of SEQ ID NO: 49 and a light chain variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121); and an antigen binding site that binds an extracellular part of PD-L1 comprising a heavy chain variable domain comprising CDRs 1, 2, and 3 of SEQ ID NO: 67 and a light chain variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121), and a Pembrolizumab.

3. The method according to claim 2, wherein the multispecific antibody is administered simultaneously, sequentially, or separately with the Pembrolizumab.

4. The method according to claim 2, wherein the cancer is an advanced or metastatic solid tumor, such as selected from locally advanced or metastatic lung cancer and locally advanced or metastatic melanoma, such as cutaneous, acral or mucosal melanoma.

5. The method according claim 2, wherein the cancer is Merkel Cell Carcinoma (MCC) in a human.

6-7. (canceled)

8. The method according to claim 2, wherein the cancer is relapsed to a PD-1/PD-L1 therapy and/or is positive for PD-L1 expression.

9-11. (canceled)

12. The method according to claim 2, wherein the multispecific antibody is administered in a dose of between 10 mg-1200 mg, 15-1200 mg or 25-1200 mg, or is administered in a dose of 10 mg, 15 mg, 20 mg, 25 mg, 30 mg, 35 mg, 40 mg, 45 mg, 50 mg, 60 mg or 75 mg.

13. (canceled)

14. The method according to claim 2, wherein the PD-1 inhibitor is Pembrolizumab and is administered in a dose of 300-500 mg or 100-300 mg.

15. (canceled)

16. The method according to claim 2, wherein the multispecific antibody is administered every 14 days or every 21 days.

17-18. (canceled)

19. The method according to claim 2, wherein the multispecific antibody is administered intravenously and administered once every two weeks or once every three weeks.

20-32. (canceled)

33. The method according to claim 2, wherein said multispecific antibody is a full length antibody or an IgG1 molecule without Fc effector function.

34-46. (canceled)

47. The method according to any one of the preceding claims, wherein the multispecific antibody comprises SEQ ID NO. 49 having 0, 1, 2, 3, 4 or 5 amino acid insertions, deletions, substitutions or a combination thereof and SEQ ID NO. 67 having 0, 1, 2, 3, 4 or 5 amino acid insertions, deletions, substitutions or a combination thereof, wherein the amino acid insertion(s), deletion(s), substitution(s) or a combination thereof are not in the amino acid sequence of the CDR regions.

48. A method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52;
and a variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121):
and
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56;
and a variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121):
wherein the multispecific antibody is administered simultaneously, sequentially, or separately with a Pembrolizumab.

49. (canceled)

50. A method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52;
and a variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121):
a variable domain that comprises a CDR1 having an amino acid sequence as set forth in SEQ ID NO: 40, a CDR2 having an amino acid sequence as set forth in SEQ ID NO: 41 and a CDR3 having an amino acid
and
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56;
and a variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121):
wherein the multispecific antibody is administered simultaneously, sequentially, or separately with a Pembrolizumab; and
wherein the cancer is selected an advanced or metastatic solid tumor.

51. (canceled)

52. The method according to claim 50, wherein the multispecific antibody comprises SEQ ID NO. 49 and SEQ ID NO. 67.

53-60. (canceled)

61. The method according to claim 50, wherein the antibody comprises a common light chain variable domain having an amino acid sequence as set forth in SEQ ID NO: 110, or a common light chain having an amino acid sequence as set forth in SEQ ID NO: 109.

62. The method according to claim 50, wherein the antibody comprises a heavy chain constant domain 1 (CHI) having an amino acid sequence as set forth in SEQ ID NO: 112 and having 0, 1, 2, 3, 4, 5, 6, 7, 8, 9 or 10 amino acid insertions, deletions, substitutions, or a combination thereof; a heavy chain constant domain 2 (CH2) having an amino acid sequence as set forth in SEQ ID NO: 114 and having 0, 1, 2, 3, 4, 5, 6, 7, 8, 9 or 10 amino acid insertions, deletions, substitutions, or a combination thereof; a heavy chain constant domain 3 (CH3) having an amino acid sequence as set forth in SEQ ID NO: 115 and having 0, 1, 2, 3, 4, 5, 6, 7, 8, 9 or 10 amino acid insertions, deletions, substitutions, or a combination thereof; and a heavy chain constant domain 3 (CH3) having an amino acid sequence as set forth in SEQ ID NO: 116 and having 0, 1, 2, 3, 4, 5, 6, 7, 8, 9 or 10 amino acid insertions, deletions, substitutions, or a combination thereof.

63. A pharmaceutical combination comprising a multispecific antibody that comprises an antigen binding site that binds an extracellular part of CD137, comprising a heavy chain variable domain comprising CDRs 1, 2, and 3 of SEQ ID NO: 49 and a light chain variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121); and an antigen binding site that binds an extracellular part of PD-L1 comprising a heavy chain variable domain comprising CDRs 1, 2, and 3 of SEQ ID NO: 67 and a light chain variable domain comprising an LCDR1 comprising the amino acid sequence QSISSY (SEQ ID NO: 120), an LCDR2 comprising the amino acid sequence AAS, and an LCDR3 comprising the amino acid sequence QQSYSTPPT (SEQ ID NO: 121), and instructions for use of the multispecific antibody in combination with Pembrolizumab.

64. The pharmaceutical combination according to claim 63, further comprising instructions for use.

65. The pharmaceutical combination of claim 63, wherein the instructions for use include instructions for intravenous administration and/or dosing amounts.


SEQUENCE LISTING (121 sequences)

SEQ ID NO: 1 - protein, 121 aa, synthetic construct
  source 1..121: note = Heavy chain variable region
QVQLVQSGSELKKPGASVKVSCKASGYTFTNFAMNWVRRAPGQGLEWMGWINTNTGNPTYAQGFTGRFVFSLDTSVNTAYLQISSLKAEDTAVYYCARDWGVIGGHYMDVWGKGTTVTVSS

SEQ ID NO: 2 - protein, 5 aa, synthetic construct
  source 1..5: note = HCDR1 according to Kabat from SEQ ID NO: 1
NFAMN

SEQ ID NO: 3 - protein, 17 aa, synthetic construct
  source 1..17: note = HCDR2 according to Kabat from SEQ ID NO: 1
WINTNTGNPTYAQGFTG

SEQ ID NO: 4 - protein, 12 aa, synthetic construct
  source 1..12: note = HCDR3 according to Kabat from SEQ ID NO: 1
DWGVIGGHYMDV

SEQ ID NO: 5 - protein, 121 aa, synthetic construct
  source 1..121: note = 5 Heavy chain variable region
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSDGYGPKAFDYWGQGTLVTVSS

SEQ ID NO: 6 - protein, 5 aa, synthetic construct
  source 1..5: note = HCDR1 according to Kabat
SYGIS

SEQ ID NO: 7 - protein, 17 aa, synthetic construct
  source 1..17: note = HCDR2 according to Kabat
WISAYNGNTNYAQKLQG

SEQ ID NO: 8 - protein, 12 aa, synthetic construct
  source 1..12: note = HCDR3 according to Kabat
DSDGYGPKAFDY

SEQ ID NO: 9 - protein, 123 aa, synthetic construct
  source 1..123: note = Heavy chain variable region
EVQLVQSGAEVKKPGESLKISCKGSGYSFTSYWIGWVRQMPGKGLEWMGIIYPDDSDTRYSPSFQGQVTISADKSSSTAYLQWSSLKASDTAMYYCASFYTGIVGATGAFDVWGQGTTVTVSS

SEQ ID NO: 10 - protein, 5 aa, synthetic construct
  source 1..5: note = HCDR1 according to Kabat
SYWIG

SEQ ID NO: 11 - protein, 17 aa, synthetic construct
  source 1..17: note = HCDR2 according to Kabat
IIYPDDSDTRYSPSFQG

SEQ ID NO: 12 - protein, 14 aa, synthetic construct
  source 1..14: note = HCDR3 according to Kabat
FYTGIVGATGAFDV

SEQ ID NO: 13 - protein, 128 aa, synthetic construct
  source 1..128: note = Heavy chain variable region
QVQLVQSGAEVKKPGSSVKVSCKASGGTFSSDAISWVRQAPGQGLEWMGGMIPILGTANYAQKFQGRVTITADRSTSTAYMELSSLRSEDTAVYYCVRGATYYYGSGTYYSINWFDPWGQGTLVTVSS

SEQ ID NO: 14 - protein, 5 aa, synthetic construct
  source 1..5: note = HCDR1 according to Kabat
SDAIS

SEQ ID NO: 15 - protein, 17 aa, synthetic construct
  source 1..17: note = HCDR2 according to Kabat
GMIPILGTANYAQKFQG

SEQ ID NO: 16 - protein, 19 aa, synthetic construct
  source 1..19: note = HCDR3 according to Kabat
GATYYYGSGTYYSINWFDP

SEQ ID NO: 17 - protein, 122 aa, synthetic construct
  source 1..122: note = Heavy chain variable region
QVQLVQSGSELKKPGASVKVSCRASGYTFTNFAMTWVRQAPGQGPEYMGWINTNTGNPTYAQGFTGRFVFSLDTSVNTAYLQISSLKAEDTAVYYCARDWASVMVRGDLDYWGQGTLVTVSS

SEQ ID NO: 18 - protein, 5 aa, synthetic construct
  source 1..5: note = HCDR1 according to Kabat
NFAMT

SEQ ID NO: 19 - protein, 13 aa, synthetic construct
  source 1..13: note = HCDR3 according to Kabat
DWASVMVRGDLDY

SEQ ID NO: 20 - protein, 126 aa, synthetic construct
  source 1..126: note = Heavy chain variable region
QVQLVQSGAEVKKPGASVKVSCKVSGYTLSELSIHWVRQAPGKGVEWMGGFYPEDVEPIYARKFQGRVTMTEDTSTDTAYMELNSLRSEDTAVYYCAAEGFDNYGSGIRGNWFDPWGQGTLVTVSS

SEQ ID NO: 21 - protein, 5 aa, synthetic construct
  source 1..5: note = HCDR1 according to Kabat
ELSIH

SEQ ID NO: 22 - protein, 17 aa, synthetic construct
  source 1..17: note = HCDR2 according to Kabat
GFYPEDVEPIYARKFQG

SEQ ID NO: 23 - protein, 17 aa, synthetic construct
  source 1..17: note = HCDR3 according to Kabat
EGFDNYGSGIRGNWFDP

SEQ ID NO: 24 - protein, 122 aa, synthetic construct
  source 1..122: note = Heavy chain variable region
EVQLVQSGAEVKKPGASVKVSCKVSGYTLTELSMHWVRQSPGKGLEWMGSFYPEDGETIYAQKFQGRITMTEDTSADTAYMELSSLRSEDTAVYYCATEGVGVIRGNWFDPWGQGTLVTVSS

SEQ ID NO: 25 - protein, 5 aa, synthetic construct
  source 1..5: note = HCDR1 according to Kabat
ELSMH

SEQ ID NO: 26 - protein, 17 aa, synthetic construct
  source 1..17: note = HCDR2 according to Kabat
SFYPEDGETIYAQKFQG

SEQ ID NO: 27 - protein, 13 aa, synthetic construct
  source 1..13: note = HCDR3 according to Kabat
EGVGVIRGNWFDP

SEQ ID NO: 28 - protein, 123 aa, synthetic construct
  source 1..123: note = Heavy chain variable region
EVQLVQSGAEVKKPGESLKISCKGSGYSFTSYWIGWVRQMPGKGLEWMGIIFPDDSDTRYSPSFQGQVTISADKSISTAYLQWSSLKPSDTAMYYCVRLGGYSGYAEDFVDFWGQGTLVTVSS

SEQ ID NO: 29 - protein, 17 aa, synthetic construct
  source 1..17: note = HCDR2 according to Kabat
IIFPDDSDTRYSPSFQG

SEQ ID NO: 30 - protein, 14 aa, synthetic construct
  source 1..14: note = HCDR3 according to Kabat
LGGYSGYAEDFVDF

SEQ ID NO: 31 - protein, 124 aa, synthetic construct
  source 1..124: note = Heavy chain variable region
EVQLVQSGAEVKKPGASVKVSCKVSGYTLTKLSMHWVRQAPGKGLEWMGGFEPEDGETINAQKFQGRVTMTEDTSTDTAYMELSSLRSEDTAVYYCATDLRLGASYYYSYMDVWGRGTMVTVSS

SEQ ID NO: 32 - protein, 5 aa, synthetic construct
  source 1..5: note = HCDR1 according to Kabat
KLSMH

SEQ ID NO: 33 - protein, 17 aa, synthetic construct
  source 1..17: note = HCDR2 according to Kabat
GFEPEDGETINAQKFQG

SEQ ID NO: 34 - protein, 15 aa, synthetic construct
  source 1..15: note = HCDR3 according to Kabat
DLRLGASYYYSYMDV

SEQ ID NO: 35 - protein, 121 aa, synthetic construct
  source 1..121: note = Heavy chain variable region
QITLKESGPTLVKPTQTLTLSCTFSGFSLSTSGMSVGWIRQPPGKALEWLALIYWNDDKYFSPSLKSRLTITKDTSKNQVVLTLTNMDPVDTATYYCAHTLWGSDDVFDVWGQGTMVTVSS

SEQ ID NO: 36 - protein, 7 aa, synthetic construct
  source 1..7: note = HCDR1 according to Kabat
TSGMSVG

SEQ ID NO: 37 - protein, 16 aa, synthetic construct
  source 1..16: note = HCDR2 according to Kabat
LIYWNDDKYFSPSLKS

SEQ ID NO: 38 - protein, 11 aa, synthetic construct
  source 1..11: note = HCDR3 according to Kabat
TLWGSDDVFDV

SEQ ID NO: 39 - protein, 123 aa, synthetic construct
  source 1..123: note = Heavy chain variable region
EVQLVQSGAEVKKPGESLKISCKVSGYSFTNYWIGWVRQMPGKGLEWMGIIYPGDSDTRYSPSFQGQVTISADKSISTAYLQWHTLKASDTAMYYCARHQGYSFSGSHIDDYWGQGTLVTVSS

SEQ ID NO: 40 - protein, 5 aa, synthetic construct
  source 1..5: note = HCDR1 according to Kabat
NYWIG

SEQ ID NO: 41 - protein, 17 aa, synthetic construct
  source 1..17: note = HCDR2 according to Kabat
IIYPGDSDTRYSPSFQG

SEQ ID NO: 42 - protein, 14 aa, synthetic construct
  source 1..14: note = HCDR3 according to Kabat
HQGYSFSGSHIDDY

SEQ ID NO: 43 - protein, 123 aa, synthetic construct
  source 1..123: note = Heavy chain variable region
EVQLVQSGAEVRKPGESLKISCKGSGYSFTTYWIGWVRQMPGKGLEWMGIIYPGDSDTRYSPSFQGQVTISADKSISTVYLQWSSLKASDTAMYYCARHAGFIITSQNIDDYWGQGTLVTVSS

SEQ ID NO: 44 - protein, 5 aa, synthetic construct
  source 1..5: note = HCDR1 according to Kabat
TYWIG

SEQ ID NO: 45 - protein, 14 aa, synthetic construct
  source 1..14: note = HCDR3 according to Kabat
HAGFIITSQNIDDY

SEQ ID NO: 46 - protein, 121 aa, synthetic construct
  source 1..121: note = Heavy chain variable region
EVQLVQSGSELKKPGASVKVSCKASGYTFTNFAMNWVRQAPGQGLEWMGWINTNTGNPTYAQDFTGRFVFSLDTSGNTAYLQISSLKAEDTAVYYCARDWGLVAIGYFDYWGQGTLVTVSS

SEQ ID NO: 47 - protein, 17 aa, synthetic construct
  source 1..17: note = HCDR2 according to Kabat
WINTNTGNPTYAQDFTG

SEQ ID NO: 48 - protein, 12 aa, synthetic construct
  source 1..12: note = HCDR3 according to Kabat
DWGLVAIGYFDY

SEQ ID NO: 49 - protein, 124 aa, synthetic construct
  source 1..124: note = Heavy chain variable region
QITLKESGPTLVKPTQTLTLTCTFSGFSLSTTGVGVNWIRQPPGEALEWLALIYWNDDTYYSPSLKSRLTITKDTSKNQVVLTMTNMDPVDTATYYCAHEGIIGFLGGNWFDPWGQGTLVTVSS

SEQ ID NO: 50 - protein, 7 aa, synthetic construct
  source 1..7: note = HCDR1 according to Kabat
TTGVGVN

SEQ ID NO: 51 - protein, 16 aa, synthetic construct
  source 1..16: note = HCDR2 according to Kabat
LIYWNDDTYYSPSLKS

SEQ ID NO: 52 - protein, 14 aa, synthetic construct
  source 1..14: note = HCDR3 according to Kabat
EGIIGFLGGNWFDP

SEQ ID NO: 53 - protein, 125 aa, synthetic construct
  source 1..125: note = Heavy chain variable region
QVQLVQSGSELKKPGASVKVSCKASGYTFTSHAMNWVRQAPGQGLEWMGWINPNTGNPTYAQGFTGRFVFSLDTSVSTAYLQISSLKAEDTAVYYCARDRKYVTNWVFAEDFQHWGQGTLVTVSS

SEQ ID NO: 54 - protein, 5 aa, synthetic construct
  source 1..5: note = HCDR1 according to Kabat
SHAMN

SEQ ID NO: 55 - protein, 17 aa, synthetic construct
  source 1..17: note = HCDR2 according to Kabat
WINPNTGNPTYAQGFTG

SEQ ID NO: 56 - protein, 16 aa, synthetic construct
  source 1..16: note = HCDR3 according to Kabat
DRKYVTNWVFAEDFQH

SEQ ID NO: 57 - protein, 125 aa, synthetic construct
  source 1..125: note = Heavy chain variable region
QVQLVQSGSELKKPGASVKVSCKASGYTFTSHAMNWVRQAPGQGLEWMGWINPNTGNPTYAQGFTGRFVFSLDTSVSTAYLQISSLKAEDTAVYYCAIDRGYMSNWVFAEYFPHWGQGTLVTVSS

SEQ ID NO: 58 - protein, 16 aa, synthetic construct
  source 1..16: note = HCDR3 according to Kabat
DRGYMSNWVFAEYFPH

SEQ ID NO: 59 - protein, 125 aa, synthetic construct
  source 1..125: note = Heavy chain variable region
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCATDRGYISSWVFAEDFQHWGQGTLVTVSS

SEQ ID NO: 60 - protein, 5 aa, synthetic construct
  source 1..5: note = HCDR1 according to Kabat
SYAMN

SEQ ID NO: 61 - protein, 16 aa, synthetic construct
  source 1..16: note = HCDR3 according to Kabat
DRGYISSWVFAEDFQH

SEQ ID NO: 62 - protein, 125 aa, synthetic construct
  source 1..125: note = Heavy chain variable region
QVQLVQSGSELKKPGASVKVSCTASGYTFTSYAMNWVRQAPGQRLEWMACVNPNTGSPTYAQGSTGRFVVSLDTSVSTAYLQISSLKAEDTAVYYCARDRKYVTNWVFAEDFQHWGHGTLVTVSS

SEQ ID NO: 63 - protein, 17 aa, synthetic construct
  source 1..17: note = HCDR2 according to Kabat
CVNPNTGSPTYAQGSTG

SEQ ID NO: 64 - protein, 125 aa, synthetic construct
  source 1..125: note = Heavy chain variable region
QVQLVQSGSELKKPGASVKVSCKASGYTFTNYAMNWVRQAPGQGLEWMGWMNPNTGNPTYAQGSTGRFVVSLDTSVSTAYLQISSLKAEDTAVYYCARDRKYVTNWVFAEDFQHWGRGTLVTVSS

SEQ ID NO: 65 - protein, 5 aa, synthetic construct
  source 1..5: note = HCDR1 according to Kabat
NYAMN

SEQ ID NO: 66 - protein, 17 aa, synthetic construct
  source 1..17: note = HCDR2 according to Kabat
WMNPNTGNPTYAQGSTG

SEQ ID NO: 67 - protein, 125 aa, synthetic construct
  source 1..125: note = Heavy chain variable region
QVQLVQSGSELKKPGASVKVSCKASGYTFTNYAINWVRQAPGQGLEWMGWINPNTGNPTYAQGFTGRFVFSLDTSVSTAYLQISSLKAEDTAVYYCARDRKYVTNWVFAEDFQHWGRGTLVTVSS

SEQ ID NO: 68 - protein, 5 aa, synthetic construct
  source 1..5: note = HCDR1 according to Kabat
NYAIN

SEQ ID NO: 69 - protein, 124 aa, synthetic construct
  source 1..124: note = Heavy chain variable region
EVQLVQSGAEVKKPGSSVKVSCKASGDTFNTYSITWVRQAPGQGLEWMGSIVPIFGTINNAQKFQGRVTITADKSANTAYMELSSLRSEDTAVYYCARDNTMVRGVDYYYMDVWGKGTMVTVSS

SEQ ID NO: 70 - protein, 5 aa, synthetic construct
  source 1..5: note = HCDR1 according to Kabat
TYSIT

SEQ ID NO: 71 - protein, 17 aa, synthetic construct
  source 1..17: note = HCDR2 according to Kabat
SIVPIFGTINNAQKFQG

SEQ ID NO: 72 - protein, 15 aa, synthetic construct
  source 1..15: note = HCDR3 according to Kabat
DNTMVRGVDYYYMDV

SEQ ID NO: 73 - protein, 121 aa, synthetic construct
  source 1..121: note = Heavy chain variable region
EVQLVQSGAEVKKPGSSVKVSCKASGGIFSTYAISWVRQAPGQGLEWMGGIIPIFDTPNYAQKFQGRVTITADKSTSTAYMDLSSLRSEDTAVYYCAKNVRGYSAYDLDYWGQGTLVTVSS

SEQ ID NO: 74 - protein, 5 aa, synthetic construct
  source 1..5: note = HCDR1 according to Kabat
TYAIS

SEQ ID NO: 75 - protein, 17 aa, synthetic construct
  source 1..17: note = HCDR2 according to Kabat
GIIPIFDTPNYAQKFQG

SEQ ID NO: 76 - protein, 12 aa, synthetic construct
  source 1..12: note = HCDR3 according to Kabat
NVRGYSAYDLDY

SEQ ID NO: 77 - protein, 119 aa, synthetic construct
  source 1..119: note = Heavy chain variable region
EVQLVQSGAEVKNPGSSVKVSCKATGGTFNTYGTNWVRQAPGQGLEWMGGIIPIFGTANYAQKFQGRVTITADKSTTTAYMEVSSLRSEDTAVYYCARGGADMGTLDYWGQGTLVTVSS

SEQ ID NO: 78 - protein, 5 aa, synthetic construct
  source 1..5: note = HCDR1 according to Kabat
TYGTN

SEQ ID NO: 79 - protein, 17 aa, synthetic construct
  source 1..17: note = HCDR2 according to Kabat
GIIPIFGTANYAQKFQG

SEQ ID NO: 80 - protein, 10 aa, synthetic construct
  source 1..10: note = HCDR3 according to Kabat
GGADMGTLDY

SEQ ID NO: 81 - protein, 120 aa, synthetic construct
  source 1..120: note = Heavy chain variable region
EVQLVQSGAEVMRPGSSVKVSCKASGGIFNTYTIIWVRQAPGQGLEWMGGIIPIFDTPNFAQKFQGRLTITADKSTNTAYMELTSLRSEDTAVYYCAREGCNHGVCYPYWGQGTLVTVSS

SEQ ID NO: 82 - protein, 5 aa, synthetic construct
  source 1..5: note = HCDR1 according to Kabat
TYTII

SEQ ID NO: 83 - protein, 17 aa, synthetic construct
  source 1..17: note = HCDR2 according to Kabat
GIIPIFDTPNFAQKFQG

SEQ ID NO: 84 - protein, 11 aa, synthetic construct
  source 1..11: note = HCDR3 according to Kabat
EGCNHGVCYPY

SEQ ID NO: 85 - protein, 121 aa, synthetic construct
  source 1..121: note = Heavy chain variable region
QVQLVQSGAEVKKPGSSVKVSCKASGDTFRSYGITWVRQAPGQGLEWMGGIIPIFGTTNYAQKFQGRVTITADKSTSTVYMELSSLRSEDTAVYYCARRRGYSNPHWLDPWGQGTLVTVSS

SEQ ID NO: 86 - protein, 5 aa, synthetic construct
  source 1..5: note = HCDR1 according to Kabat
SYGIT

SEQ ID NO: 87 - protein, 17 aa, synthetic construct
  source 1..17: note = HCDR2 according to Kabat
GIIPIFGTTNYAQKFQG

SEQ ID NO: 88 - protein, 12 aa, synthetic construct
  source 1..12: note = HCDR3 according to Kabat
RRGYSNPHWLDP

SEQ ID NO: 89 - protein, 119 aa, synthetic construct
  source 1..119: note = Heavy chain variable region
QVQLVQSGAEVKKPGSSVKVSCKASGGTFSTYGILWVRQAPGQGLEWMGGIIPIFGTANYAQKFQGRVTITADISTSTAYMELSSLRSEDTAVYYCARGGGNYYEFVYWGQGTLVTVSS

SEQ ID NO: 90 - protein, 5 aa, synthetic construct
  source 1..5: note = HCDR1 according to Kabat
TYGIL

SEQ ID NO: 91 - protein, 10 aa, synthetic construct
  source 1..10: note = HCDR3 according to Kabat
GGGNYYEFVY

SEQ ID NO: 92 - protein, 121 aa, synthetic construct
  source 1..121: note = Heavy chain variable region
EVQLVQSGAEVKKPGSSVRVSCKASGGTFNTYAINWVRQAPGQGLEWVGRIIPIFDTANYAQKFQGRVTISADKSTTTAYMELSSLRSEDTAVFYCAKDETGYSSSNFQHWGQGTLVTVSS

SEQ ID NO: 93 - protein, 5 aa, synthetic construct
  source 1..5: note = HCDR1 according to Kabat
TYAIN

SEQ ID NO: 94 - protein, 17 aa, synthetic construct
  source 1..17: note = HCDR2 according to Kabat
RIIPIFDTANYAQKFQG

SEQ ID NO: 95 - protein, 12 aa, synthetic construct
  source 1..12: note = HCDR3 according to Kabat
DETGYSSSNFQH

SEQ ID NO: 96 - protein, 125 aa, synthetic construct
  source 1..125: note = Heavy chain variable region
QVQLVQSGSELKKPGASVKVSCKASGYTFTNYAINWVRQAPGQGLEWMGWINPNTGNPTYAQGFTGRFVFSLDTSVSTAYLQISSLKAEDTAVYYCARDRKYVTNWVFAEDFQHWGQGTLVTVSS

SEQ ID NO: 97 - protein, 122 aa, synthetic construct
  source 1..122: note = Heavy chain variable region
QVQLVQSGAEVKRPGSSVKVSCKASGGTFNTYSITWVRQAPGQGLEWMGGIIPVFGTSKYAQKFQDRVTITADKSTNTAYMELSSLRSEDTAVYYCARDPSFSSSSGWFDPWGQGTLVTVSS

SEQ ID NO: 98 - protein, 17 aa, synthetic construct
  source 1..17: note = HCDR2 according to Kabat
GIIPVFGTSKYAQKFQD

SEQ ID NO: 99 - protein, 13 aa, synthetic construct
  source 1..13: note = HCDR3 according to Kabat
DPSFSSSSGWFDP

SEQ ID NO: 100 - protein, 121 aa, synthetic construct
  source 1..121: note = Heavy chain variable region
QVQLVQSGAEVKKPGSSVKVSCKASGGTFNTYAINWVRQAPGQGLEWMGGIIPIFDTANYAQRFQGRVTITADKSTSTAYMELSSLRSEDTAVYFCAKDQTGYSSTLFDYWGQGTLVTVSS

SEQ ID NO: 101 - protein, 17 aa, synthetic construct
  source 1..17: note = HCDR2 according to Kabat
GIIPIFDTANYAQRFQG

SEQ ID NO: 102 - protein, 12 aa, synthetic construct
  source 1..12: note = HCDR3 according to Kabat
DQTGYSSTLFDY

SEQ ID NO: 103 - protein, 125 aa, synthetic construct; identical to SEQ ID NO: 57
  source 1..125: note = Heavy chain variable region

SEQ ID NO: 104 - protein, 121 aa, synthetic construct
  source 1..121: note = Heavy chain variable region
EVQLVQSGAEVKKPGSSVKVSCKASGGTFSTYAISWVRQAPGQGLEWMGWIIPIFDTGNYAQKIQGRVTITADKSTSTAYMELTSLRSEDTAVYYCARHDYTNTVDAFDIWGQGTMVTVSS

SEQ ID NO: 105 - protein, 17 aa, synthetic construct
  source 1..17: note = HCDR2 according to Kabat
WIIPIFDTGNYAQKIQG

SEQ ID NO: 106 - protein, 12 aa, synthetic construct
  source 1..12: note = HCDR3 according to Kabat
HDYTNTVDAFDI

SEQ ID NO: 107 - protein, 121 aa, synthetic construct
  source 1..121: note = Heavy chain variable region
QVQLVQSGAEVKKPGSSVKVSCKASGDTFRSYGITWVRQAPGQGLEWMGGIIPVFGTTNYAQKFQGRVTITADKSTSTVFMELNSLRSEDTAVYYCARRRGYSNPHWLDPWGQGTLVTVSS

SEQ ID NO: 108 - protein, 17 aa, synthetic construct
  source 1..17: note = HCDR2 according to Kabat
GIIPVFGTTNYAQKFQG

SEQ ID NO: 109 - protein, 214 aa, synthetic construct
  source 1..214: note = Amino acid sequence of human common light chain IGKV1-39/jk1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 107 aa, synthetic construct
  source 1..107: note = Amino acid sequence of common light chain variable domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 111 - protein, 107 aa, synthetic construct [sequence omitted]

SEQ ID NO: 112 - protein, 98 aa, synthetic construct
  source 1..98: note = Amino acid sequence of CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 113 - protein, 15 aa, synthetic construct
  source 1..15: note = Amino acid sequence of the hinge
EPKSCDKTHTCPPCP

SEQ ID NO: 114 - protein, 110 aa, synthetic construct
  source 1..110: note = Amino acid sequence of CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 115 - protein, 107 aa, synthetic construct
  source 1..107: note = Amino acid sequence of CH3 with KK mutations
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 107 aa, synthetic construct
  source 1..107: note = Amino acid sequence of CH3 with DE mutations
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 255 aa, synthetic construct
  source 1..255: note = Amino acid sequence of CD137 according to HGNC: 11924
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 118 - protein, 447 aa, synthetic construct
  source 1..447: note = Amino acid sequence of Pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 218 aa, synthetic construct
  source 1..218: note = Amino acid sequence of Pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 6 aa, synthetic construct
  source 1..6: note = LCDR1
QSISSY

SEQ ID NO: 121 - protein, 9 aa, synthetic construct
  source 1..9: note = LCDR3
QQSYSTPPT